(19) United States
(12) Patent Application Publication
(10) Pub. No.: US 2012/0121633 A1
(43) Pub. Date: May 17, 2012

(54) HIV CD4 BINDING SITE BASED COVALENT IMMUNOGEN COMPOSITIONS

(76) Inventors: Sudhir Paul, Missouri City, TX (US); Yasuhiro Nishiyama, Houston, TX (US); Stephanie Planoue, Houston, TX (US); Richard J. Massey, Rockville, MD (US)

(21) Appl. No.: 13/184,530

(22) Filed: Jul. 16, 2011

Related U.S. Application Data
(60) Provisional application No. 61/365,139, filed on Jul. 16, 2010.

(51) Int. Cl. A61K 39/21; C07K 14/00; C07K 1/107; A61P 31/18; C12Q 1/70; C07K 7/08; C07K 16/10

(52) U.S. Cl. 424/188.1; 530/327; 530/326; 530/324; 530/389.4; 435/5

(57) ABSTRACT

Provided are immunogenic compositions based on the highly conserved, core CD4 binding site of the gp120 protein of the human immunodeficiency virus. One embodiment includes an antigenic conjugate of an electophilic derivative of HIV gp120 peptide 416-433, designated E-416-433, covalently linked to an immunogenic carrier protein. The compositions are effective in stimulating the production of HIV neutralizing antibodies in mammals. Provided also are related methods of immunization, methods of antibody production and antibodies obtained using the methods of the invention.

### CROSS-REFERENCE TO RELATED APPLICATIONS

This application claims the benefit of U.S. provisional patent application Ser. No. 61/365,139 filed Jul. 16, 2010, which is hereby incorporated by reference in its entirety.

### STATEMENT REGARDING FEDERALLY FUNDED RESEARCH

Pursuant to 35 U.S.C. §202(c) it is acknowledged that the U.S. Government has certain rights in the invention described herein, which was made in part with funds from the National Institutes of Health grants R01 AI067020; R01 AI058865.

### FIELD OF THE INVENTION

The invention relates to the fields of vaccination and immunotherapy with particular respect to the human immunodeficiency virus.

### BACKGROUND

Conventional Human Immunodeficiency Virus (HIV) vaccine approaches have proved ineffective because the immunodominant viral epitopes are mutable and the conserved epitopes necessary for infection are not sufficiently immunogenic. The CD4 binding site (CD4BS) of the HIV envelope protein of gp120 is a conserved sequence that is essential for viral entry into host cells. However, the development of an effective vaccine against this conserved epitope has been hindered for the following reasons.

The CD4BS core overlaps the B cell superantigen (SAg) site of gp120 recognized noncovalently by the framework regions (FRs) of antibodies (Abs) produced without prior HIV exposure, which results in super antigen suppression of an anti-CD4BS immune response due to defective IgM→IgG class switching and premature apoptosis rather than the desired B cell response. Study of HIV-infected humans and mice immunized with purified gp120 revealed down-regulated adaptive synthesis of Abs to the 421-433 CD4BS region characterized by deficient IgM→IgG class-switching. Nevertheless, long-term survivors of HIV infection do produce Abs to the 421-433 CD4BS region that neutralize genetically diverse HIV strains. While the development of such antibodies in these patients is very slow, continuing over decades during the course of infection, it shows that their endogenous production is not biologically impossible. The lack of proper conformational mimicry and stability by candidate CD4BS immunogens is believed to be another barrier to vaccines employing this epitope.

What is needed and provided herein are new immunogens and immunogenic compositions based on the core CD4BS sequence of gp120 that are effective in rapidly producing neutralizing Abs against HIV in mammals.

### SUMMARY OF THE INVENTION

One embodiment of the invention provides a synthetic immunogen with a conformation similar to the conformation of the CD4 binding site of gp120 on the surface of HIV that induces neutralizing antibodies to genetically diverse Group M HIV-1 strains, having the formula

L-E

wherein L is a peptide sequence that comprises fourteen or more amino acids selected from the region of gp120 containing the consensus amino acids 406-459 numbered according to the HXB2 numbering system, or a mimotope thereof, and E is an electrophilic group covalently linked to an amino acid side chain of L, having the formula

Y-Y′-Y″

wherein

Y is an electrophilic group,

Y′ is a charged or neutral group, or is absent, and

Y″ is a linker, covalent bond or atom.

The synthetic immunogen is effective to induce the synthesis of HIV neutralizing antibodies to the 421-433 CD4 binding site sequence by recognition of the framework regions of B cell receptors. In one variation L comprises peptide sequence 416-433 of gp120. In another variation L comprises peptide sequence 414-439.

In a related embodiment of the invention, the synthetic immunogen has the formula:

Cys-Leu-Pro-Ser-Arg-Ile-Lys(X)-Gln-Ile-Ile-Asn-Met-Trp-Gln-Glu-Val-Gly-Lys(X)-Ala, or

Ile-Thr-Cys-Leu-Pro-Ser-Arg-Ile-Lys(X)-Gln-Ile-Ile-Asn-Met-Trp-Gln-Glu-Val-Gly-Lys(X)-Ala-Met-Tyr-Ala-Pro-Pro-Ile,

wherein X is an electrophilic group of formula:

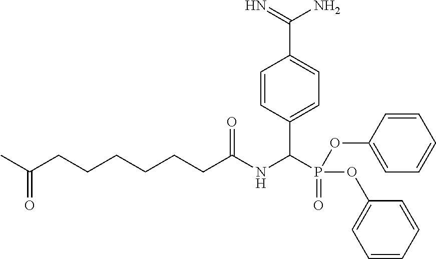

and Lys(X) indicates X is covalently linked to a side chain of the lysine residue.

A further embodiment of the invention provides a synthetic immunogen with a conformation similar to the conformation of the CD4 binding site of gp120 on the surface of HIV that induces neutralizing antibodies to genetically diverse Group M HIV-1 strains, having the formula

L-E

wherein L is a peptide sequence that comprises fourteen or more amino acids selected from the region of gp120 containing amino acids 406-459 with one or more amino acid sequence differences compared to the consensus sequence of amino acids 406-459 of Group M HIV-1 gp120, and E is an electrophilic group covalently linked to an amino acid side chain of L, having the formula

Y-Y′-Y″

wherein

Y is an electrophilic group,

Y′ is a charged or neutral group, or is absent, and

Y″ is a linker, covalent bond or atom.

The invention also provides immunogenic composition that include an immuogen of the invention and an adjuvant.

In another variation of the above embodiments, L is a peptide sequence that comprises fourteen or more amino acids selected from the region of gp120 containing amino acids 406-459 or a mimotope thereof and a second epitope of gp120 or a mimotope thereof.

The synthetic immunogens of the invention may further include one or more cross-links between amino acids that rigidify the conformation.

The immunogens of the invention may be conjugated to one or more carrier molecules. The carrier molecule may, for example, be the carrier molecule is selected from the group consisting of keyhole limpet hemocyanin, tetanus toxoid, and CD40 ligand. The carrier molecule promotes folding of the synthetic immunogen into a conformation similar to the conformation of the CD4 binding site of gp120 on the surface of HIV.

Another embodiment of the invention provides a method for producing HIV neutralizing antibodies to genetically diverse Group M HIV-1 strains within an organism capable of producing antibodies, such as a mammal, for example, a mouse, rabbit, monkey or human including the step of: administering an immunogen of the invention to the organism in an amount effective to cause production of neutralizing antibodies against the CD4-binding site of HIV gp120. The administering step may further include administering an adjuvant to the mammal.

A further embodiment of the invention provides a method for producing HIV neutralizing antibodies to genetically diverse Group M HIV-1 strains within an organism capable of producing antibodies, such as a mammal, for example, a mouse, rabbit, monkey or human including the step of: administering an immunogen according to the invention to the organism in an amount effective to cause production of neutralizing antibodies against the CD4-binding site of HIV gp120. The administering step may further include administering an adjuvant to the organism.

A further embodiment of the invention provides a method for the preparation of an immunogen with a conformation similar to the CD4 binding site of gp120 on the surface of HIV that induces neutralizing antibodies to genetically diverse Group M HIV-1 strains, said immunogen having the formula

L-E

wherein L is gp120 and E is an electrophilic group conjugated to a side chain functional group of L having the formula

Y-Y′-Y″

wherein

Y is an electrophilic group,

Y′ is a charged or neutral group, or is absent, and

Y″ is a linker, covalent bond or atom, comprising the steps of:

(a) conjugating varying numbers of Y-Y′Y″ groups per molecule of gp120;

(b) incubating the resultant L-E preparation for a length of time sufficient to enable intermolecular covalent bonding between the L-E molecules

(c) fractionating the L-E preparation into multiple fractions containing individual subpopulations of L-E molecules characterized by their size, charge, hydrophobicity or conformation;

(d) assaying the several variant L-E fractions from step (c) to determine their CD4 binding activity or their antibody binding activity; and

(e) identifying those variant L-E fractions from step (c) that induce the greatest synthesis of HIV neutralizing antibodies in an organism.

Another embodiment of the invention provides a method for the preparation of an immunogen that induces neutralizing antibodies to genetically diverse Group M HIV-1 strains, said synthetic immunogen composed of intact HIV-1 particles having on their surface molecules with the formula

L-E

wherein L is gp120 and E is an electrophilic group conjugated to a side chain functional group of L having the formula

Y-Y′-Y″

wherein

Y is an electrophilic group,

Y′ is a charged or neutral group, or is absent, and

Y″ is a linker, covalent bond or atom, comprising the steps of:

(a) conjugating varying numbers of Y-Y′Y″ groups per gp120 molecule expressed on the surface of intact HIV-1;

(b) incubating the resultant HIV-1 particles for a length of time sufficient to enable intermolecular covalent bonding between the surface L-E molecules

(c) fractionating the HIV-1 particles with surface L-E molecules into multiple fractions containing individual subpopulations of HIV-1 particles characterized by their size, charge, hydrophobicity or conformation;

(d) assaying the several variant HIV-1 fractions from step (c) to determine their CD4 binding activity or their antibody binding activity; and

(e) identifying those variant HIV-1 fractions from step (c) to induce the greatest synthesis of HIV neutralizing antibodies in an organism.

Another embodiment provides an isolated polypeptide, such as but not limited to an antibody, comprising the framework regions (non-underlined portions) of at least one of the following antibody V_{L} and V_{H} amino acid sequences:

IgM clone G12
(a) VL chain:
(SEQ ID NO: __)
ENVLTQSPAIMSASPGEKVTMTCSASSSVSYMHWYQQKSSTSPKLWIYDT
SKLASGVPGRFSGSGSGNSYSLTISSMEAEDVATYYCFQGSGYPYTFGGG
TKLEIK;
(b) VH chain:
(SEQ ID NO: __)
QVQLQQPGSVLVRPGASVKLSCKASGYTFTSSWMHWAKQRPGQGLEWIGE
IHPNSGNTNYNEKFKGKATLTVGTSSSTAYVDLSSLTSEDSAVYYCARPG
IGESQSFPNVFPAAAEXLKGEFCRYPSHWRPLEHAS;
IgM clone C11:
(c) VL chain:
(SEQ ID NO: __)
DIQMTQSPATLSVTPGDSVSLSCRASQSISNNLHWYQQKSHESPRLLIKY
ASQSISGIPSRFSGSGSGTDFTLSINSVETEDFGMYFCQQSNSWPLTFGA
GTKLELK;
(d) VH chain:
(SEQ ID NO: __)
VQVQLKQSGPGLVQPSQSLSITCTVSGFSLTSYGVHWVRQSPGKGLEWLG
VIWSGGSTDYNAAFISRLSISKDNSKSQVFFKMNSLQANDTAIYYCARTG
FAYWGRGTLVTVS;
IgM clone H10:
(e) VL chain:
(SEQ ID NO: __)
QIVLTQSPAIMSASLGERVTMTCTASSSVSSSYLHWYQQKPGSSPKLWIY
STSNLASGVPARFSGSGSGTSYSLTISSMEAEDAATYYCHQYHRSPRTFG
GGTKLEIK;
(f) VH chain:
(SEQ ID NO: __)
EVKLVESGGGLVQPGGSLRLSCATSGFTFTDYYMSWVRQPPGKALERLGF
IRNKANGYTTEYSASVKGRFTISRDNSQSILYLQMNTLRAEDSATYYCAR
DNQSFYYAMDYWGQGTSVTVSS;
IgM clone 1F4:
(g) VL chain:
(SEQ ID NO: __)
VLMTQTPLSLPVSLGDQASISCRSSQSLVHSNGNTYLHWYLQKPGQSPKL
LIYKVSNRFSGVPDRFSGSGSGTDFTLKISRVEAEDLGVYFCSQSTHVPY
TFGGGTKLEIK;
(h) VH chain:
(SEQ ID NO: __)
EVKLQESGPSLVQPSQSLSITCTVSGFSLTSYGVHWVRQSPGKGLEWLGV
IWRGGSTDYNAAFMSRLSITKDNSKSQVFFKMNSLQADDTAIYYCAKRYG
NYGGGAMDYWGQGTSVTVSS;
IgM clone 2C11:
(i) VL chain:
(SEQ ID NO: __)
QIVLTQSPAIMSASPGEKVTITCSASSSVSYMHWFQQKPGTSPKLWIYST
SNLASGVPARFSGSGSGTSYSLTISRMEAEDAATYYCQQRSSYPYTFGGG
TKLEIK;
(j) VH chain:
(SEQ ID NO: __)
EVQLVESGGGLVQPKGSLKLSCAASGFTFNTYAMHWVCQAPGKGLECVAR
IRSKSNNYATYYADSVKDRFTISRDDSQSMLYLQMNNLKTEDTAMYYCVR
ERAGYFDVWGAGTTVTVSS;
IgM Clone 2G2:
(k) VL chain:
(SEQ ID NO: __)
DIVITQSPSYLAASPGETITINCRASKSISKYLAWYQEKPGKTNKLLIYS
GSTLQSGIPSRFSGSGSGTDFTLTISSLEPEDFAMYYCQQHNEYPYTFGG
GTKLEIK;
(l) VH chain:
(SEQ ID NO: __)
EVQLQQSGPELVKTGASVKISCKASGYSFTGYYMHWVKQSHGKSLEWIGY
ISCYNGATSYNQKFKGKATFTVDTSSSTAYMQFNSLTSEDSAVYYCARGG
TTVVATGKYAMDYWGQGTSVTVSS;
IgM clone 2G9:
(m) VL chain:
(SEQ ID NO: __)
DIQMTQTTSSLSASLGDRVTISCRASQDISNYLNWYQQKPDGTVKLLIYY
TSRLHSGVPSRFSGSGSGTDYSLTISNLEQEDIATYFCQQGNTLPTFGGG
TKLEIKRA;
(n) VH chain:
(SEQ ID NO: __)
QVQLKQSGPGLVQPSQSLSITCTVSGFSLTSYGVHWVRQSPGKGLEWLGV
IWSGGSTDYNAAFISRLSISKDNSKSQVFFKMNSLQANDTAIYYCARNKD
YGSSYDYYAMDYWGQGTSVTVSS;
IgG clone 9F3:
(o) VL chain:
(SEQ ID NO: __)
DIVMSQSPSSLAVSAGEKVTMRCKSSQSLLNSRTRKNYLAWYQQKPGQSP
KLLIYWASTRESGVPDRFTGSGSGTDFTLTISSVQAEDLAVYYCKQFYNL
WTFGGGTKLEIK;
(p) VH chain:
(SEQ ID NO: __)
QVQLQQSGAELVRPGASVKLSCKALGYTFTDYEMHWVKQTPVHGLEWIGG
IYPGSGGTAYNQKFKGKATLTADKSSSTAYMELSSLTSEDSAVYYCTKFR
FSSFAMDYWGQGTSVTVSS;
IgG clone 4B2:
(q) VL chain:
(SEQ ID NO: __)
DIVMSQSPSSLAVSAGEKVTMSCKSSQSLLNSRTRKNYLAWYQQKPGQSP
KLLIYWASTRESGVPDRFTGSGSGTDFTLTINSVQAEDLAVYYCKQSYNL
WTFGGGTKLEIK;
and
(r) VH chain:
(SEQ ID NO: __)
QVQLQQSGAELVRPGASVKLSCMALGYTFTDYEIHWVKQTPVHGLEWIGG
FHPGSGGGAYSQKFKGKATLIADKSSSIAYMEVISLTSEDSAVYYCTRFR
YSSFAMVYWGQGTSVTVSS,

wherein underlined sequences are CDRs and non-underlined sequences are antibody framework regions. A related embodiment provides an isolated polypeptide such as but not limited to an antibody that includes at least one of the aforementioned V_{H} or V_{L} sequences. The aforementioned polypeptides may be administered to a human for the prophylaxis or treatment of HIV infection.

Another embodiment of the invention provides an isolated preparation of polyclonal antibodies obtained following immunization of an organism capable of producing antibodies, such as a mammal, with an immunogen or immunogenic composition.

A further embodiment of the invention provides a method of prophylaxis or treatment against HIV infection that includes the step of administering an immunogen or immunogenic composition as described herein to human being in an amount effect to induce the production of HIV neutralizing antibodies. Use of an immunogen or immunogenic composition as described herein for the prophylaxis or treatment of HIV infection in a human is similarly provided.

Another embodiment of the invention provides a method of prophylaxis or treatment of HIV infection that includes the step of administering to a human monoclonal or polyclonal antibodies as described herein or polypeptides. Use of such antibodies for the prophylaxis or treatment of HIV infection in a human is similarly provides.

The invention also provides the use of any of the immunogens, antibody sequences, and antibodies described herein for the preparation of a medicament for the prophylaxis or treatment of HIV infection in a human.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1. Exposure of the 421-433 CD4BS sequence on gp120. Shown is a surface representation of gp120 crystal structure (PDB 2B4C; after stripping away bound soluble CD4 and Fab X5), in which the 421-433 CD4BS surface is highlighted in green. Of the total 421-433 surface area, ˜85% is exposed (measured in silico as the water accessible surface area). The neighboring β15/β2 strands are shown in purple.

FIG. 2. Role of immunogen and BCR conformational flexibility in successful induction of neutralizing Abs. Top, A flexible immunogen can take a shape complementary to the shape of the BCR combining site by the induced-fit mechanism, thereby losing its CD4BS-mimicking conformation and resulting in induction of non-neutralizing Abs. Bottom, A rigid, improved E-immunogen mimicking the CD4BS conformation will induce the correct BCR conformation that can be affinity-matured by immunogen-driven selection, resulting in adaptive synthesis of affinity-matured neutralizing Abs.

FIG. 3: Schematic representation of probes and control polypeptides used in this invention. Single letter code is used for amino acid designation. BtLC is a biotin linker that is attached at the N terminus of the polypeptide and can be employed to detect binding to Ab or CD4 using streptavidin/biotin detection on denaturing electrophoresis. A cysteine (C) is introduced at the N terminus of the polypeptide to permit the conjugation to proteins such as keyhole limpet hemocyanin (KLH) or bovine serum albumin (BSA). Details on preparation of the above reagents can be found in ref 1-4.

FIG. 4: Structure of improved E-immunogens. A, KLH-E-416-433. E signifies phosphonate groups placed at Lys side chains. Single letter code is used to designate amino acids. A cysteine (C) is introduced at the N terminus of the polypeptide to permit the conjugation to keyhole limpet hemocyanin (KLH). B, E-gp120. E-gp120 contains E groups at Lys side chains. Inset shows silver-stained covalent oligomers of unfractionated E-gp120 (lane 1), the trimer-enriched E-gp120 fraction from gel filtration (lane 2), and control gp120 devoid of phosphonate groups (lane 3).

FIG. 5: Improved 421-433 CD4BS conformation expressed by E-gp120. Binding of immobilized gp120 and E-gp120 by anti-421-433 CD4BS sequence MAb JL427 determined by ELISA. E-gp120 binding measured in the absence or presence of excess E-hapten 1 (100 μM, see FIG. 3) to eliminate covalent binding effects.

FIG. 6. Split-site model explaining proteolytic Ab epitope specificity. Two different Ab subsites are responsible for the initial noncovalent antigen binding and the subsequent peptide bond hydrolysis process. In the initial immune complex (left), the antigen region not involved in noncovalent antibody binding enjoys conformational flexibility. Consequently, peptide bonds remote from the noncovalent binding site that are in register with the Ab nucleophilic subsite can be hydrolyzed (right). The E-immunogens presented in this invention contain phosphonate groups that form a covalent bond with the Ab nucleophile following the initial noncovalent binding. Triangle, nucleophile; Circle, neighboring general base that activates the nucleophile. See refs 5-8 for further details.

FIG. 7. Covalent vaccine principle. Traditional non-electrophilic immunogens induce a transient Ab response to the 421-433 CD4BS sequence limited mostly to the IgM compartment because of deficient class-switching (left panel). The electrophilic phosphonate of the example E-416-433 immunogen binds BCR nucleophiles (Nu) via the highly energetic covalent reaction, bypassing constraints on B cell differentiation (middle panel). This generates memory B cells and plasma cells producing neutralizing Abs. An optional epitope 2 that can be incorporated in the E-vaccine generates a positive signal by binding the CDRs, which counteracts negative B cell signaling due to 421-433 CD4BS sequence binding at the FRs (upper pink panel). Electrophile-driven clonal selection of the B cells results in adaptive strengthening of Ab nucleophilic reactivity, improving the innate catalytic activity of Abs (lower pink panel). Specificity is derived from noncovalent epitope-paratope binding. Covalent immune complex 1, resonance stablized complex formed prior to expulsion of C-terminal antigen fragment. Covalent immune complex 2, acyl-Ab complex. Ag′Lys-OH, N-terminal antigen fragment; NH2-Ag″, C-terminal antigen fragment.

FIG. 8. Preimmune Abs specific to 421-433 CD4BS epitope. Enriched HIV neutralizing activity of epitope-specific Ab fractions from preimmune mouse serum purified by affinity chromatography on agarose-conjugated E-416-433. Plotted are the Ab concentrations for individual fractions at which 50% inhibition of infection occurred (computed from Ab dose-response curves). HIV strain, 97ZA009. Host cells, PBMCs. Ab content determined as the sum of IgM, IgG and IgA measured by ELISA. ‘Starting serum’ refers to unfractionated serum (1 ml) used for affinity purification on agarose-conjugated E-416-433 (0.7 μmmol/mL gel). After removing unbound proteins at neutral pH, noncovalently bound Abs were eluted with 0.1 M glycine-HCl, pH 2.7, followed by recovery of the covalently bound Abs by treatment with 10 μM PAM. Inset, Specific covalent binding of Bt-E-421-433 (10 μM) to polyclonal IgM (150 nM) from a human without HIV infection in the absence (lane 1) and presence of competitor 421-436 peptide (500 μM, lane 2). Streptavidin-peroxidase stained reducing SDS-gels. Data from ref 9.

FIG. 9. gp120 recognition by antibody framework regions (FRs). A, Schematic structures of single chain Fv (scFv) clone JL427, scFv GL2 and their FR/CDR swap mutants. FR1, FR3 and CDR1 from the VH3-family scFV JL427 were inserted in place of the corresponding regions of the VH4-family scFv GL2 by mutagenesis. B, Improved E-gp120 recognition by scFv GL2 swap mutants containing FR1 and FR3 from scFv JL427. ELISA data showing scFv binding to immobilized E-gp120 (100 ng/well). E-gp120, gp120 containing electrophilic phosphonates at Lys residues (See FIG. 4). scFv clones purified by metal affinity chromatography. Detection with Ab to c-myc tag at the C terminus of the scFv.

FIG. 10. Soluble CD4 reactivity of electrophilic 421-433 CD4BS sequence mimetics. A, Specific binding of sCD4 to immobilized KLH-E-416-433 and the shorter KLH-E-421-433. KLH-E-416-433 and KLH-E-421-433, 0.3 μmol/well. sCD4 binding determined using rabbit anti-CD4 Ab in the presence of 100 μM E-hapten 1. KLH-E-VIP, an irrelevant electrophilic peptide described in ref 3. B, Competitive inhibition of sCD4 binding to immobilized KLH-E-416-433 by soluble Bt-E-416-433 and Bt-NE-416-433 epitope mimetics (see FIG. 3). Bt-NE-416-433, non-electrophilic biotinylated peptide without phosphonate groups. sCD4 binding was determined as in panel A.

FIG. 11. Deficient IgM→IgG class switching of Ab response to 421-433 CD4BS epitope in HIV-infected humans and gp120-immunized mice. A, BSA-E-416-433 binding by IgM or IgG purified from the sera of HIV infected patients 0.5-5 years post-seroconversion determined by ELISA (n=10). Abs, 30 μg/ml. Each closed symbol represents the indicated Ab fraction from one patient. Open circles, pooled Igs from 11 noninfected subjects. B, Full-length gp120 binding of the same human IgM and IgG samples described in panel A determined by ELISA. IgG 10 μg/ml; IgM 30 μg/ml. C, E-416-433/gp120 binding ratios for human IgM and IgG computed from Panels A and B. Mann-Whitney U-test, two tailed. D, Example BSA-E-416-433 binding curves of increasing BALB/c mouse serum IgM and IgG concentrations induced by immunization with full-length gp120 (4 intraperitoneal immunizations with 11 μg strain MN gp120 at 2 week intervals in RIBI adjuvant). IgM/IgG purified using anti-IgM/Protein G columns. E, BSA-E-416-433 binding by IgM or IgG from the sera of mice immunized with full-length gp120 as in Panel D. Each point represents data from pooled sera of 4 mice each. Data are from 3 independent mouse immunization experiments. F, Full-length gp120 binding of the same mouse IgM and IgG samples described in panel E determined by ELISA.

FIG. 12. Heterologous strain neutralization by IgA from patients with HIV infection. A, 416-433 and 301-324 epitope sequences from the autologous subtype B strain of IgA donor 2866 and heterologous subtype C strain ZA009 used for neutralization assays. Divergences between the sequences of heterologous ZA009 strain V1, V2, V3, V4 and V5 domains and the corresponding domains of the autologous virus from 3 patients with infection for 19-21 years (I₁₉₋₂₁ patients) were 35-87%. Corresponding divergence values for the 416-433 epitope were 6-17%. B, IgA neutralizing potency versus duration of infection. Each data point corresponds to IgA from the serum of a different HIV infected donor. Host cells, PBMCs; virus isolate, R5-dependent subtype C strain ZA009. IC50, 50% neutralization concentration. Dashed lines, 95% confidence limits. C, Enriched neutralizing activity of epitope-specific IgA fractions. Pooled IgA (I₁₉₋₂₁ donors 2857, 2866 and 2886) was fractionated on agarose-conjugated as in FIG. 8. Values are means±s.e.m. of 4 replicates. Host cells, PBMCs; virus, strain ZA009.

Table 1. 416-433 epitope recognition by neutralizing IgA from HIV infected patients and a neutralizing murine MAb raised by KLH-E-416-433 immunization. The table shows that the gp120 amino acids important for recognition of CD4 are also critical for recognition of the neutralizing Abs, supporting recognition of the 421-433 CD4BS sequence as the mechanism of neutralization. Sequence conservation determined from 1699 group M strains (http://www.hiv.lanl.gov/). Conservative mutations defined as in BLAST; I/L-P-C-R/K-I-K/R-Q-I/V-I/V-N/D-M/L-W-Q-E/D/Q-V/I-G-K/Q/R-A). Dotted boxes indicate residues important for sCD4 binding (dots) or Ab binding determined by crystallography or by mutagenesis (reduction of binding by >50%). Values for I₁₉₋₂₁ IgA and MAb IgM 2G9 correspond to the ratio (IC50_{wild type peptide}/IC50_{mutant peptide}) where IC50 is the concentration of soluble wildtype 416-433 peptide or its Ala mutant at each position displaying 50% competitive inhibition of Ab binding to immobilized KLH-E-416-433. Yellow boxes, non-contacting residues or <50% loss of binding activity upon mutagenesis. sCD4 data are from refs 10 and 11 and PDB structures 1GC1, 1G9M, 1G9N, 2QAD, 2B4C. MAb IgM 2G9 obtained by immunization with KLH-E-416-433.

FIG. 13. Neutralization of 18 genetically diverse CCRS-dependent primary HIV strains, SHIVSF162P3 and 3 HIV molecular clones by IgA purified from 3 survivors of prolonged HIV infection (19-21 years, 119-21 patients). Shown are scatter plots of neutralization potencies for individual strains (IC50) of the IgA preparations and the reference anti-CD4BS IgG b12 (see patient and strain details in ref2).

FIG. 14. Unimpaired IgM→IgG class-switching of Abs to the 421-433 CD4BS epitope induced by covalent immunization: BSA-E-416-433 binding by IgG and IgM from E-gp120 and E-HIV immunized mice. A, Example of BSA-E-416-433 binding by increasing concentrations of IgG and IgM from mice immunized with E-120. ELISA and immunization protocol as in FIG. 11D. B, IgM→IgG class-switch ratio (CS) for the Ab responses induced by gp120 and E-gp120. CS ratio defined as [(A490 E-416-433 binding per μg IgG/A490 E-416-433c binding per μg IgM)÷(A490 gp120 binding per μg IgG/A490 gp120 binding per μg IgM A490)]. Values are means±SEM from 3 independent mouse immunizations each conducted by identical protocols for the gp120 or E-gp120 immunogens. Binding of BSA-E-416-433 and gp120 determined by ELISA using increasing IgM and IgG concentrations. C, BSA-E-416-433 binding activity IgG and IgM from mice immunized with E-HIV (mean±SEM of data from 5 mice). Five intraperitoneal immunizations with 50 ng gp120 equivalents of E-HIV at weekly intervals in RIBI. E-HIV, psoralen-inactivated strain 111B virions labeled with phosphonate groups at surface Lys side chains as described for gp120 in ref 6 at a ratio of 106 mol phosphonate/mol gp120. gp120 content of E-HIV determined by ELISA. Virions obtained as the void volume fraction from a gel filtration column for labeling with the phosphonate.

FIG. 15. Structural properties of a MAb to the immunodominant 421-433 CD4BS sequence. MAbs were from E-gp120 immunized mice identified by irreversible binding to E-gp120. A, Model of anti-E-gp120 Fab YZ23 (2.5 Å resolution) showing the CDR-cavity and VH FR cavity fitted for clarity, respectively, with the red-meshed and white-meshed objects. VL domain, pink; VH domain, cyan; CDRL1 and L3, respectively, yellow and orange; CDRH1, H2 and H3, respectively, blue, light green and dark green. Inter-cavity distance and cavity surface areas are indicated. B, Reduced binding of immobilized BSA-E-416-433 by the FR1 VH Lys19:Ala mutant of single chain Fv YZ23. Mean values after subtraction of nonspecific binding determined using ELISA wells coated with BSA are shown. *, P<0.05; **, P<0.006. Data from ref 12. C, Immunodominance of the 421-433 CD4BS sequence expressed by E-gp120. Shown are values of the E-416-433 binding Ab subsets induced by immunization of mice with gp120 or E-120 determined by adsorbing serum IgG on immobilized agarose-E-416-433 and measuring binding to gp120 and E-gp120, respectively. % Abs directed to the 421-433 CD4BS sequence were computed as [100−(100×A490 adsorbed IgG/A490 starting IgG)] tested at equivalent adsorbed and starting IgG concentrations.

FIG. 16: Immunogen properties. A, gp120 and E-120 inhibition of HIV binding by MAbs to reference neutralizing epitopes (MAb clones b12 to the outer domain CD4BS epitope, MAb 447-52D to the V3 epitope and MAb 2G12 to a carbohydrate-dependent epitope). The MAbs were incubated for 20 hours with intact HIV virions (strain MN 1.6×103 TCID50/ml; MAb clone b12 45 μg/ml; clone 447-52D 0.8 μg/ml; clone 2G12 7.5 μg/ml) in the presence or absence of gp120, E-gp120, EGF and E-EGF (0.5 μM). MAb-HIV complexes were captured using immobilized Protein G and viral p24 was measured by ELISA as in ref 13. Plotted are values of residual HIV binding (%; mean±S.D. of 4-6 replicates) defined as 100×(A490 in the presence of competitor)/(A490 in the absence of competitor). All values are corrected for nonspecific binding observed in diluent instead of the MAbs. MAb binding (A490) in the absence of competitors were: clone b12, 0.176±0.018; clone 447-52D, 0.291±0.085; clone 2G12, 0.246±0.046). B, Binding of immobilized gp120 and E-gp120 to MAb b12 and MAb 268-DIV determined by ELISA (MAb clone b12, 10 μg/ml; clone 268-DIV, 2 μg/ml).

FIG. 17: Flowchart for IE-gp120 preparation.

FIG. 18. BSA-E-416-433 binding titers of polyclonal Abs from KLH-E-416-433 immunized mice and rabbits (ELISA). A, Binding activity of IgM, IgG and IgA from serum and IgA from cervico-vaginal lavage fluid (CVLF, Inset) induced in mice by intranasal immunizations (arrows) with KLH-E-416-433 using E. Coli heat labile enterotoxin mutant as adjuvant (LTm, ref 14; pool of 8 mice). B, Serum IgM, IgG and IgA titers in a rabbit immunized subcutaneously with KLH-E-416-433. Titer is defined as the dilution giving an A490 binding value of 0.2.

Table 2. Neutralization of genetically diverse HIV strains by polyclonal serum Abs (unfractionated serum) from a rabbit and mice immunized with KLH-E-416-433. Titer, dilution at which 50% neutralization occurred. Data are from plots of varying serum dilution versus neutralization fitted to the equation % neutralization=100/(1+10(logIC50−log [Ab])·Hillslope). PBMC host cells. The gp120 V domains of these strains are highly divergent (values shown are computed using strain 97ZA009 as reference). Sequence alignments were with ClustalW2 program. NA, not available; Underlined red, non-conservative replacements compared to the immunogen. Underlined green, conservative replacements; X, unidentified amino acid.

FIG. 19. Enriched neutralizing activity of epitope-specific Ab fractions of serum from KLH-E-416-433 immunized mice. Abs were fractionated by affinity chromatography on agarose-conjugated E-416-433. Plotted are the neutralization potencies (IC50) expressed per unit mass Ab content (sum of IgM, IgG and IgA measured by ELISA using class-specific Abs as in FIG. 8). IC50 computed from dose-response curves using the CCR5-dependent subtype C strain 97ZA009. Reduced IC50 shows increased potency. ‘Starting’ refers to unfractionated serum. Noncovalently and covalently bound fractions were recovered, respectively, by pH 2.7 elution and 2-PAM elution.

FIG. 20. Binding of intact, infectious subtype C 97ZA009 virions by polyclonal rabbit IgG induced by KLH-E-416-433 immunization. After incubation, HIV-IgG complexes were recovered on Protein G columns and p24 in eluates was measured as in ref 13. Plotted are the relative light units (RLU) captured by preimmune and immune IgG.

Table 3. Neutralization of murine anti-E-416-433 MAbs raised from KLH-E-416-433 immunization. PBMC host cells. Data are from plots of varying MAb concentration versus neutralization. IC50 is defined as the MAb concentration showing 50% neutralization. NA, not analyzed.

FIG. 21. Competitive inhibition of MAb 2G9 binding to immobilized BSA-E-416-433 by sCD4 and full-length gp120 (subtype B strain MN). IgM 2G9 binding to BSA-E-416-433 (70 ng/well) was tested in the absence or presence of increasing concentrations of Bt-E-416-433, sCD4, gp120, control Sh416-433 and control ovalbumin (OVA). Y-axis values are computed as follows 100x[A490 with competitor/A490 without competitor]. Methods as in ref 2.

FIG. 22. gp120 cleavage by anti-KLH-E-416-433 MAb H10. A, Reducing SDS-gels showing time-dependent degradation of biotinylated gp120 by MAb H10 (100 nM). Incubation times in lane 1-5: 2, 10, 30, 60 and 180 min, respectively. Streptavidin-peroxidase staining Coomassie stained lane 6 shows digestion of gp120 by 50 nM MAb. N-terminal sequencing of products identified the indicated scissile bonds as in ref 9. The 55 kD product corresponds to the N terminal gp120 fragment. B, Inhibition of MAb H10 gp120 cleavage by sCD4 but not an irrelevant protein (OVA, ovalbumin) indicated by reduced depletion of the intact gp120 band. MAb, 3 μg/ml; gp120, 100 nM; ovalbumin and sCD4, 1.1 μM.

FIG. 23. Polyclonal serum Ab binding titers in monkeys immunized with E-immunogens determined by ELISA. A, Example IgG and IgM binding titers for BSA-E-416-433 of sera from a monkey immunized with KLH-E-416-433 in alum and E-gp120 in Ribi. B, IgG and IgM binding titers for E-gp120 of the sera show in from panel A. C, Example IgG and IgM binding titers for E-gp120 of sera from a monkey immunized with control KLH followed by E-gp120 in Ribi. Titer is the serum dilution giving A490 of 0.2. Arrows show immunogen administration.

FIG. 24. Low level HIV neutralization by serum from a monkey immunized with KLH-E-416-433. Shown are percent HIV neutralization values for tissue culture well replicates receiving serum from a nonimmunized monkey KLH-E-416-433 immunized monkey (day 70 serum, immunization as shown in FIG. 23A). Each symbol represents one replicate. P value from Student's t-test. Strain, subtype C, CCR5 dependent strain 97ZA009; host: PBMCs.

Table 4. Neutralization of HIV by serum from a monkey immunized with KLH-E-416-433 followed by E-gp120 (day 176 serum, immunization as shown in FIG. 23). PBMC host cells. Data are from plots of varying serum dilution versus neutralization. Titer is defined as the serum dilution showing 50% neutralization (ID50).

FIG. 25. Neutralization of HIV by serum from monkey immunized with E-gp120 alone (day 175 serum, immunization as shown in FIG. 23). Serum dilution, 1:20. Strain, subtype C, CCR5 dependent strain 97ZA009; host: PBMCs.

FIG. 26. In vitro and in vivo functional activity of MAb IgG 3A5 raised from E-gp120 immunization. A, Neutralization of HIV by MAb IgG 3A5 raised by E-gp120 immunization. Strain, subtype C, CCR5 dependent strain 97ZA009 and subtype B, CCR5 dependent SHIV_{SF162P3}; host, PBMCs. B, Viral loads in SHIV-challenged macaques 7 days post-infusion of anti-E-gp120 MAb 3A5 or PBS (150 mg MAb i.v., SHIV_{SF162P3} i.v.). Each symbol represents data from one animal.

FIG. 27. Tests of IE-gp120 immunogenicity and protection against SHIV challenge. The protocol in Panel A and Panel B differs in the length of time between the final IE-120 booster and SHIV challenge. In Panel B, the Ab titers will be allowed to decrease to low levels prior to SHIV challenge. This is designed to test whether the IE-gp120 induces B cell memory that is stimulated sufficiently by SHIV to protect against infection.

Table 5. Exemplary carrier and adjuvant combinations.

FIG. 28. Hydrocarbon stapled immunogens: A, Axial helical wheel projection of the 18 residues 416-433 sequence (numbered 1-18) showing the desired hydrocarbon link (red) to stabilize helicity of the CD4 binding 425-430 region (residues 10-15 in the wheel). CD4-binding residues shown in blue in the linear sequence (bottom). B, β sheet conformation of the 416-433 region in the crystal structure of gp120 (PDB 2B4C) showing the 6-carbon crosslink between positions 423 and 434 in red. C, Structure of α-alkenyl alanines (left) introduced into the peptide at the indicated positions and the cross-link (right).

FIG. 29. Improved 421-433 CD4BS conformation due to insertion of electrophilic phosphonate groups into peptide 416-433. A, Plotted are sCD4 binding values computed as percent of binding observed using KLH-E-416-433 (A490, 0.4). sCD4, 5 μg/mL. B, Plotted are I₁₉₋₂₁ IgA binding values computed as percent of binding observed using KLH-E-416-433 (A490, 0.5). I₁₉₋₂₁ IgA from patients with HIV infection for 19-21 years, 100 μg/mL. C, Plotted are MAb YZ23 binding values computed as percent of binding observed using KLH-E-416-433 (A490, 0.6). MAb YZ23 raised by immunization with E-120, 6 μg/mL. Values are mean±s.d. from duplicates.

FIG. 30. Improved 421-433 CD4BS conformation due to elongation of peptide

E-421-433. A, Soluble CD4 reactivity of the immobilized electrophilic 421-433 CD4BS sequence mimetics, KLH-E-416-433, KLH-E-414-439 and KLH-E-421-433 (see FIGS. 3 and 4 for structures). sCD4 binding determined using rabbit anti-CD4 Ab in the presence of 100 μM E-hapten 1. KLH-E-VIP, an irrelevant electrophilic peptide described in ref 122. B, Plotted are sCD4 binding values computed as percent of binding observed using KLH-E-414-439 (A490, 1.3). sCD4, 20 μg/mL sCD4. B, Plotted are I₁₉₋₂₁ IgA binding values computed as percent of binding observed using KLH-E-414-439 (A490, 0.7). I₁₉₋₂₁ IgA from patients with HIV infection for 19-21 years, 100 μg/mL. C, Plotted are scFv JL427 binding values computed as percent of binding observed using KLH-E-416-433 (A490, 1.0). scFv JL427 isolated from a lupus phagemid library, 3 μg/mL. Values are mean±s.d. from duplicates.

FIG. 31. E-414-439 induces Abs to the neutralization-relevant 421-433 CD4BS conformation more effectively than its non-electrophilic counterpart peptide. A, Plotted are values of HIV neutralization at increasing dilution of pooled sera from mice immunized with KLH-E-414-439 or KLH-NE-414-439. X-axis data represents binding of BSA-E-414-439 or BSA-NE-4-414-439 at the serum dilutions tested in the neutralization assay. Binding was measured using sera diluted sufficiently to fall in the linear range of the ELISA assays. B, Differing binding specificity of murine serum Abs obtained by immunization with KLH-E-414-439 or KLH-NE-414-439. Plotted are values of specificity index computed as (E-gp120 IgG binding titer)/(BSA-E-414-439 IgG binding titer) or (E-gp120 IgG binding titer)/(BSA-NE-414-439 IgG binding titer). Binding titer for BSA-E-414-439 or BSA-NE-414-439 were respectively, 1:56, 198 and 1:921,405.

Table 6. Fortuitously improved recognition of Ala mutants of the 421-433 CD4BS sequence at P417, Q422, E429 by pooled IgA patients with HIV infection for 19-21 years (I₁₉₋₂₁) and murine MAb 2G9 raised by immunization with KLH-E-416-433. Values are ratios of wildtype peptide binding/mutant peptide binding computed as in Table 1.

FIG. 32. Design of binary epitope E-immunogen. A, An immunogen containing a second epitope N terminal to the 421-433 epitope mimetic (epitope 2) will induce memory cells expressing binary epitope-reactive BCRs. B, Binding of epitope 2 expressed by HIV at the BCR CDRs will generate a stimulatory signal that overcomes downregulation due to FR binding of the viral 421-433 epitope.

### DETAILED DESCRIPTION OF THE INVENTION

Production of antigen-specific antibodies (Abs) by adaptively differentiated B lymphocytes is central to protection against microbial infections. The HIV surface is studded with noncovalently associated trimeric gp120-gp41 complexes that have been the targets of numerous vaccine trials with no or little evidence for vaccine efficacy. The problem is the absence of structurally conserved coat protein epitopes essential for the viral life cycle that can induce a sufficient adaptive Ab response. Most anti-HIV Abs bind the mutable, gp120 variable (V) domain epitopes. The virus expresses very few neutralizing epitopes suitable for vaccine targeting.¹⁵ The binding site for CD4 on gp120 (CD4BS) is a conformational determinant^{10,16} containing discrete regions with mostly conserved structure across Group M HIV strains. HIV initiates infection by binding its primary host cell receptor CD4. The CD4BS is vulnerable to host immunity, and rare monoclonal anti-CD4BS Abs with broad neutralizing activity have been cloned from HIV-infected patients.^{15,17} However, the CD4BS is poorly immunogenic, and the serum of HIV infected patients contains little or no neutralizing activity attributable to anti-CD4BS Abs.

Abs similar to these MAbs are not found at appreciable levels in polyclonal Ab preparations. An HIV vaccine can be effective only if it induces robust polyclonal Abs in biological fluids with the ability to neutralize genetically diverse HIV strains. Several competing hypotheses have been proposed to explain the absence of neutralizing anti-CD4BS Abs in various vaccine strategies. Classical adaptive antibody responses involve very weak antigen binding germline B cell receptors (BCRs; Abs expressed on the cell surface), followed by antigen driven selection of mutant Ab complementarity determining regions (CDRs) that bind antigen with improved affinity. The deficient neutralizing Ab response is suggested to derive from an unusually low affinity of the vulnerable gp120 epitopes for the germline BCRs.¹⁸ Conversely, the extreme density of CDR mutations in a rare anti-gp120 neutralizing Ab prompted the suggestion that B cells are intrinsically unable to mount a sufficiently mutated Ab response.¹⁹ The CD4BS is a conformationally flexible determinant thought to undergo structural transitions upon dissociation of trimeric gp120 into its monomeric form.²⁰ Individual epitopes within the CD4BS are not equally suited for Ab targeting. Abs that bind the CD4BS of monomeric gp120 but do not neutralize HIV have been described.^{21,22} The monoclonal Ab b12 binds an epitope overlapping the CD4BS segment located in the gp120 outer domain and expresses comparatively broad neutralizing activity directed against genetically diverse HIV strains.²² An immunogen reverse engineered to present conformational complementarity with the antigen binding site of b12 did not induce neutralizing Abs.²³ Similarly, genetically engineered oligomeric gp120 did not induce broadly neutralizing Abs.²⁴

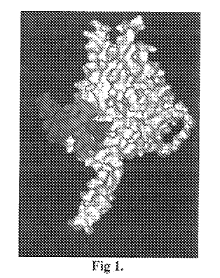

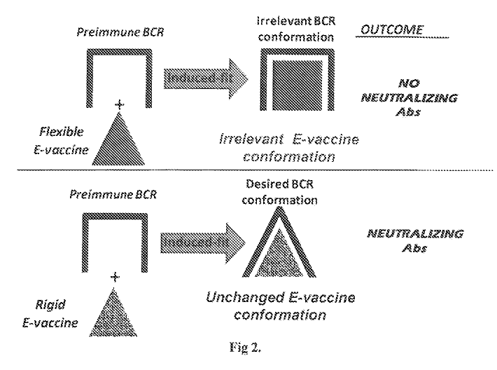

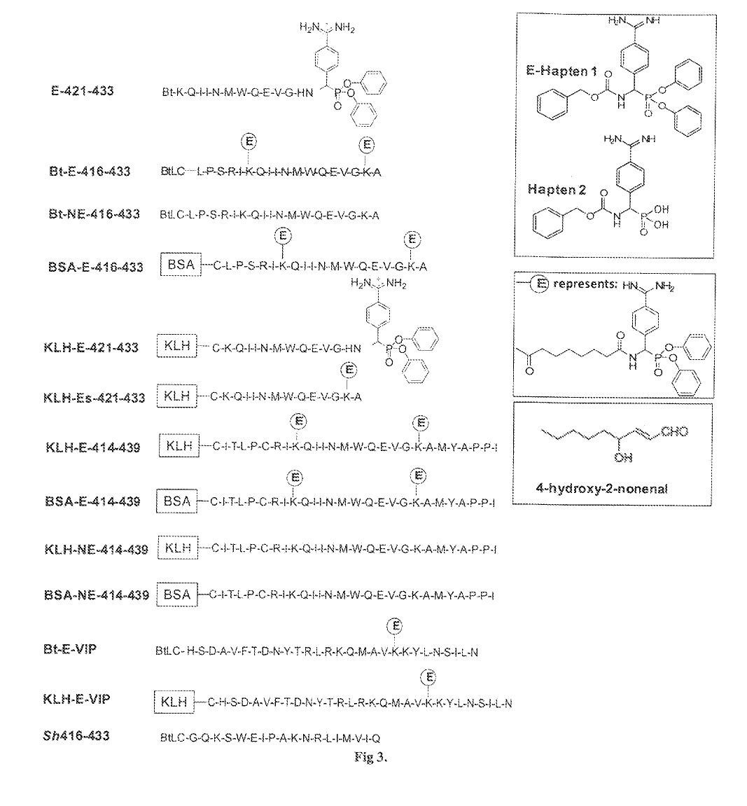

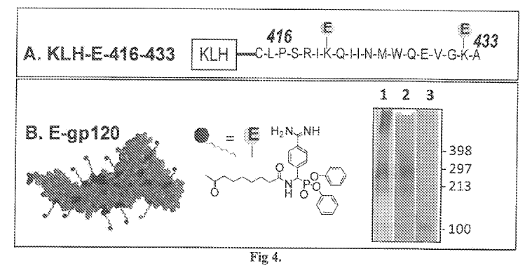

Our approach to HIV neutralization is based on targeting the core of the CD4BS, the region composed of residues 421-433 that provides critical contacts necessary for high affinity CD4 recognition.^{2,9,10,16} An ample area of 421-433 CD4BS sequence is exposed for interactions with Abs on the gp120 surface according to crystallographyl^{6,25-30} (FIG. 1A). This region is not recognized by the anti-CD4BS monoclonal Ab (MAb) b12. In addition to its role in CD4 binding, the 421-433 CD4BS sequence overlaps the B cell superantigen determinant of gp120 (120-SAg).^{31,32} Microbial superantigens are recognized by Ab variable domains (V domains) produced with no requirement for prior microbial infection.³³⁻³⁶ The binding occurs mostly at structural elements in the framework regions (FRs) located outside the classical antigen binding site formed by the CDRs.³⁴⁻³⁶ The preimmune B cell repertoire is enormous, about 10¹³ unique Abs generated by pairing of individual V_{L}/V_{H} domains encoded by ˜150 distinct V, D and J germline genes. Structural diversity of the repertoire is “constitutive” or “innate” in the sense that the diversity-generating V-(D)-J gene recombination and combinatorial pairing steps occur prior to arrival of the antigen on the scene. As the constitutively produced Abs are highly divergent structurally, the existence of distinct subsets with differing functional activities is likely. Fine structural details underlying high-level recognition of the 421-433 CD4BS sequence by individual constitutive Abs have not been elucidated, but broad guidelines are available. V_{H}3-family Abs display greatest binding to the CD4BS sequence-containing gp120-SAg determinant.³⁴ However, the FRs of various V-gene families bear substantial sequence identity with each other. Weak binding of 421-433 CD4BS sequence by non-V_{H}3 Abs and free Ab light chain subunits is reported.^{9,37} Other microbial superantigenic epitopes are recognized preferentially by V_{L} domain FRs, e.g., the light chain binding superantigen Protein L from P. magnus.

Only limited information is available about the functional importance of the constitutive Abs. As the 421-433 CD4BS sequence component of the gp120-SAg is essential for HIV infection, the Abs may protect against HIV infection if present in sufficient amounts. Indeed, elevated blood levels of 120-SAg binding Abs in non-infected humans are correlated with reduced risk for contracting HIV infection.³⁸ However, sera and purified IgG preparations from the blood of non-infected humans do not neutralize HIV in tissue culture appreciably,³⁹ raising doubt whether the constitutive Abs detected based on binding of purified gp120 and synthetic peptides as antigens can recognize the 421-433 CD4BS sequence on the viral surface with sufficient strength. We previously reported modest HIV neutralization by polyclonal salivary IgA preparations attributed to the 421-433 CD4BS sequence-specific subset of Abs that catalyzed the cleavage of gp120.³⁹ Superior HIV neutralization by catalytic IgAs is consistent with the expectation of increased potency by reuse of a single catalyst molecule for inactivating multiple gp120 molecules. Importantly, polyclonal Abs from blood and saliva examined in these studies are mixtures of diverse Abs, and the strength of HIV neutralization by the polyclonal mixtures will underestimate the neutralizing activity of individual Abs to the 421-433 CD4BS sequence represented at low levels in the mixtures.

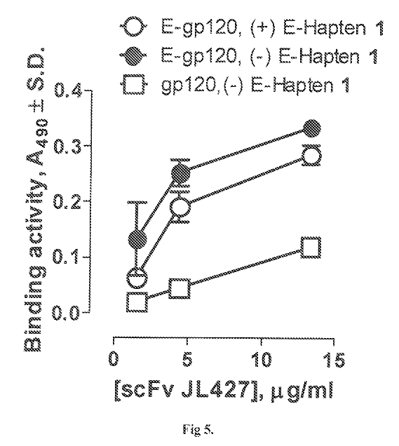

If the individual Abs to the 421-433 CD4BS sequence produced by non-infected humans can neutralize HIV with sufficient potency and breadth, tapping the innate Ab repertoire is a potential route to HIV vaccination. The feasibility of this approach depends on overcoming physiological restrictions on amplifying the constitutive Ab subset to a superantigenic epitope. Superantigen binding at the FRs of B cell receptors (BCRs; Abs expressed on the cell surface) generally fails to drive the adaptive differentiation of B cells into plasma cells producing class-switched Abs.⁴° This is exemplified by the rare production of Abs to the 421-433 CD4BS sequence by animals immunized with gp120⁴¹ and HIV-infected humans.⁴² However, the restriction on producing the Abs is leaky, and certain circumstances supporting an amplified constitutive Ab production have been identified. First, sera from patients with systemic lupus erythematosus contain increased binding Abs to the 421-433 CD4BS sequence,⁴³ and a binding Ab fragment with this specificity and HIV neutralizing activity was isolated from the lupus repertoire.^{44,45} Second, we reported IgA specific for the 421-433 CD4BS sequence class Abs with potent and broad HIV neutralizing activity that appeared in blood after prolonged HIV infection over two decades.² Third, a strategy involving covalent binding of electrophilic immunogens to the naturally-occurring nucleophilic sites of BCRs has enabled the high frequency induction of broadly neutralizing murine monoclonal Abs with specificity for the 421-433 CD4BS sequence.¹² An overriding advantage of the approach is the potentially correct specificity of the constitutive Ab FRs for the 421-433 CD4BS sequence. Even immunogens that approximate the native 421-433 CD4BS conformation imperfectly can potentially induce amplification of the CD4BS⁻ specific Ab FRs by the induced-fit mechanism (FIG. 2, Bottom). In contrast, the classical Ab response to an immunogen expressing an imperfect conformation of the 421-433 CD4BS sequence initiated by the poorly binding germline CDRs might induce non-neutralizing Abs directed to an irrelevant conformation of the 421-433 CD4BS sequence (FIG. 2, Top).

### Examples of the Invention Embodiments

The present invention overcomes the prior limitations of the art, by employing covalent immunization techniques. The invention provides improved synthetic electrophilic immunogens for HIV vaccination based on the conserved sequence of the 421-433 CD4BS sequence, amino acid nos. 421-433, of HIV gp120 that are effective in inducing the production of HIV neutralizing Abs in mammals, including humans. The probes, control reagents and improved immunogens are shown in FIGS. 3 and 4. The invention also provides improved monoclonal and polyclonal Abs raised by immunization with the electrophilic immunogens that can be used for therapy of the infection. The improvements have been possible because of identification of immunogens that approximate better the conformation of the native 421-433 CD4BS sequence expressed on the viral surface. The invention is distinguished from other vaccine approaches by the principle of recruiting and improving the innate, pre-existing ability of Abs to neutralize HIV that recognize the 421-433 CD4BS sequence at their FRs. Novel methods to identify the improved immunogens using novel template-based screening techniques involving immunogen binding by the innate Abs are disclosed. The invention also describes improved carriers and adjuvants that are uniquely formulated to facilitate the innate HIV neutralizing activity of Abs.

The invention discloses the discovery of deficient IgM→IgG class-switching as the cause of the absent neutralizing Ab response to previously tested vaccine candidate and rectification of this deficiency by immunizations with analogs of gp120, intact HIV and the 416-433 synthetic peptide containing electrophilic phosphonate groups (respectively, E-gp120, E-HIV and E-416-433). The electrophile binds covalently to the nucleophilic sites of secreted preimmune Abs⁵ and BCRs.⁴⁶ Each of U.S. Pat. Nos. 6,235,714; 6,855,528; 6,855,804; and 7,524,663 is incorporated by reference herein. These immunogens supported production of class-switched Abs to the 421-433 CD4BS sequence. Neutralizing MAbs raised by immunization with E-gp120 displayed enhanced nucleophilic activity^{6,13} and binary recognition of the 421-433 CD4BS epitope at the FRs and a second epitope at the CDRS.¹² This suggested that the down-regulatory effect of noncovalent 421-433 epitope binding at the FRs can be overcome by: (a) The highly energetic covalent binding of the immunogens to B cells; and (b) simultaneous stimulatory binding of a second epitope at the CDRs. The findings indicated covalent immunization as a viable strategy for inducing broadly neutralizing anti-CD4BS Abs.

Various immunogens with further improvements of conformation are disclosed. Their development entailed maneuvers that rigidify the 421-433 CD4BS peptide sequence alter its three-dimensional folding, including insertion of the electrophilic group, lengthening the sequence by inserting additional amino acids on its flanks, introducing mutations in the sequence and attaching the sequence to a carrier protein molecule that facilitates its folding into a native conformation.

The invention also discloses the discovery of improved conformation of the 421-433 CD4BS expressed by E-gp120 compared to gp120 (FIG. 5). In addition, the poorly immunogenic 421-433 CD4BS sequence of gp120 was converted to an immunodominant epitope by insertion of electrophilic groups into gp120. The majority of Abs induced by E-120 were directed at the 421-433CD4BS sequence. The invention also discloses subsets of E-120 variants expressing improved conformational states of the 421-433CD4BS sequence and other epitopes, and the use of the improved E-120 variants for inducing a neutralizing Ab response to the 421-433 CD4BS sequence.

Intact E-HIV expresses the minimally perturbed, native conformational state of the 421-433 CD4BS sequence. The invention discloses subsets of E-HIV variants expressing improved conformational states of the 421-433CD4BS sequence and other epitopes, and the use of the improved E-HIV variants for inducing a neutralizing Ab response to the 421-433 CD4BS sequence.

The invention discloses focusing of the Ab response at the CD4BS using an example electrophilic peptide immunogen that itself binds CD4 (E-416-433).^{2,12} Immunization of mice and rabbits with E-416-433 conjugated to a carrier protein induced polyclonal Abs that neutralized genetically diverse HIV strains. The carrier protein is an important factor in the invention, as it provides a microenvironment permitting proper folding of the E-416-433 peptide. Consistent with the electrophilic character of the immunogen, the Abs displayed robust catalytic activity.

In macaques, a common primate model for the human immune system, immunization with E-416-413 induced low level neutralizing Abs, sequential immunization with E-416-433 and E-120 induced higher level Abs, and immunization with E-120 also induced the neutralizing Abs. To the inventors' knowledge, the electrophilic immunogens are the first vaccine candidate that induces polyclonal neutralizing Ab responses to a conserved HIV site.

Covalent Vaccination.

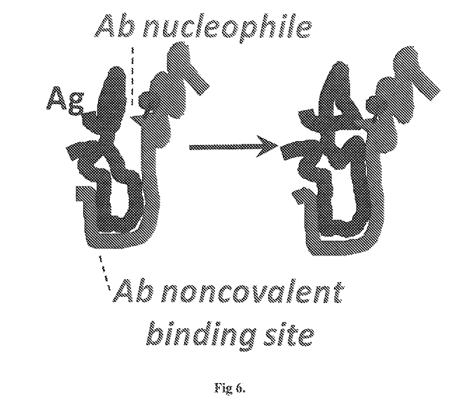

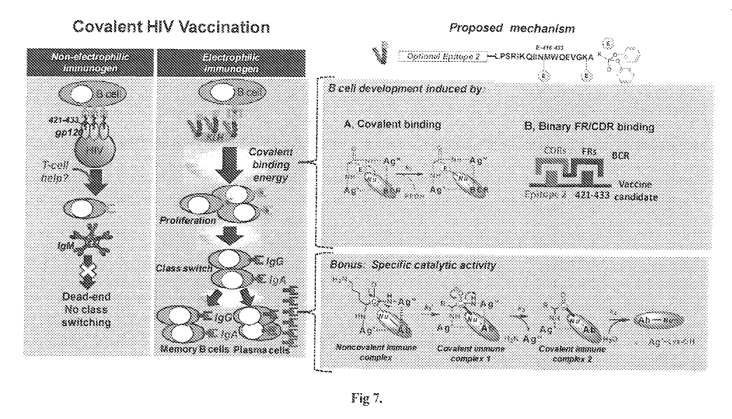

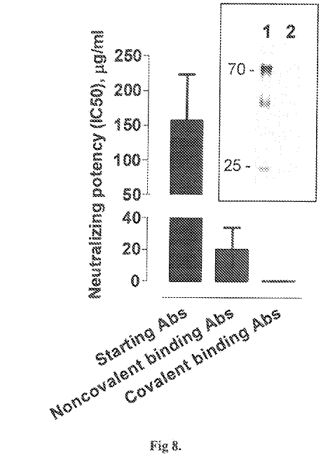

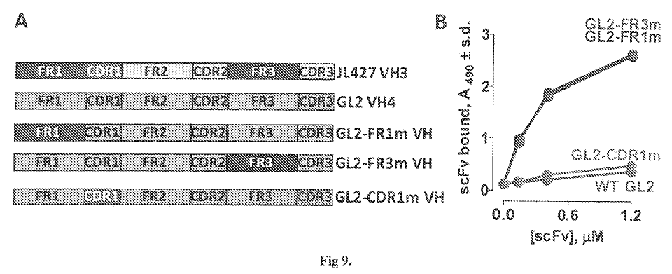

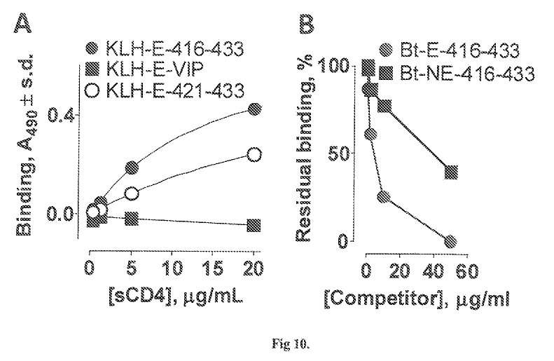

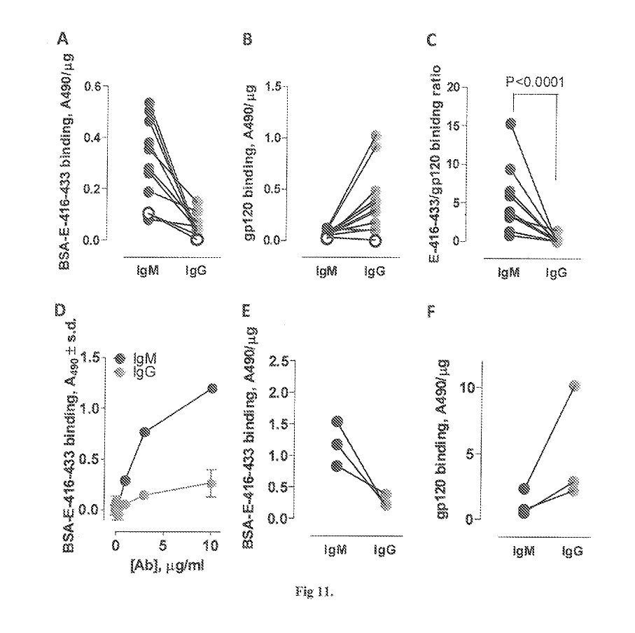

Our invention relies on the covalent binding of immunogens containing electrophilic phosphonate groups to nucleophilic Ab sites as the basis for potentially effective HIV vaccination.¹² Such nucleophilic sites were originally identified in enzymes of the serine protease family as triads of Ser(Thr)-His-Asp(Glu) residues in which the activated nucleophilic residue (Ser/Thr) forms a covalent intermediate with weakly electrophilic carbonyl group of the substrate. Abs recapitulate this catalytic mechanism, an example of convergent molecular evolution.^{39,47-49} The strongly electrophilic phosphonate group was incorporated into polypeptide immunogens in the present invention. This enables covalent immunogen binding to nucleophilic BCRs coordinated with specific noncovalent binding of the peptide epitope.^{5,46} The electrophilic immunogens are designed based on the split-site model, which states that distinct Ab subsites are responsible for initial noncovalent binding and the ensuing nucleophilic attack on the electrophilic carbonyl group of gp120 (FIG. 6).⁵⁻⁸

The reversible binding and catalytic properties of preimmune Abs to the 421-433 CD4BS sequence may provide some level of innate protection against transmission of HIV infection.^{38,39} However, any such protection comes at a heavy cost. Stimulatory antigens binding at the Ab CDRs drives B cell clonal selection. By contrast, SAg binding at the FRs is thought to downregulate B cell differentiation.⁵⁰ An impaired adaptive B cell response to the 421-433 epitope is evident from the rare production of Abs that bind peptides spanning this region in HIV infected patients⁴² and mice immunized with purified gp120 (0.005% of 140,000 MAb clones tested).⁴¹ We discovered that an impaired IgM→IgG/IgA class switching is the central defect in the adaptive immune response to the 421-433 CD4BS sequence.

Central points in the covalent vaccination approach are:

a. Covalent Binding. The highly energetic covalent reaction is hypothesized to induce favorable B cell differentiation instead of B cell downregulation due to noncovalent SAg binding (FIG. 7). In the present invention, we discovered that covalent immunization with electrophilic immunogens repairs defective IgM→IgG class switching characteristic of the physiological anti-421-433 Ab response, permitting synthesis of powerful neutralizing Abs. We also discuss improved binary epitope immunogens that may overcome the downregulatory effect of FR binding by the 421-433 epitope due to simultaneous stimulatory engagement of a second epitope by the CDRs.¹²

b. Vaccine Conformation. To induce neutralizing Abs, the immunogen must mimic the native epitope conformation expressed on the HIV surface. One vaccine prototype of the invention, the electrophilic analog of resides 416-433 (E-416-433), binds specifically to CD4, verifying that the 421-433 region is expressed in a native CD4-binding conformation. E-416-433 is also recognized specifically by broadly neutralizing Abs from survivors of HIV infection. Most importantly, the present invention discloses induction of the synthesis of HIV neutralizing polyclonal and monoclonal Abs in animals by E-416-433 conjugated to the carrier protein keyhole limpet hemocyanin (KLH). Although not appreciated widely, the carrier protein influences the conformation of conjugated peptides profoundly. The pitfall of small peptide immunogens is their ability to assume alternate conformations in varying microenvironments. This is exemplified by our report that Abs raised by immunization with the 421-436 peptide conjugated to different polypeptides displayed differing binding specificity.⁵¹ If the peptide folds into a non-native conformation, the immune system will not amplify or improve the innate epitope recognition capability of the Ab FRs. The carrier protein is important because it provides the local microenvironment composed of neighboring amino acids that induce flexible peptides into distinct conformations. E-416-433 also contains the LPSR1 residues at the N terminus, which stabilizes the conformation of the 421-433 region.^{52,53} The side chain chemical modification of the peptide with phosphonate groups is also a significant feature as it improves binding to CD4 and Abs by imparting rigidity to the epitope mimetic. Since E-416-433 binds CD4 ˜100-fold more strongly than previously tested 421-433 region peptides, it is predicted to induce a superior neutralizing Ab response. Mutagenesis studies showed that key amino acids necessary for the CD4 binding function of E-416-433 are also essential recognition elements of the Ab epitope. Previous studies on Abs to peptide immunogens containing part or all of the 421-433 region but without the essential features of KLH conjugated E-416-433 conjugated KLH failed to induce sufficiently neutralizing Abs to the 421-433 CD4BS region.^{42,54-56} Importantly, as the previously tested vaccine candidates did not mimic the native 421-433 CD4BS conformation sufficiently, they induced the classical CDR-based response. The CDR-based response results in production of non-neutralizing Abs because of induced-fit considerations shown in FIG. 2. In contrast, the immunogens of the present invention recruit and improve adaptively the innate FR-based 421-433 CD4BS sequence recognition site. Abs that use the innate, FR-based site for recognition of the native 421-433 CD4BS sequence neutralize HIV potently (FIGS. 8 and 9). Consequently, the present invention embodies the novel principle of amplifying innate immunity for vaccination against HIV.

In addition to the carrier protein conjugated peptides, the invention discloses improved variants of full-length E-gp120 and E-HIV that offer the advantage of improved 421-433 CD4BS sequence conformation suitable to amplify the FR-based recognition site of Abs. Yet another advantage of the improved E-gp120 and E-HIV variants is the availability of additional epitopes for binding the CDRs. A second epitope that binds the CDRs simultaneously with binding of the 421-433 CD4BS sequence to the FR-based sites is helpful, because such a binary binding reaction overcomes the negative cellular signaling associated with binding of the FRs alone (FIG. 7).

c. Catalytic Activity. Covalent immunization offers the bonus of strengthened Ab nucleophilic reactivity by virtue of adaptive selection of BCRs that bind the electrophile most strongly.^{6,13,57} In turn, the enhanced nucleophilicity improves HIV inactivation as follows (FIG. 7; lower pink panel). First, specific pairing of the Ab nucleophile with the weakly electrophilic carbonyls of gp120 results in formation of stable immune complexes with covalent character. This can occur because water attack on the covalent complex and completion of the catalytic reaction cycle is very slow. Covalently binding Abs were induced by immunization with the electrophilic analogs of full-length gp120 as well as a gp120 V3 peptide.^{13,57} Unlike reversible immune complexes, the covalent immune complexes do not dissociate readily, resulting in enhanced neutralization potency.⁵⁷ Second, if the Ab contains accessory groups supporting water attack on the covalent acyl-Ab complex, catalytic gp120 cleavage occurs. Abs raised by immunization with E-416-433 hydrolyzed gp120 rapidly, indicating the ability of the peptide immunogen to induce Ab combining sites containing structural elements that facilitate water attack on the acyl-Ab complex. A single catalytic Ab molecule is reused to cleave thousands of gp120 molecules over its biological half-life in blood (1-3 weeks). Therefore, the neutralization potency is enhanced compared to reversibly-binding Abs.^{39,58}

Preclinical Ab Protection Assays.

Tissue culture infection models are indispensable to assess the potency and breadth of neutralizing activity of Abs induced by candidate vaccines.⁵⁹ CCR5-dependent primary strains are substantially more resistant to most Abs directed to the HIV proteins compared to lab-adapted strains.⁶⁰ Abs to the 421-433 CD4BS epitope neutralize the most ‘difficult-to neutralize strains’ in the classical peripheral blood mononuclear cell (PBMC)/clinical isolate infection assay. This assay is the closest available tissue culture model for the natural infection process. Engineered reporter cell lines and pseudovirions have been developed for convenient analysis of large numbers of Ab samples, e.g., the TZM-B1 cell line/pseudovirion assay.⁶¹ Several publications have noted discrepant neutralizing activities of anti-HIV MAbs in the PBMC/clinical isolate and TZM-B1/pseudovirion assays.^{22,62,63} Excessive expression of the coreceptor CCR5 on TZM-B1 cells compared to PBMCs is cited as a potential reason for discrepancies (˜1000-fold difference of CCR5 levels).⁶⁴ The conformational flexibility of gp120^{20,26,65} in differing membrane microenvironments is another variable. Variations in the conformations of the epitopes expressed by clinical HIV isolates versus pseudovirions are conceivable.

HIV infects chimpanzees transiently. The infection does not progress to AIDS. Immunization of chimpanzees with recombinant gp120 suppressed HIV viremia, but human trials of the gp120 immunogen did not reduce HIV infection risk.⁶⁶⁻⁶⁸ As the HIV and SIV envelope proteins are structurally divergent, direct testing of candidate HIV vaccines in the SW-infection model is difficult. Hybrid simian-human virus strains (SHIN) containing the HIV envelope proteins grafted into SW produce viremia in rhesus monkeys. Candidate vaccines that induced cytotoxic T cells protected monkeys from SHIV infection but did not protect humans from HIV infection.⁶⁹ The SHIV/rhesus monkey model was recently suggested to be a useful ‘gatekeeper’ to identify candidate vaccines that induce ‘better immunity’ compared to the failed immunogens.⁷⁰ One use of the SHIV-monkey model is to determine the circulating/mucosal titers of Abs needed to prevent or reduce the acute stage of infection in vivo. Moreover, the immune system of monkeys may be a good model of the human response to candidate vaccines compared to phylogenetically lower species. Therefore, monkey studies were done to validate the present invention.

The present invention developed and utilized the following electrophilic immunogens: E-120, intact E-HIV and synthetic E-peptides containing the 421-433 peptide region. Improved E-peptide and E-gp120 variants have been identified as the prototype vaccine candidates based on the properties of Abs induced by these immunogen in mice, rabbits and monkeys. The studies also resulted in unexpected findings of: (a) down-regulated adaptive immunity attributable to the SAg character of the 421-433 CD4BS epitope; and (b) upregulation of the adaptive immune response by covalent stimulation of B cells with the electrophilic immunogens.

The following findings were made, as discussed in further detail below:

Preimmune IgM and IgA from humans/experimental animals express the innate capability to recognize the 421-433 region by noncovalent means and proceed to catalyze the cleavage of gp120.

Adaptive synthesis of Abs to the 421-433 is down-regulated upon HIV infection/immunization with gp120.

Survivors of prolonged HIV infection mount a slow Ab response to the 421-433 epitope with eventual production of Abs that neutralize genetically diverse HIV strains with exceptional potency.

Covalent immunization with improved electrophilic immunogens overcomes the B cell down-regulation. The prototype vaccine E-416-433 conjugated to KLH induces the production of a focused anti-CD4BS Ab response that neutralizes diverse HIV strains.

E-gp120 and its improved variants induce Abs that neutralize diverse HIV strains.

Immunization with E-416-433 and E-120 results in improved covalent binding and catalytic hydrolysis of gp120 by Abs.

### Example 1

### E-416-433, an Improved Probe for Neutralizing ABS

FIG. 4 shows the structure of KLH-E-416-433, the prototype vaccine. Its apparent dissociation constant for specific binding to soluble CD4 (sCD4) from FIG. 10A data is 182 nM, a value >120-fold superior to previous peptide mimetics of this region.^{52,71} The longer E-416-433 probe displayed superior sCD4 binding compared to the shorter E-421-433 mimetic. Bt-E-416-433 was a superior competitive inhibitor of sCD4 binding to immobilized KLH-E-416-433 compared to the non-electrophilic mimetic Bt-NE-416-433 (FIG. 10B). Therefore, chemical modification of the peptide with the phosphonate groups induces the peptide to assume a favorable conformation. It was concluded that KLH-E-416-433 is suitable for study of naturally occurring Abs to the CD4BS and for induction of such Abs

Use of E-416-433 for Improved Detection of Preimmune ABS to 421-433 CD4BS Epitope.

The peptide component of E-peptides bind reversibly to the traditional noncovalent binding site of Abs. In addition, the electrophilic phosphonate component binds the nucleophilic sites of catalytic Abs irreversibly. We reported previously the cleavage of gp120 by catalytic IgM and IgA class Abs from humans without HIV infection (preimmune Abs) that was selectively inhibited by the short E-421-433 peptide, indicating the importance of the 421-433 region for initial noncovalent binding.^{9,39} Preimmune SIgA from saliva neutralized HIV with modest potency. The rank orders of neutralization potency and gp120 cleavage rates were the same: SIgA>serum IgA>>serum IgG.³⁹ This is consistent with more efficient virus inactivation by catalysts compared to reversibly-binding Abs.

We used the longer E-416-433 peptide to isolate highly neutralizing Abs from preimmune human and murine Abs also Enriched HIV neutralization of the epitope-specific Abs isolated from human and mouse serum by affinity chromatography on agarose-conjugated E-416-433 was evident (FIG. 8). We reported previously that covalent binding to electrophilic phosphonates predicts the magnitude of Ab catalysis.⁶ Therefore, we also tested the covalently bound Abs eluted after pyridine 2-aldoxime methiodide (PAM) cleavage of the phosphonate-Abs bonds. The covalently bound Abs displayed greater HIV neutralizing potency compared to the non-covalently bound Abs recovered by traditional acid elution. This is consistent with superior HIV neutralization due to the catalytic function.

Trace protease contamination in various catalytic Ab preparations tested by our group was ruled out as follows: Fab fragments retained the activity;⁹ the activity was not removed by denaturing gel filtration, a procedure that frees Abs of noncovalently-associated proteins;^{9,39} the Abs were purified to constant catalytic activity by sequential chromatography steps;⁹ active site titration indicated that the number of catalytic sites corresponds to the predicted number of sites;³⁹ unlike conventional proteases, the Abs hydrolyzed gp120 specifically;^{6,9,39} recombinant Abs with 421-433 CD4BS sequence binding activity hydrolyzed gp120;^{37,45} and catalytic monoclonal Abs were obtained by immunization with electrophilic gp120.⁶ We have also mapped the catalytic site of Abs by mutagenesis⁴⁷ and V_{L}-V_{H} domain shuffling.^{72,73} The catalytic site of one of our Abs was identified by crystallography.⁴⁸

Lupus patients mount Ab responses that are normally disfavored in humans with autoimmune disease. HIV infection occurs rarely in lupus patients. HIV neutralizing Ab fragments specific for the 421-433 region prepared from lupus patients without HIV infection were reported.^{37,44,45} These fragments recognize E-416-433 strongly, e.g., the V_{H}3 family single chain Fv (scFv) JL427 binds KLH-E-416-433 with K_{d} 16 nM. V_{H}3 family Abs are thought to recognize the gp120 SAg site preferentially.³⁴ We replaced the FR1, FR3 and CDR1 of the V_{H}4-family scFv clone GL2 with the corresponding V_{H}3-family scFv JL427 regions (FIG. 9A). The chimeric FR1/FR3 scFv GL2 mutants displayed increased binding of full-length E-gp120 and KLH-E-416-433 (FIG. 9B). Taken together, these studies showed: (a) small amounts of specific Abs to the 421-433 CD4BS sequence are present in the preimmune repertoire; (b) a subset of Abs catalyze the cleavage of gp120; and (c) Ab FRs are important in recognition of the 421-433 CD4BS sequence.

Use of E-416-433 for Discovery of Impaired Class Switching and Slow, Infection-Induced Adaptive AB Response.

The mature systemic Ab response is normally dominated by IgG Abs. In contrast, IgMs dominated the Ab response to BSA-E-416-433 in HIV infected patients despite prolonged infection (0.5-5 years; n=10; FIG. 11A). IgG binding to full-length gp120 exceeded the IgM binding in the same patients, indicating normal class switching of Abs to the immunodominant gp120 epitopes (FIG. 11B). These data suggest impaired IgM→IgG class-switching of Abs to the 421-433 CD4BS epitope. This conclusion is illustrated by divergence between the ratio (E-416-433 binding/gp120 binding) for the IgM versus IgG fractions (FIG. 11C). Immunizations of mice with full-length gp120 supported the conclusion of impaired epitope-specific class switching. In 3 independent repeat immunization experiments, binding of BSA-E-416-433 was consistently dominated by IgM class Abs, whereas binding of full-length gp120 was dominated by IgG class Abs (FIG. 11D,E,F). This data suggested deficient class-switching as an impediment to inducing synthesis of Abs to the 421-433 CD4BS epitope.

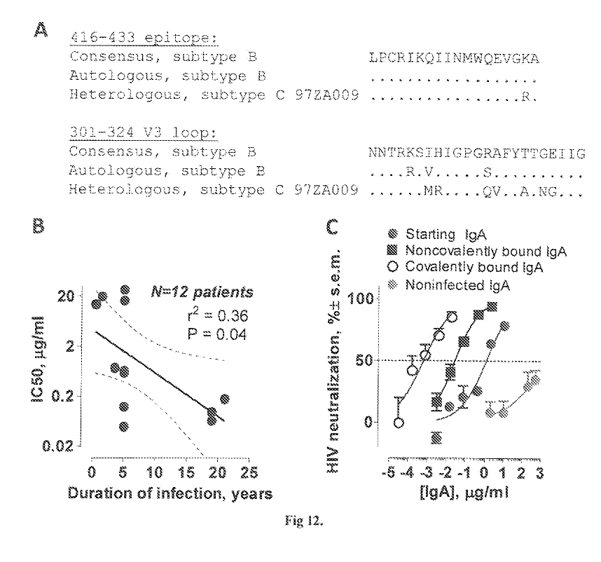

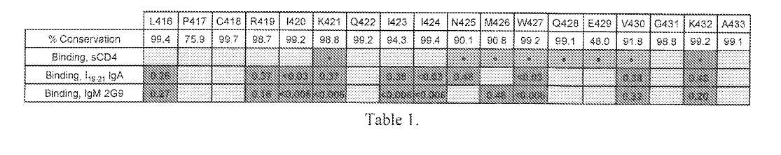

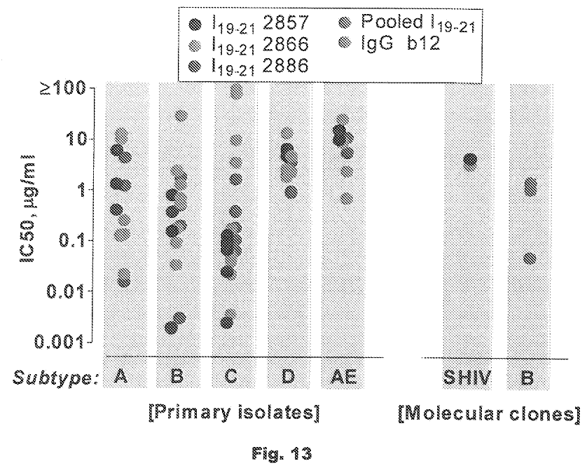

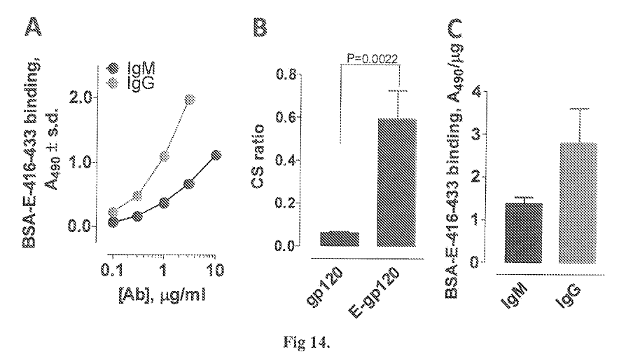

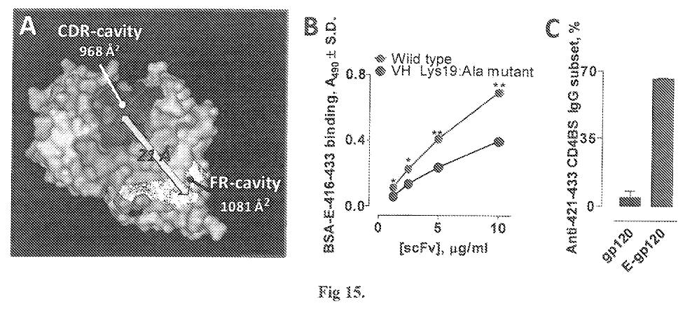

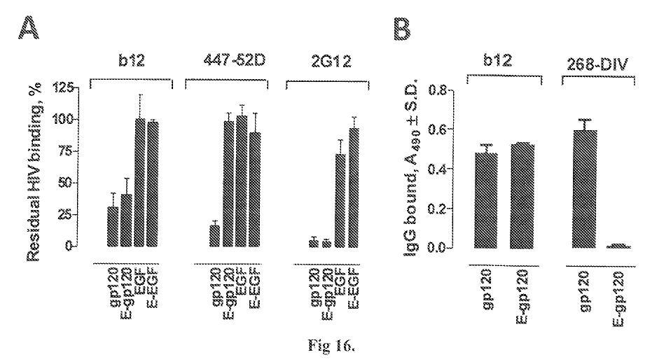

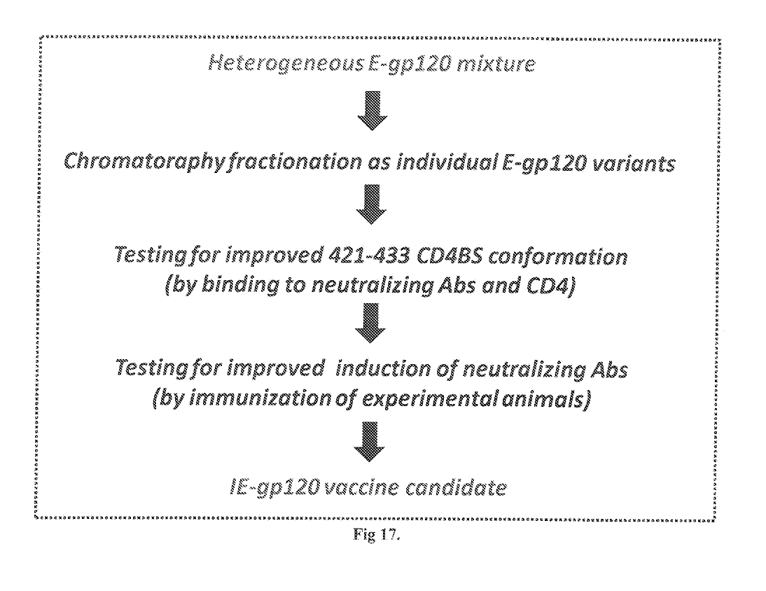

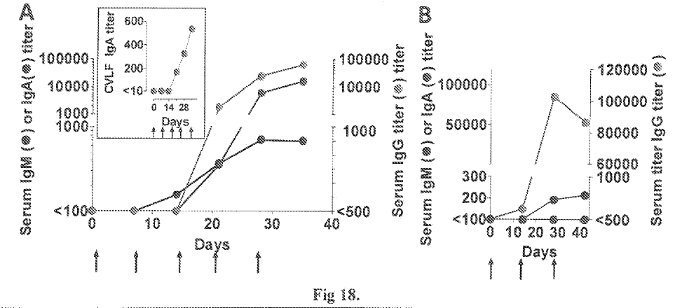

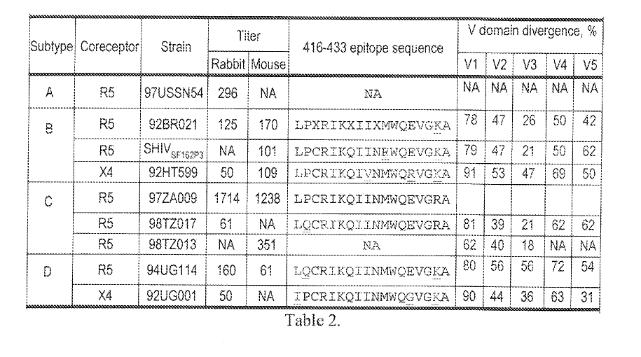

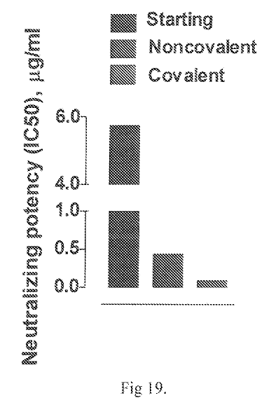

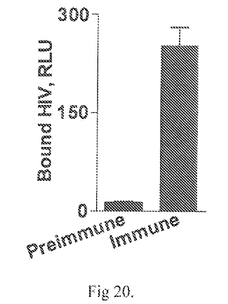

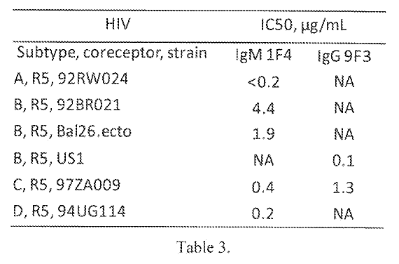

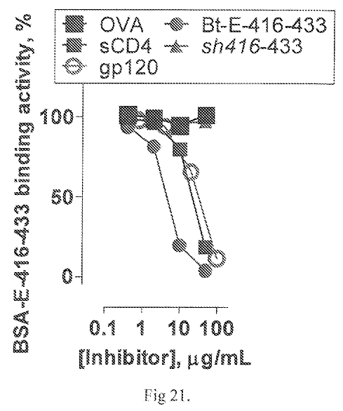

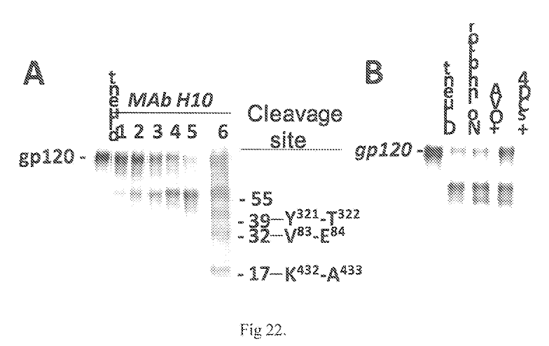

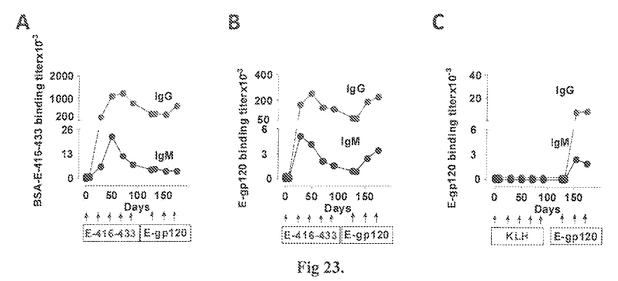

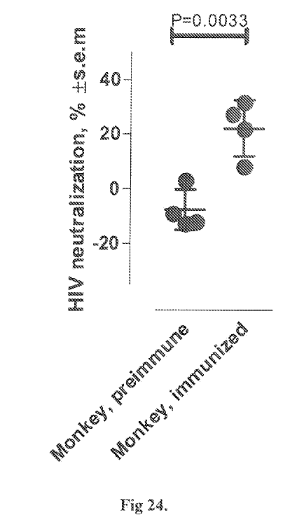

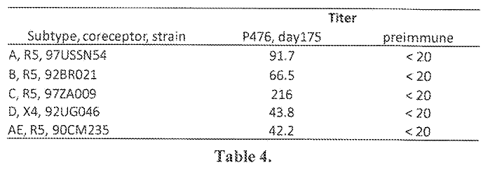

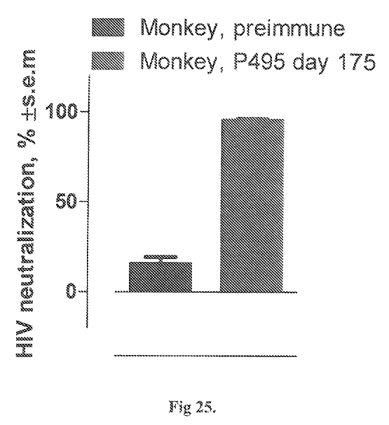

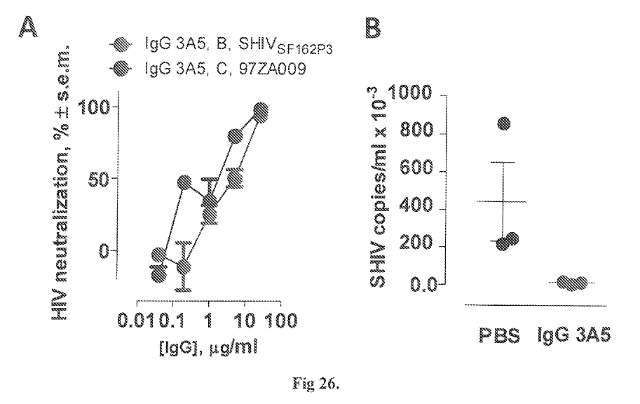

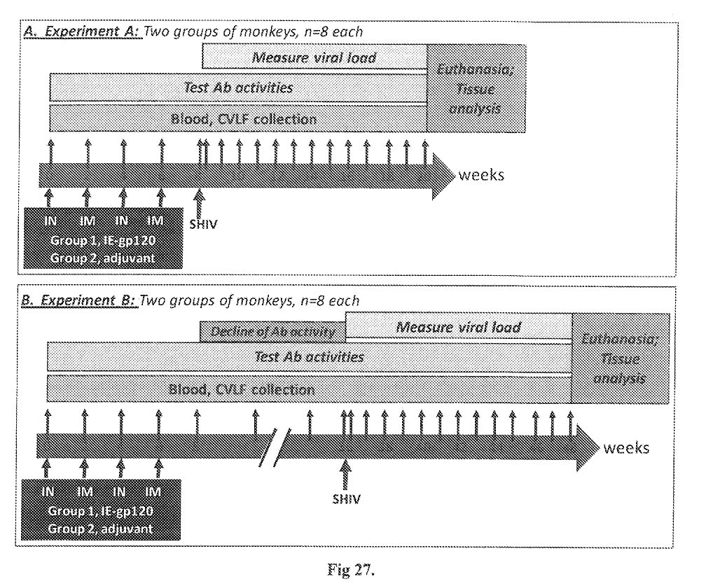

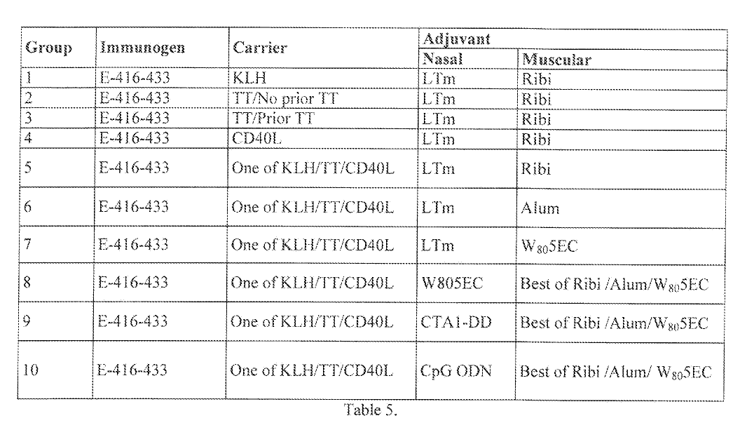

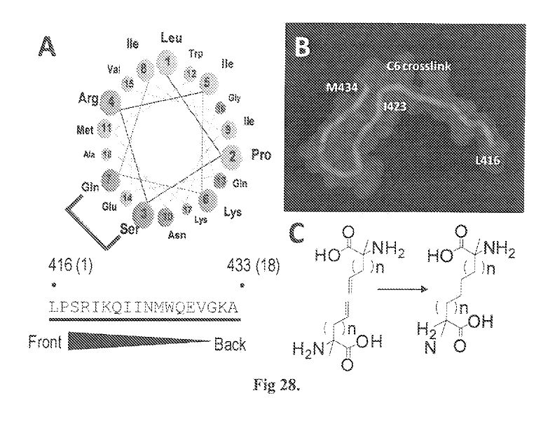

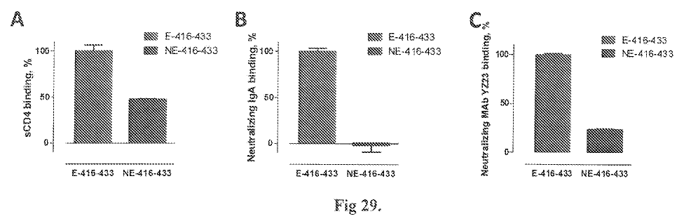

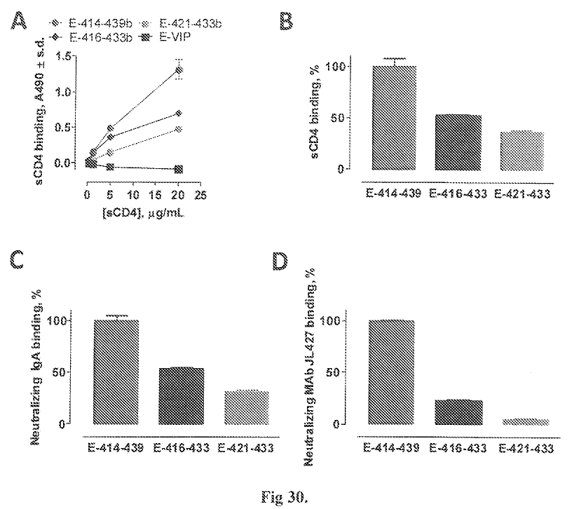

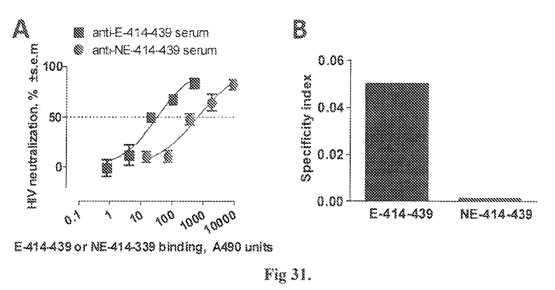

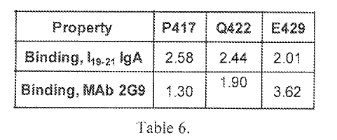

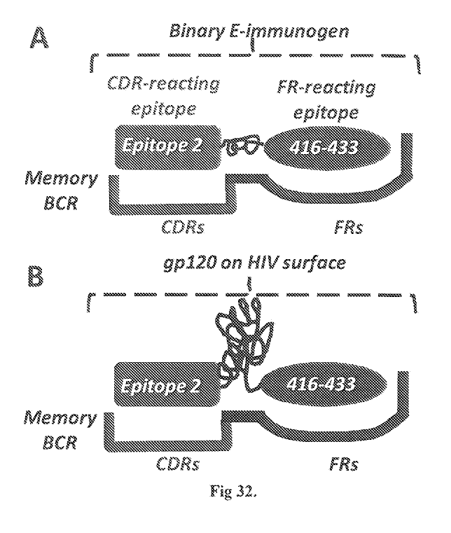

That IgA fractions from humans without HIV infection display superior HIV neutralization compared to the IgG fractions has been reported.³⁹ Therefore, the neutralization of a heterologous subtype C primary HIV isolate by purified IgA from the blood of 12 patients infected for varying durations with presumptive subtype B HIV strains (0.5-21 years, all patients from the USA; subtype B infection was confirmed for 3 patients infected for 19-21 years by sequencing the gp120 gene²) was tested. The subtype C HIV strain was selected to minimize detection of Abs to the gp120 V domains, as such Abs do not neutralize genetically heterologous HIV strains. The subtype C strain is not neutralized by murine Abs to subtype B full-length gp120. The sequences of its V domains diverge substantially from the autologous subtype B strains identified in the I₁₉₋₂₁ patients (e.g., the V3 domain epitope shown in FIG. 12A). IgA from all infected patients neutralized the subtype C virus more potently than control IgA from non-infected humans. However, contrary to the expectation of a fully matured adaptive Ab response within weeks of contracting infection, there was a significant increase of IgA neutralizing potency as a function of infection duration up to 21 years (FIG. 12B). This suggests an impaired neutralizing IgA response requiring unusually prolonged exposure to HIV.

The slow but distinct Ab response to the 421-433 CD4BS response can be interpreted favorably with respect to the prospect of therapeutic vaccination, as the response will generate CD4BS-specific memory B cells. The present invention provides immunogens that rapidly induce HIV neutralizing Abs against the CD4BS. The immunogens can be used to amplify the CD4BS-memory B cells found in HIV infected patients as a therapeutic vaccine strategy.

The functional properties of IgA from 3 survivors of prolonged infection (19-21 years; I₁₉₋₂₁ patients) has been reported.² Affinity chromatography of IgA from these patients on immobilized E-416-433 yielded epitope-specific eluates with enriched E-416-433 binding activity. The subtype C strain was neutralized by the noncovalently bound IgA obtained by acid elution and covalently bound IgA obtained by PAM elution more potently than the starting IgA (respectively, by 151-fold and 4688-fold FIG. 12C). Consistent with the neutralization data, IgA from the I₁₉₋₂₁ patients displayed increased E-416-433 binding compared to IgA from non-infected subjects. Specificity of IgA binding was evident from competitive inhibition by soluble E-416-433, full-length gp120 and sCD4, but not by control Sh416-433 peptide with shuffled sequence (GQKSWEIPAKNRLIMVIQ) or the irrelevant protein ovalbumin.² Competitive inhibition of IgA binding by 416-433 peptides containing an Ala replacement mutation at each position identified the amino acids in the epitope necessary for IgA recognition (Table 1). Of 8 residues in the epitope important for CD4 binding, 10, 11, 16 mutations at 5 resulted in reduced IgA binding. All 3 I19-21 IgA preparations neutralized all 18 strains in a panel of genetically divergent CCR5-dependent HIV strains (FIG. 13; PBMC/clinical isolate assay, details in reference 2). IgA potencies often exceeded the reference anti-CD4BS MAb b12 potency. As all strains tested were neutralized, there is no evidence that production of Abs to the CD4BS provides a selective pressure for emergence of escape mutants. Although slow to develop, the infection-induced IgAs to the 421-433 CD4BS region neutralize diverse HIV strains.

### Example 2

### Rectifying Impaired Antibody Class Switching by Covalent Immunization

The covalent binding of electrophilic phosphonates placed into our E-immunogens to B cell receptors and secreted Abs was reported previously.^{5,6,46} In the present invention, immunization with E-gp120 induced a robust IgG response to the 421-433 CD4BS epitope detected using BSA-E-416-433 as the antigen in ELISA tests (FIG. 14A). In contrast, the epitope-specific Ab response is dominated by IgM class Abs after immunization with gp120 devoid of the electrophilic groups (compare FIG. 14A with FIG. 11D). More efficient induction of class-switching by the E-gp120 immunogen was observed in 3 independent immunizations shown in FIG. 14B. To minimize any overall class switching differences in various immunizations, the data are reported as the CS ratio (CS, anti-E-416-433 Ab response/CS, overall anti-gp120 Ab response), where CS denotes the extent of class switching computed as (A490 IgG binding activity/A490 IgM binding activity). Similarly, intact HIV labeled with electrophilic phosphonate groups (E-HIV) effectively induced an IgM→IgG class switched E-416-433 binding Ab response (FIG. 14C). Control HIV without phosphonate derivitzation failed to induce detectable E-416-433 binding Abs. We concluded that covalent immunization with E-gp120 solves the problem of deficient IgM→IgG class switching of Abs to the 421-433 region.

Rectification of the deficient class switching is consistent with our report of high frequency induction of monoclonal IgGs by immunization with E-gp120.¹² Seven of 17 anti-E-gp120 MAbs displayed neutralizing activity attributable to 421-433 CD4BS epitope recognition. The MAbs neutralized genetically divergent clinical HIV isolates (n=11 strains), including all ‘difficult-to-neutralize’ strains tested (2 tier 1 strains and 3 tier 2 strains). Of the 7 neutralizing MAbs, 6 displayed binary-epitope reactivity. That is, the same MAb was able to bind two peptide epitopes that are spatially separated in the gp120 crystal structure (residues 301-311 and 421-431). Monovalent Fab and scFv fragments also displayed the binary epitope reactivity. The crystal structure of a Fab fragment indicated an antigen binding cavity formed by the CDRs flanked by another cavity formed by V_{H} FR residues previously implicated in gp120 binding by preimmune Abs (FIG. 15A).^{35,36} Site-directed mutagenesis at a residue in the FR-cavity reduced the binding of E-416-433, confirming recognition of the 421-433 CD4BS epitope at this cavity (FIG. 15B). The ratio of replacement/silent mutations exceeded the ratios anticipated for a random mutational process in the V_{H} FRs (but not the V_{L} FRs), suggesting V_{H} FR adaptive diversification. The analyses also suggested non-random V_{H} germline gene usage (but not non-random V_{L} germline usage), suggesting biased recruitment of the innate Ab repertoire. Covalent immunization with E-gp120 induces neutralizing Abs to the 421-433 CD4BS region may occur by two mechanisms: (a) The highly energetic covalent reaction with B cells; and (b) Stimulatory CDR-engagement by a second gp120 epitope occurring simultaneously with FR-engagement of the 421-433 CD4BS region.

As noted previously, Abs to the 421-433 CD4BS sequence are produced very rarely by immunization with monomeric gp120 devoid of electrophilic groups.⁴¹ E-gp120, however, present a strongly immunogenic 421-433 CD4BS epitope on its surface. Immunoadsorption of the E-gp120 immunogen-induced polyclonal Abs on the E-416-433 probe removed 66% of E-gp120 binding Abs (FIG. 15C). In contrast, there was little or no removal of gp120 binding Abs induced by gp120 immunization by the same immunoadsorption procedure. It may be concluded that inserting electrophilic phosphonates into gp120 converts the poorly immunogenic 421-433 CD4BS sequence into an immunodominant epitope.

### Example 3

### Variants of E-GP120 and E-HIV as Improved Immunogens

E-gp120 Variants

E-gp120 is not a homogeneous immunogen. It is prepared by insertion of electrophilic phosphonate groups into the side chains of surface Lys groups of gp120. It is commonly assumed that the starting monomer gp120 molecules into which the electrophiles are inserted represent a single, conformationally homogeneous population. Given the conformational heterogeneity of the CD4BS and other segments of gp120,^{26,52} this assumption may be incorrect, in which case, the conformationally distinct starting monomer gp120 molecules will give rise to conformationally distinct E-gp120 monomers. Heterogeneity is also created by the electrophile insertion step. On average, about 70% of surface accessible Lys side chains are linked chemically to the electrophile. The number of electrophiles per molecule of gp120 may be assumed to be distributed in a Gaussian fashion, with subsets of molecules containing differing numbers of the electrophiles. Moreover, insertion of the electrophile into gp120 results in intermolecular covalent bonding between the monomeric gp120 molecules, resulting in formation of various oligomeric species, including well-defined trimers and dimers (FIG. 4). The intermolecular reaction occurs by covalent bonding between the electrophilic phosphonate with an endogenous gp120 nucleophilic site guided by low affinity gp120-gp120 binding interactions.^{5,74} Note that gp120 is expressed on the surface of HIV as noncovalently self-associated trimers.

Each of the foregoing mechanisms holds potential for altering the CD4BS conformation in subtle but important ways. FIG. 5 and FIG. 16 show the differential reactivity of an oligomerized E-gp120 preparation and monomeric gp120 with various Abs. E-gp120 displays improved reactivity with Abs to the 421-433 CD4BS epitope and reduced reactivity directed to the third variable domain of gp120.

The present invention discloses the use of improved E-gp120 variant species (IE-gp120) for inducing neutralizing Abs to the CD4BS. Methods for isolating and using the IE-gp120 variant species expressing the CD4BS in a conformation resembling the native viral CD4BS are also disclosed. Improved mimicry of the native CD4BS by the immunogens can be anticipated to induce improved neutralizing Abs.

As E-120 oligomers are stably linked, fractionating various IE-gp120 species is routine (FIG. 17). One embodiment of the invention provides one or more fractions of IE-gp120, such as one or more column fractions. Fractionation may be performed by one or more of various routine methods, for example, by resolutive size, charge and/or hydrophobic HPLC methods (e.g., Superose, Mono Q and hydroxylapatite columns). In addition, affinity chromatography using immobilized neutralizing Abs or immobilized CD4 may be employed to identify the IE-gp120 species expressing the most favorable CD4BS conformation. For example, the IE-gp120 species may be isolated by binding to the highly-neutralizing scFv JL427 or IgA from patients with very prolonged HIV infection. As these Abs recognize the native conformation of the 421-433 CD4BS sequence, the procedure identifies the IE-gp120 species expressing the native 421-433 CD4BS conformation.

The IE-gp120 species may be tested for immunogenicity in experimental animals as in Example 2, Example 4-8 and Examples of Methods. The immunogenicity tests include measurement of the ability of induced Abs to bind the 421-433 CD4BS sequence, catalyze the hydrolysis of gp120 and neutralize genetically diverse HIV strains. In addition, the Abs may be capable of removing virus through certain Fc-dependent functions, for example, Ab-dependent cellular virus inhibition.⁷⁵ Therefore, the sera may also be tested for Fc-dependent viral removal using appropriate assays to determine the mechanism of Ab action.

E-HIV and E-HIV Variants

Like the E-gp120 immunnogen, the intact E-HIV immunogen described in Example 2 overcomes the problem of deficient Ab class switching and induces a class-switched Ab response directed at the 421-433 CD4BS sequence.

Another advantage of the E-HIV immunogen is the that it expresses gp120 trimers with the 421-433 CD4BS sequence expressed in its native conformation with minimal perturbation due to insertion of the electrophilic groups. Thus, E-HIV is likely to induce Abs that recognize the neutralization-relevant conformation of the 421-433 CD4BS sequence.

Once electrophilic phosphonates are incorporated into the amino acids side chains of the virally expressed gp120, intermolecular covalent bonding of the gp120 molecules composing the trimeric gp120 complexes may occur by the same mechanisms as in the case of E-gp120 covalent self-assembly, that is, by means of the covalent reaction between the electrophilic phosphonate and a naturally occurring nucleophilic amino acid of gp120.

As in the case of side chain labeling of purified gp120, varying numbers of the electrophilic group are incorporated into the side chains of virally expressed gp120, creating heterogeneity with respect to conformation. Similarly, heterogeneity with respect to the degree of covalent oligomerization of the trimeric gp120 on the viral surface is also likely.

In addition to gp120, the electrophilic groups will also be incorporated into the side chains of other proteins expressed on the viral surface.

The present invention discloses the use of E-HIV and its improved variant species (IE-HIV) for inducing neutralizing Abs to the CD4BS. Methods for isolating and using the E-HIV and IE-HIV variant species expressing the CD4BS in a conformation resembling the native viral CD4BS are also disclosed. Improved mimicry of the native CD4BS by the immunogens can be anticipated to induce improved neutralizing Abs.

E-HIV is prepared using inactivated HIV from a suitable virus strain, for example strain MN. The HIV-1 particles in the supernatants of cell culture supernatants are purified by precipitation using 1-2% polyethylene glycol (PEG) or another method such as gel filtration chromatography. This removes soluble proteins, including monomer gp120 shed from the virus. Inactivation is done using psoralen and UV light or 2-aldrithiol, methods that minimize disruption of the native surface structure of HIV-1. Insertion of electrophilic phosphonate groups into Lys side chains of surface proteins expressed on the surface of HIV particles is done essentially as described for purified E-gp120 preparation using a neutral pH buffers. The E-HIV is purified by PEG precipitation, the extent of phosphonate insertion per unit protein mass of the E-HIV is determined, and the E-HIV is tested as an immunogen in experimental animals as described in Example 1 and 3.

To obtain improved E-HIV (IE-HIV) variants expressing the minimally perturbed, native 421-433 CD4BS conformation, the E-HIV is fractionated as described for E-gp120. One embodiment of the invention provides one or more fractions of IE-HIV, such as one or more column fractions. Fractionation may be performed by one or more of various routine methods, for example, by resolutive size, charge and/or hydrophobic HPLC methods (e.g., Superose, Mono Q and hydroxylapatite columns). In addition, affinity chromatography using immobilized neutralizing Abs or immobilized CD4 may be employed to identify the IE-HIV species expressing the most favorable CD4BS conformation. For example, the IE-HIV species may be isolated by binding to the highly-neutralizing scFv JL427 or IgA from patients with very prolonged HIV infection. As these Abs recognize the native conformation of the 421-433 CD4BS sequence, the procedure identifies the IE-HIV species expressing the native 421-433 CD4BS conformation.

The IE-HIV species may be tested for immunogenicity in experimental animals as in Example 2, Examples 4-8 and Examples of Methods. The immunogenicity tests include measurement of the ability of induced Abs to bind the 421-433 CD4BS sequence, catalyze the hydrolysis of gp120 and neutralize genetically diverse HIV strains. In addition, the Abs may be capable of removing virus through certain Fc-dependent functions, for example, antibody-dependent cellular virus inhibition.⁷⁵ Therefore, the sera may also be tested for Fc-dependent viral removal using appropriate assays to determine the mechanism of antibody action.

### Example 4

### Improved E-Peptide Immunogen

In view of its behavior as an improved probe for neutralizing Abs to the 421-433 CD4BS epitope described in Example 1, the single epitope E-416-433 provided the opportunity to induce an Ab response focused on the 421-433 CD4BS sequence. This may potentially be a useful feature, as certain other epitopes of gp120 can induce undesirable Abs that can enhance infection.

The present invention discloses that covalent immunization with the peptide KLH-conjugated Cys-E-416-433 alone is sufficient to induce neutralizing Ab synthesis. As E-416-433 is a small and flexible peptide, the constraints placed on its conformation by the carrier protein microenvironment are critical. Although most investigators consider the carrier to be a routine component of vaccines, this does not apply to the E-416-433 immunogen. Different carriers may constrain the peptide into different conformations.⁵¹ Consequently, we took care to attach E-416-433 to KLH only via its terminus. The peptide immunogen was prepared by conjugating the N terminal Cys residue with Lys residues of KLH using the bifunctional reagent 4-maleimidobutyric acid N-hydroxysuccinimide ester. The resultant KLH-E-416-433 conjugate contained 2000 copies of E-416-433/molecule of KLH. The density of the peptides is also important for inducing an Ab response. Very low density will only induce weak, monovalent cellular signaling through the B cell receptor, whereas excessive density may increase signalizing that is too strong and results in B cell tolerance.

BALB/cJ mice and New Zealand White Rabbits were immunized with KLH-conjugated Cys-E-416-433 by the intranasal route and subcutaneous routes, respectively. Specific primary and secondary polyclonal Ab responses in serum capable of binding BSA conjugated E-416-433 were evident (FIG. 18A,B). The BSA-peptide conjugate is used for measuring binding to preclude detection of Abs to KLH. [Note that the requirement for mimicry of the native 421-433 CD4BS sequence by the immunogen and the probe used for measuring binding are not the same. Induced-fit mechanisms of binding shown in FIG. 2 indicate that an imperfect mimetic will detect neutralizing Abs that bind the native epitope, but more stringent mimicry of the native epitope is needed to induce synthesis of neutralizing Abs to the native epitope because of increased opportunity for a corrupted response directed to an irrelevant epitope conformation at the early stages of B cell differentiation]. We also observed a vaginal E-416-433 binding IgA response in the intranasally immunized mice (FIG. 18A Inset). The hyperimmune sera neutralized several strains belonging to different HIV subtypes, including coreceptor R5 and X4-strains (Table 2). The panel contained strains with widely divergent V domain sequences (up to 91% divergence for the V1-V5 domains using as reference the subtype C strain ZA009). Preimmune sera displayed only weak neutralizing activity. Affinity chromatography of serum from immunized mice on immobilized E-416-433 permitted recovery of the noncovalently bound and covalently bound Abs with enriched binding activity for BSA conjugated E-416-433 binding activity (respectively, 28-fold and 56-fold) and enriched HIV neutralizing activity (respectively, 14-fold and 71-fold; FIG. 19). This confirmed attribution of the neutralizing activity to Ab recognition of the 421-433 CD4BS sequence

In addition, FIG. 20 demonstrates the ability of the Abs produced by immunization to bind intact HIV. Binding of intact, infection subtype C 97ZA009 virions by polyclonal rabbit IgG induced by KLH-E-416-433 immunization is shown. After incubation, HIV-IgG complexes were recovered on Protein G columns and p24 in eluates was measured. Plotted are the relative light units (RLU) captured by preimmune and immune IgG. Taken together, the findings indicate that Abs induced by immunization with KLH-E-416-433 recognize the native 421-433 CDBS sequence and neutralize HIV.

The following KLH-conjugated immunogens were also tested: E-421-433, the 421-433 peptide with a C terminal phosphonate; and Es-421-433, the 421-433 peptide with phosphonates at the side chains of Lys421 and Lys432 (FIG. 3). Nasal and intraperitoneal administration of these immunogens induced low-levels of the epitope-specific Abs in serum and vaginal fluid compared to the E-416-433 immunogen. Immunizations using E-421-433 without conjugation to KLH induced even lesser Ab titers, suggesting the facilitatory role of helper T cell epitopes expressed by the carrier protein.

### Example 5

### Improved Neutralizing Monoclonal Antibodies

In the present invention, improved MAbs were prepared by screening 1125 splenocyte hybridomas from two mice immunized with KLH-E-416-433. Seven IgM MAbs and 13 IgG MAbs with specific binding activity for BSA conjugated E-416-433 were identified.

A subset of the MAbs (10 of 20 tested; 2 IgMs and 8 IgGs) neutralized the subtype C clinical HIV isolate ZA009 (IC50 0.1-16.5 μg/ml). IgM clones with neutralizing activity are: 1F4 and 2G9. IgG clones with neutralizing activity are: 4B2-F8, 4F6-G11, 4H12-F2, 5B5-F6, 5D3-C10, 7E2-H7, 9F3-A7 and 11G8-H4.

Broad HIV neutralizing activity was confirmed (see Table 3).

Specificity for 421-433 CD4BS sequence was shown by inhibition of MAb 2G9 binding to immobilized BSA conjugated BSA-E-416-433 in the presence of soluble full-length gp120, Bt-E-416-433 and sCD4 but not by control Sh416-433 with shuffled sequence or the irrelevant protein ovalbumin (FIG. 21). Peptide mutation studies conducted as in ref 2 indicated that 5 of 8 residues essential for CD4 binding are important for MAb 2G9-epitope recognition (K421, M426, W427, V430, K32; Table 1). These observations indicate specific CD4BS recognition by the MAbs. Thus, immunization with E-416-433 induces CD4BS-specific Abs that neutralize genetically diverse HIV strains.

Molecular Features of Anti-E-416-433 Monoclonal ABS.

The VH and VL domains of the following IgM clones with specific BSA-E-416-433 binding activity were sequenced: clones 1F4, 2G2, 2G9, 2C11, H10, C11 and G12. (see sequences below). Most V domains contain few or no somatic mutations. Sequence diversification due to V-D-J and V-J recombination processes is evident.

The VH and VL domains of the following IgG clones with specific BSA-E-416-433 binding activity and HIV neutralizing activity were sequenced: clones 4B2-F8 and 9F3-A7. Both Abs shared the same VL and VH germline genes (respectively, 8-21 VK8 gene and J558.51 VH1 gene). The V domains of both MAbs contained extensive somatic mutations. The VL domains of both clones were poorly mutated (replacement/silent mutation ratios, the R/S ratios, for IgG 4B2-F8 VL and IgG 9F3-A7 domain were 1/1 and 2/1, respectively). In contrast, the VH domains were more mutated. The R/S ratios for IgG 4B2-F8 VH CDRs and FRs were respectively, 8/1 and 7/5. The R/S ratios for IgG 9F3-A7 VH CDRs and FRs were respectively, 4/1 and 3/4. The cumulative R/S ratio for FR1 and FR3 for the two neutralizing MAbs was 11/5. The expected R/S computed as in ref 76 is 8/8, suggesting immunogen-driven selection of FR mutants. The frequent FR mutations are consistent with our vaccine approach of adaptively improving the innate 421-433 CD4BS epitope recognition capability. In addition to somatic mutations in the V gene, sequence diversification of the V domains due to the V-D-J and V-J recombination processes was evident

The IgM and IgG clones displayed varying levels of BSA-E-416-433, E-120 and gp120 binding activity. Moreover, the ratios of BSA-E-416/E-gp120 and BSA-E-416-33/gp120 binding activities for the different MAb clones were divergent (respectively, by 3887-fold and 219-fold for the 20 MAbs). The widely divergent binding ratios reflects the extent to which different Ab specificities can arise due to flexibility of the 421-433 CD4BS sequence, diversity of the innate CD4BS-specific repertoire, and induced fitting of the 421-433 CD4BS into the FR-based binding site (FIG. 2). The functional importance of fine 421-433 CD4BS sequence specificity of the Abs is evident from observations that only a subset of the MAbs neutralized HIV (10 out of 20) although all MAbs displayed the ability to bind the E-416-433 probe and hydrolyze full length gp120.

Amino acid sequences were deduced from the nucleotide sequence obtained by dideoxy nucleotide sequencing of PCR amplified V domain cDNA from the hybridoma cells

IgM clone G12
VL chain:
(SEQ ID NO: __)
ENVLTQSPAIMSASPGEKVTMTCSASSSVSYMHWYQQKSSTSPKLWIYDT
SKLASGVPGRFSGSGSGNSYSLTISSMEAEDVATYYCFQGSGYPYTFGGG
TKLEIK
VH chain:
(SEQ ID NO: __)
QVQLQQPGSVLVRPGASVKLSCKASGYTFTSSWMHWAKQRPGQGLEWIGE
IHPNSGNTNYNEKFKGKATLTVGTSSSTAYVDLSSLTSEDSAVYYCARPG
IGESQSFPNVFPAAAEXLKGEFCRYPSHWRPLEHAS
IgM clone C11:
VL chain:
(SEQ ID NO: __)
DIQMTQSPATLSVTPGDSVSLSCRASQSISNNLHWYQQKSHESPRLLIKY
ASQSISGIPSRFSGSGSGTDFTLSINSVETEDFGMYFCQQSNSWPLTFGA
GTKLELK
VH chain:
(SEQ ID NO: __)
VQVQLKQSGPGLVQPSQSLSITCTVSGFSLTSYGVHWVRQSPGKGLEWLG
VIWSGGSTDYNAAFISRLSISKDNSKSQVFFKMNSLQANDTAIYYCARTG
FAYWGRGTLVTVS
IgM clone H10:
VL chain:
(SEQ ID NO: __)
QIVLTQSPAIMSASLGERVTMTCTASSSVSSSYLHWYQQKPGSSPKLWIY
STSNLASGVPARFSGSGSGTSYSLTISSMEAEDAATYYCHQYHRSPRTFG
GGTKLEIK
VH chain:
(SEQ ID NO: __)
EVKLVESGGGLVQPGGSLRLSCATSGFTFTDYYMSWVRQPPGKALERLGF
IRNKANGYTTEYSASVKGRFTISRDNSQSILYLQMNTLRAEDSATYYCAR
DNQSFYYAMDYWGQGTSVTVSS
IgM clone 1F4:
VL chain:
(SEQ ID NO: __)
VLMTQTPLSLPVSLGDQASISCRSSQSLVHSNGNTYLHWYLQKPGQSPKL
LIYKVSNRFSGVPDRFSGSGSGTDFTLKISRVEAEDLGVYFCSQSTHVPY
TFGGGTKLEIK
VH chain:
(SEQ ID NO: __)
EVKLQESGPSLVQPSQSLSITCTVSGFSLTSYGVHWVRQSPGKGLEWLGV
IWRGGSTDYNAAFMSRLSITKDNSKSQVFFKMNSLQADDTAIYYCAKRYG
NYGGGAMDYWGQGTSVTVSS
IgM clone 2C11:
VL chain:
(SEQ ID NO: __)
QIVLTQSPAIMSASPGEKVTITCSASSSVSYMHWFQQKPGTSPKLWIYST
SNLASGVPARFSGSGSGTSYSLTISRMEAEDAATYYCQQRSSYPYTFGGG
TKLEIK
VH chain:
(SEQ ID NO: __)
EVQLVESGGGLVQPKGSLKLSCAASGFTFNTYAMHWVCQAPGKGLECVAR
IRSKSNNYATYYADSVKDRFTISRDDSQSMLYLQMNNLKTEDTAMYYCVR
ERAGYFDVWGAGTTVTVSS
IgM Clone 2G2:
VL chain:
(SEQ ID NO: __)
DIVITQSPSYLAASPGETITINCRASKSISKYLAWYQEKPGKTNKLLIYS
GSTLQSGIPSRFSGSGSGTDFTLTISSLEPEDFAMYYCQQHNEYPYTFGG
GTKLEIK
VH chain:
(SEQ ID NO: __)
EVQLQQSGPELVKTGASVKISCKASGYSFTGYYMHWVKQSHGKSLEWIGY
ISCYNGATSYNQKFKGKATFTVDTSSSTAYMQFNSLTSEDSAVYYCARGG
TTVVATGKYAMDYWGQGTSVTVSS
IgM clone 2G9:
VL chain:
(SEQ ID NO: __)
DIQMTQTTSSLSASLGDRVTISCRASQDISNYLNWYQQKPDGTVKLLIYY
TSRLHSGVPSRFSGSGSGTDYSLTISNLEQEDIATYFCQQGNTLPTFGGG
TKLEIKRA
VH chain:
(SEQ ID NO: __)
QVQLKQSGPGLVQPSQSLSITCTVSGFSLTSYGVHWVRQSPGKGLEWLGV
IWSGGSTDYNAAFISRLSISKDNSKSQVFFKMNSLQANDTAIYYCARNKD
YGSSYDYYAMDYWGQGTSVTVSS
IgG clone 9F3-A7:
VL chain:
(SEQ ID NO: __)
DIVMSQSPSSLAVSAGEKVTMRCKSSQSLLNSRTRKNYLAWYQQKPGQSP
KLLIYWASTRESGVPDRFTGSGSGTDFTLTISSVQAEDLAVYYCKQFYNL
WTFGGGTKLEIK
VH chain:
(SEQ ID NO: __)
QVQLQQSGAELVRPGASVKLSCKALGYTFTDYEMHWVKQTPVHGLEWIGG
IYPGSGGTAYNQKFKGKATLTADKSSSTAYMELSSLTSEDSAVYYCTKFR
FSSFAMDYWGQGTSVTVSS
IgG clone 4B2-F8:
VL chain:
(SEQ ID NO: __)
DIVMSQSPSSLAVSAGEKVTMSCKSSQSLLNSRTRKNYLAWYQQKPGQSP
KLLIYWASTRESGVPDRFTGSGSGTDFTLTINSVQAEDLAVYYCKQSYNL
WTFGGGTKLEIK
VH chain:
(SEQ ID NO: __)
QVQLQQSGAELVRPGASVKLSCMALGYTFTDYEIHWVKQTPVHGLEWIGG
FHPGSGGGAYSQKFKGKATLIADKSSSIAYMEVISLTSEDSAVYYCTRFR
YSSFAMVYWGQGTSVTVSS

Covalent and Catalytic Activity.

Covalent immunization comes with the bonus of electrophile-driven strengthening of Ab nucleophilic reactivity.^{5,13,57} Enhanced nucleophilicity improves HIV inactivation as follows (FIG. 7). First, pairing of the strong Ab nucleophile with weakly electrophilic carbonyls forms covalent complexes. Intact HIV was bound covalently by Abs induced by E-gp120¹³ and an electrophilic gp120 V3 peptide analog.⁵⁷ Unlike reversible complexes, covalent complexes did not dissociate readily, increasing the neutralization potency.⁵⁷ Second, if accessory groups enabling water attack on the covalent acyl-Ab complex are present, peptide bond cleavage occurs. MAbs to KLH-E-416-433 hydrolyzed gp120 (FIG. 22). Catalytic rates were far superior to preimmune Abs (by 10-448 fold; catalytic efficiency from rate data at increasing gp120 concentration, 1.8−4.0×10⁵ min⁻¹M⁻¹). A single catalytic Ab molecule can be reused to cleave thousands of gp120 molecules over its biological half-life in blood (1-3 weeks), improving neutralization compared to reversibly-binding Abs.^{39,58} Note that the biotinylated gp120 fragmentation pattern does not report the entire product profile because we used sparsely labeled protein as the substrate (1-2 biotin molecules/gp120 molecule), and certain product fragments do not contain the biotin label. The product profiles for non-labeled gp120 revealed by Coomassie staining after digestion the MAbs and the previously reported preimmune catalytic Abs^{9,39} were identical (see example MAb H10, lane 6, FIG. 22A). One of the peptide bonds cleaved by preimmune IgA³⁹ and IgM⁹ identified previously is the 432-433 bond on the 421-433 epitope. N-terminal sequencing of the 17 kD product in lane 6 confirmed cleavage at this bond by an anti-KLH-E-416-433 MAb. No MAb cleavage of irrelevant proteins was detected (EGFR, BSA, HIV tat). sCD4 but not the irrelevant protein ovalbumin inhibited gp120 cleavage, providing proof for CD4BS-specific catalysis (FIG. 22B). The data suggest amplification of the constitutive catalytic Ab subset directed to the CD4BS.

Taken together, the results indicate that KLH-E-416-433 immunization favors the recruitment and adaptive improvement of preimmune catalytic activity directed to the 421-433 CD4BS sequence.

### Example 6

### Non-Human Primate Studies with Electrophilic Immunogens

Induction of E-416-433 and E-gp120 Binding ABS in Monkeys.

We immunized 3 rhesus macaques of Indian origin with KLH-E-416-433 and 3 macaques with control KLH (see example in FIG. 23A). The IgG class Abs in serum displayed binding of BSA conjugated E-416-433 with robust titers between 119,000-1,500,000 after five administrations of KLH-E-416-433. The sera also displayed specific binding to E-gp120 (FIG. 23B), indicating recognition of the 421-433 CD4BS epitope conformation expressed by E-gp120. However, binding of gp120 was very poor (<0.3 A490 at 1:20 serum dilution), consistent with FIG. 5 data indicating that insertion of phosphonate groups into gp120 improves mimicry of the native 421-433 CD4BS epitope.

To assess whether improvement in Ab activity is feasible, we immunized the three KLH-E-416-433 immunized monkeys further with E-gp120. The booster E-gp120 immunization resulted in maintenance of a steady binding activity for BSA-E-416-433 and increased E-120 binding titers (FIG. 23A).

No E-416-433 or E-gp120 IgG binding titer was observed upon control KLH immunization of three monkeys. These monkeys received three subsequent E-gp120 administrations to determine whether E-gp120 alone induces a useful response. The E-gp120 binding titers in the serum IgG fraction were 26,000-87,000 (FIG. 23C).

Induction of HIV Neutralizing Abs in Monkeys.

We measured the neutralizing activity of monkeys immunized with KLH-E-416-433 alone using primary isolates subtype C, CCR5 dependent strain 97ZA009 and PHA-stimulated human PBMCs as hosts. Only low-level neutralizing activity was observed after KLH-E-416-433 immunization (see example FIG. 24). Note that the same monkey displayed high level BSA-E-416-433 binding titer. Also note that KLH-E-416-433 immunogen alone induced superior neutralizing Ab titers in mice and rabbits (Example 1). It may be concluded that the KLH-E-416-433 immunogen induced Abs with differing neutralizing potency in monkeys versus the lower species tested. Evidently, the KLH-E-416-433 immunogen induces neutralization Abs to the native conformation of 421-433 CD4BS sequence less readily in monkeys. The differing efficacy of the immunogen in various species may be due to differences in the 421-433 CD4BS sequence specificity of the pre-existing, innate Ab repertoire. Note that the germline gene sequences encoding the V domains of mice, rabbits and monkeys is homologous but not identical. Also, HIV is thought to have evolved from a simian virus analog, whereas no HIV analog capable of infecting mice or rabbits is known. We cannot exclude differences in the specificity of the FR-based binding site in the innate Ab repertoire of various species as a factor influencing the host species selectivity of HIV.

We also measured the neutralizing activity of our monkeys immunized sequentially with KLH-E-416-433 followed by E-gp120. Sera from all three sequentially-immunized monkeys neutralized the subtype C, CCR5 dependent strain 97ZA009. The serum Abs neutralized genetically diverse strains drawn from subtype A, B, C, D, and AE with co-receptor CCR5 and CCR4 dependency (Table 4). We also verified that E-gp120 alone induces neutralizing Abs (FIG. 25). Hence, the E-immunogens induce an immune response that may be effective in preventing HIV infection across the world.

Validating HIV Neutralization In Vivo.

The simian-human immunodeficiency virus (SHIV)-macaque model is a frequent test of HIV therapies/vaccines.⁷⁰ Macaques clear SHIV rapidly, but a peak of viremia is evident at 1-2 weeks. The MAb 3A5 raised by immunization with E-120 with specificity for the 421-433 CD4BS sequence,¹² neutralized SHIV_{SF162P3} strain with potency comparable to HIV (IC50˜3 μg/mL; FIG. 26A). Intravenous MAb infusion suppressed the viral load in macaques challenged with SHIV_{SF162P3} compared to the PBS-infused control group (FIG. 26B) despite its comparatively rapid clearance from blood (half-life 8.8 hrs). The data provide preliminary in vivo validation of Ab neutralizing activity in tissue culture.

### Examples of Vaccine Testing and Optimization

### Example 7

### Immunogenicity of the Vaccine in Rhesus Macaques and Protection Against SHIV Infection

In vivo proof-of-principle for protection against viral challenge in monkeys is a significant milestone in developing an HIV vaccine. SHIV strains infect rhesus monkeys. Desirable features of an effective vaccine candidate candidate are: (a) Induce Abs that neutralize diverse HIV strains in tissue culture; (b) Protect against infection contracted by vaginal SHIV administration; (c) Induce B cell memory that can be amplified by subsequent exposure to SHIV.

Protection against SHIV. E-120, KLH-E-416-433 or another electrophilic immunogen combined with a suitable adjuvant is used to induce broadly neutralizing polyclonal Ab responses. For example, one group of 8 female monkeys receives IE-gp120 in adjuvant at 2-week intervals (Experiment A, FIG. 27A). The second group of 8 monkeys receives equivalent administrations of the adjuvant alone. Examples of adjuvants for intranasal (IN) and intramuscular (IM) immunization are the R192:G heat-labile E. coli enterotoxin mutant (LTm), RIBI and alum. These adjuvants were used successfully in the foregoing Examples. Contemporaneous IN and IM immunizations are designed, respectively, to afford robust mucosal and systemic Ab responses. Immunizations are continued until the SHIV_{SF162P3} neutralization titers determined by tissue culture infection assays in the cervicovaginal lavage fluid (CVLF) and serum are at least 1:50 and 1:5, respectively. Samples from animals receiving adjuvant alone serve as negative controls. The immunogen dose and number of boosters can be increased or decreased to maximize the neutralization titer. An example dose of IE-gp120 is 30 μg/kg body weight.

Based on the infection kinetics and HIV amounts in semen, it has been suggested that neutralizing Ab titers of ˜1:40 may be sufficient to prevent transmission.^{77,78} This corresponds to an observed neutralization titer of ˜1:4 in diluted CVLF, if it is assumed that the vaginal lining fluid is diluted by 10-fold or more in the lavage procedure.

Seven days after the requisite Ab titers have been reached, the monkeys are challenged vaginally with SHIV_{SF162P3} (see Methods). This is a hybrid virus containing env, tat, rev and vpu genes of the CCR5-dependent subtype B strain HIV strain SF162 cloned into the SIV_{mac239} genome that is reliably transmitted by the mucosal route.⁷⁹ Most sexually transmitted HIV infections world-wide occur by the vaginal route. The rectal transmission route may also be tested in this model. SHIV_{SF162P3} is a comparatively ‘difficult-to-neutralize’ strain for most anti-HIV Abs.⁸° Abs to the 421-433 CD4BS sequence neutralize SHIV_{SF162P3} (FIG. 26A). Most monkeys are infected by this protocol. In this example, the study is powered to detect a meaningful difference in viremia between groups at the end of the study. With an estimate of 6 infected monkeys/group, the study is powered to detect an effect size of 1.8 with 80% power and 5% significance level by the 2-sided t-test [effect size: (Group 1 mean viremia—Group 2 mean viremia)/s.d.]. Peak viral loads are usually observed within a few weeks of SHIV challenge. Infection is monitored in blood and CVLF collected 24 h after virus challenge and at weekly intervals through week 20 (FIG. 27A). Viral RNA and DNA are measured in plasma, CVLF and PBMC aliquots. Cell-associated virus is assayed by co-culturing PBMC with an indicator cell line and determination of infected cells by staining for β-gal.⁸¹ CD4+ T cell counts in blood are also monitored. At euthanasia, the major lymphoid tissues and vagina/cervix are obtained for measurement of the viral DNA and RNA content (spleen, inguinal and axillary lymph nodes, mesenteric lymph node, intestinal lamina propria). Decreased viremia induced by IE-gp120 immunization compared to the adjuvant alone control indicates the potential of the prototype vaccine to protect against HIV infection.

SHIV/HIV Neutralization.

It is important to assess possible escape mutations and utility of our vaccine approach to diverse HIV strains found across the world. Initially, SHIV_{SF162P3} and the difficult-to-neutralize subtype C, CCR-5 dependent clinical HIV isolate ZA009 are tested for neutralization by unfractionated sera and CVLF samples from IE-gp120 immunized animals. Samples obtained before and after challenge with SHIV along with control preimmune serum and CVLF are tested. Neutralization is quantified using PBMCs from non-infected humans as described.⁴⁴ In repeat assays, purified IgG, IgA, IgM and SIgA are tested. In some assays, monocyte-derived macrophages are used as hosts. Nonspecific cytotoxicity is analyzed by Ab treatment of PBMCs and vital staining.

In addition to subtype B SHIV_{SF162P3} strain, neutralization of genetically diverse clinical HIV isolates is measured using serum and CVLF samples. At least 4-6 clinical HIV isolates from each of subtypes A, B, C, D, E (AE env recombination) with V domains that are highly divergent in sequence are studied, including coreceptor R5-, X4- and R5X4-dependent strains. Panels of virus strains with varying resistance to Abs and known 416-433 sequences have been assembled.^{2,12} Residues 421-433 are largely conserved across Group M HIV-1 strains. To judge the possibility of viral escape, attention is given to Ab neutralization of strains with sequence differences at positions without effect on gp120-CD4 binding versus positions important for the binding. Potential emergence of escape mutations is tested by coculturing PBMCs infected chronically with two virus strains (SHIV_{SF162P3}, ZA009) with the anti-IE-gp120 serum over 10 passages for 70 days. At each passage, the virus in the culture supernatants is titered using a fresh batch of PBMCs. The sequence of the gp120 gene in supernatants displaying detectable infectivity is determined to identify the amino acids residues permitting development of resistance to the Ab.

To quantify the contributions of reversible binding and catalysis in HIV neutralization, the neutralization assay (strain ZA009) are done in the presence of the E-Hapten 1 and control Hapten 2. E-Hapten 1 permits reversible binding but inhibits Ab catalysis. Hapten 2 is the control phosphonic acid devoid of inhibitory activity. Reduced neutralization in the presence of E-Hapten 1 suggests that catalysis enhances Ab neutralizing activity.

Ab neutralization of genetically diverse HIV strains and no or minimal viral escape mutants can be interpreted to suggest the feasibility of vaccination around the world, as opposed to relying on constantly changing vaccine formulations for viral subtypes or new virus strains (e.g., influenza virus vaccines are reformulated as new viral variants evolve). The CD4BS region is essential for infection. This provides a selective pressure against viral escape from anti-CD4BS Abs.

Ab Characterization.

Ab binding of IE-gp120 and BSA-conjugated E-416-433 is measured by ELISA. IgG, IgA and IgM titers will be measured separately to confirm that there is no class restriction. Increasing specific binding of BSA-E-416-433 induced over the course of KLH-E-416-433 immunization indicates that an adaptive Ab response has occurred. The specificity of immobilized BSA-E-416-433 binding by plasma and CVLF Abs is confirmed by competitive inhibition with soluble CD4 as described.² Cleavage of purified gp120 is measured using electrophoretically homogeneous secretory IgA (SIgA) from CVLF and IgA, IgG and IgM from sera after purification by affinity chromatography (see Methods). The catalysis data are confirmed by showing retention of the activity in Ab preparations prepared by denaturing gel filtration (to remove noncovalently associated trace contaminant) ^{9,82} Peptide bonds cleaved by Abs are identified from the mass distribution of gp120 fragments on electrophoresis gels and by N-terminal sequencing ⁹ Inhibition of catalysis by sCD4 but not irrelevant proteins is quantified to confirm noncovalent CD4BS recognition by the catalytic Abs. Apparent Kₘ (approximate measure of KO and apparent maximal velocity/unit Ab mass (Vₘₐₓ) are computed from rates observed at varying gp120 concentrations.⁹ In addition to purified gp120, the trimeric gp120 expressed by intact HIV particles is employed as substrate to establish binding and catalytic hydrolysis attributable to recognition of the native 421-433 CD4BS sequence (see FIG. 20 and Methods). To establish Ab specificity, we test the binding and catalytic hydrolysis of the following panel of purified human antigens biotinylated at Lys side chains: Factor VIII, myelin basic protein, thyroglobulin, soluble CD4, the extracellular domain of the epidermal growth factor receptor, transferrin, human IgG, and myosin. Binding is determined by ELISA.

Vaccine Toxicity/Ab Cross-Reactivity.

The control and IE-gp120 immunized animals are monitored as follow: (i) Evaluation of immunogen administration site for irritation/inflammation; (ii) Behavioral examination; (iii) Physical examinations (body weight, body temperature, blood pressure); (iv) Blood/urine cellular and chemistry profiles; (v) At autopsy, gross observation, organ weights and histopathology evaluation of various organs. Blood coagulation occurs by a series of serine protease reactions. To rule out nonspecific interference with blood coagulation, we monitor the activated partial thromboplastin time and prothrombin time ratio as reporters of the intrinsic and extrinsic coagulation pathway, respectively. Ab cross-reactions with human tissues is studied to assess the possibility of autoimmune damage. As an example, the following de-identified tissues obtained at autopsy from humans without HIV infection are tested: heart, lung, kidney, liver, stomach, small and large intestine, spleen and brain. Cryostat tissue sections treated with monkey anti-IE-gp120 IgG or control monkey IgG from animals immunized with adjuvant alone are stained with peroxidase-conjugated Abs to monkey IgG and examined by microscopy. Equivalent tissue staining by the anti-E-416-433 IgG and control IgG suggests lack of cross-reactions with human proteins. The Ab-treated sections are also be stained with peroxidase-conjugated Abs to commonly expressed tissue antigens such as actin, tubulin, myosin, vimetin and CD56. Unimpaired staining of these antigens suggests lack of indiscriminate polypeptide digestion by the Abs.

SHIV-Induced Protective Memory Response.

An important feature of vaccines is the induction of B cell memory enabling a subsequent protective Ab response upon contact with microbe. Therefore, SHIV challenge is also performed after the plasma and CVLF neutralizing Ab titers have declined to low levels (<10% of peak Ab response in Experiment B, FIG. 27B; measured every 4 weeks after the final IE-gp120 booster). Amplification of Ab levels in plasma and CVLF induced by challenge with SHIV measured by SHIV neutralization assays indicates SHIV stimulation of E-416-433 induced immune memory. Experimental end-points to determine whether the monkeys are protected against the infection are as before. A significant reduction of viremia indicates the ability of E-416-433 immunization to induce immune memory that can protect against infection upon subsequent contact with SHIV.

### Example 8

### Carrier Protein and Adjuvant Optimization

For peptide immunogens, the carrier protein is an important factor governing the quantity and quality of the Ab response. In addition, the adjuvant is important to maximize the Ab response both for peptide and protein immunogens. Alternate carriers and adjuvants are tested using monkeys or rabbits to optimize the vaccine formulation.

Carrier/Adjuvant effects.

Rabbits offer a well-established animal model to study candidate vaccines, affording sufficient production of mucosal and systemic Abs for detailed analysis of functional properties. In the examples shown here, New Zealand White female rabbits are used (n=4/group). Inducing mucosal immunity is important to prevent HIV transmission. Inducing systemic immunity is important for controlling spread of infection.

As an example, immunizations are done by alternate intranasal and intramuscular KLH-E-416-433 administration at 2 week intervals to induce strong mucosal as well as systemic Ab responses as described previously.⁸³ Intranasal immunization results in broad and specific B cell immunity expressed at distant mucosal sites, including the genitals and gastro-intestinal tract.⁸⁴ B cell responses to peptides depend in part on generating antigen-specific helper T cells to T_{H}-epitopes expressed on the carrier protein. Costimulatory helper T cell signaling helps drive somatic diversification of the Ab V domains and Ab class-switching over the course of B cell maturation.⁸⁵

In addition to KLH, other protein carriers that support folding of the 421-433 CD4BS epitope in a near-native conformation can be used, for example, tetanus toxoid and CD40 ligand (Table 5; 300 μg peptide equivalents/rabbit). The KLH-E-416-433 conjugate contains ˜2000 copies of E-416-433 linked via an N terminal Cys located in the peptide to Lys side chains of KLH. Similar conjugates of E-416-433 to tetanus toxoid (TT) and CD40 ligand (CD40L) are prepared. Tetanus toxin and CD40L contain, respectively, 107 and 16 Lys residues. Preparation of TT entails an aldehyde reaction with amines, but sufficient underivatized Lys residues are available for the conjugation reaction. TT is often used in conjugate vaccines involving poorly immunogenic polysaccharide antigens, and it is approved for human use (e.g., ACTHIB, a TT conjugated-polyribosylribitol phosphate for H. influenza serotype b infection). Moreover, pre-existing memory acquired by childhood tetanus vaccination may help sustain the B cell response to the 421-433 epitope. Improved B cell responses due to pre-existing anti-carrier protein memory has been observed previously.⁸⁶ As an example, the TT-E-416-433 conjugate is tested in separate rabbit groups without and with prior TT immunization to assess the facilitatory role of anti-carrier memory (one IN and one IM administration at 2 wk intervals). Inclusion of CD40L as a carrier protein is shown to improve B cell Ab synthesis by virtue of the co-stimulatory signal generated upon CD40L binding to CD40 expressed on activated T cells.⁸⁷ To minimize possible loss of CD40L functional activity, we can adjust the number of E-416-433 copies to 3-5/CD40L molecule. The mucosal and systemic adjuvants can be LTm and RIBI, as these adjuvants were verified to support the desired Ab response.

Adjuvant can enhance the Ab response by several log orders. Adjuvants improve the immune response by virtue of various physical and chemical factors, including: improved immunogen bioavailability mediated by adsorptive effects; provision of a hydrophobic environment that improves immunogen interactions with cell surface receptors; and stimulation of innate immunity pathways. Adjuvants can activate specific toll-like receptors and other receptors on antigen-presenting cells and T cells, thereby inducing release of cytokines and expression of costimulatory molecule. Adjuvants that present the immunogen within small-sized physical units generally offer improved responses.

As examples, in addition to Ribi and LTm, various systemic and mucosal adjuvants can be used (Table 5). Aluminum hydroxide (alum) is approved for human use. This adjuvant adsorbs immunogens and then releases it slowly. In addition, it was recently found to activate innate immunity via the nucleotide-binding domain leucine-rich repeat-containing protein 3 ‘inflammosome’ pathway.⁸⁸ W₈₀5EC is an oil-in-water nanoparticle emulsion composed of cetyl pyridinium chloride, soybean oil, Tween 80 and ethanol with mean droplet size <400 nm diameter. It is reported to facilitate induction of Abs to viral and bacterial proteins with titers in the 1:10⁶ range.⁸⁹ Cholera toxin A1 subunit linked to the DD Ig-binding domains of staphylococcal protein A, is a non-toxic TH1/TH2 adjuvant that enhances mucosal Ab responses to HIV and other immunogens. CpG ODN is a toll-like receptor 9 agonist that favors TH1 responses by actions on dendritic cells and B cells.⁹°

Characterization of Abs.

The desired Ab activities are tested in sera, CVLF and fecal pellet extracts collected from rabbits using well-documented procedures⁹¹⁻⁹³ before and 1 wk after each immunization. The Ab activities to be tested are essentially as described in Example 1 Activities that are tested are: (A) Potency with which the Abs neutralize genetically diverse HIV strains; (B) Binding of BSA-E-416-433 peptide; and (C) Catalytic hydrolysis of gp120.

Taken together, the studies are designed to identify the carrier protein and adjuvant supporting enhanced production of neutralizing Abs to the 421-433 CD4BS epitope.

### Example 9

### Improved Structural Variants of the 421-433 CD4BS Epitope

Our results predict a variety of ways to obtain improved immunogens expressing the 421-433 CD4BS sequence in a conformation that better resembles the native conformation of this sequence on the viral surface. Such improved immunogens are predicted to induce the synthesis of HIV neutralizing Abs at greater magnitude and with greater potency than current generation vaccine candidates.

Structure of Alternate E-Vaccine Candidates.

The prototype KLH-E-416-433 vaccine and E-120 contain the 421-433 CD4BS epitope recognized by the FRs of BCRs found in the innate Ab repertoire without prior exposure to HIV. Studies on the secreted Abs from non-infected humans have indicated that the innate BCRs recognize the native 421-433 CD4BS sequence of the virus sufficiently to neutralize HIV (FIG. 12C). Electrophilic phosphonates are located on Lys side chains to bind BCRs covalently. The covalent binding results in bypass on deficient Ab class-switching over the course of B cell differentiation, permitting adaptive synthesis of neutralizing Abs upon covalent immunization with the KLH-E-416-433 and E-gp120

As examples, five classes of immunogens described below can be tested for improved 421-433 CD4BS conformation.

For each class of test immunogens, the structure of the electrophile can also be varied to provide optimum reaction with nucleophiles expressed by the Abs on B cells. For example, phosphonate monoesters, aldehydic or keto compounds, dicarbonyl compounds, lipid peroxidation products, boronate compounds and vanadate compounds can be employed as alternate electrophiles. The chemical constitution and length of the linker can also be varied. Examples of dicarbonyl electrophiles included the Advanced Glycation Endproducts obtained by the reaction of sugars with proteins. Another example of a protein electrophile is the reaction product of 4-hydroxy-2-nonenal with protein groups such as the nucleophilic side chains of Lys residues (FIG. 2). This reaction generates an electrophilic protein that can react with Ab combining site.

As an example, in the phenylphosphonate structure with E-416-433 in FIG. 4, modulation of the electrophilic reactivity of the electrophilic phosphorus atom can be achieved by introducing various types of negatively charged and positively charged substituents close to the phosphorus atom. Examples of such substituents placed in the phenyl ring are shown in FIG. 3 (R1 and R2 groups). The result is that electron withdrawing and donating capacity of the compound can be altered, thereby optimizing its reactivity with Ab nucleophiles.

In every case, an appropriate carrier protein and adjuvant are used to prepare the candidate vaccine formulation using methods described in Example 8.

The five classes of novel immunogens are:

A. Degenerate E-416-433 (degE-416-433). The KLH-E-416-433 immunogen contains the consensus epitope sequence. Table 1 shows the extent to which individual amino acids of the epitope are conserved in 1699 Group M HIV strains available in the databanks Abs to the 421-433 CD4BS epitope neutralized all HIV strains potently, but the neutralizing potencies were superior by 2-3 log orders for across the panel of strains.² The variable potency may derive in part to epitope sequence divergences. Consequently, a degenerate degE-421-433 immunogen that includes peptides expressing greater sequence identity with the individual epitope sequences of diverse HIV strains can induce Abs with more consistent neutralizing activity across the strains. Conservation of the epitope sequence is >95% at all but 4 of its 18 positions. As examples, degeneracies at these 4 positions can be introduced with the objective of including peptide species in which the rare sequence divergences are better represented. An example degE-416-433 immunogen is composed of 24 peptides with the following sequence: L-P/Q-C-R-I-K-Q-I-I-N/R-M/R-W-Q-E/R/G-V-G-K-A. This immunogen contains E-peptide species with the amino acids at individual positions encompassing >95% of Group M HIV strains.

B. Rigidified E-416-433. Short peptides are flexible and can fold into alternate conformations.^{52,94} Induced-fit binding mechanisms can force KLH-E-416-433 bound to BCRs into a conformation deviating from the native CD4BS conformation expressed on the HIV surface (FIG. 2). This type of binding will neither recruit the rare preimmune BCRs with innate ability to recognize the native epitope conformation nor drive adaptive production of Abs to the native CD4BS conformation. The problem can be minimized by introducing structural constraints that permit immunogen folding into a ‘rigidified’ conformation mimicking the native conformation. Crystallography and mutagenesis studies have suggested that residues 425-430 play a critical role in CD4 binding.^{10,16} Secondary structure prediction with the Chou-Fasman algorithm suggested that the 5418-V430 region of the epitope can assume alternate structures with pronounced α-helix or β-sheet content.⁵³ The 425-430 region is found mostly in β-sheet state in the gp120 crystal structure. Improved CD4 binding by synthetic peptides corresponding to the 421-433 region has been reported in the presence of organic compounds that stabilize the helical conformation.⁵²

As an example, the hydrocarbon stapling' method⁹⁵ can be used to produce rigidified E-416-433 variants with stabilized α-helix or β-sheet structures, βE-416-433 and (3E-416-433. FIG. 28A is an axial helical wheel projection of the 416-433 epitope. βE-416-433 is synthesized by introducing an 8-carbon covalent linker between two α-alkenyl alanine residues that replace S418 and Q422 (the ‘staple’; red connector). These residues do not make substantial contact with CD4 and they are not critical for recognition by neutralizing Abs (Table 1). Positions 424 and 431 are also suitable for stapling because they lie on the same face of the helical wheel and are 4-residues distant from each other, approximately corresponding to one α-helix turn. Such helical turns are nucleation sites that propagate helix formation to proximal polypeptide regions. Therefore, the staple should stabilize the 425-435 CD4 binding region in a helical conformation. Precedents for this strategy are documented (e.g., helix propagation by introducing a nucleation sequence in calmodulin).⁹⁶ The cross-link between the α-methyl, α-alkenyl residues is generated by ruthenium-catalyzed olefin metathesis. FIG. 28B shows 13E-416-433 in which the staple is a 6-carbon crosslinker between α-alkenyl residues at positions 423 and the epitope C-terminus (corresponding to gp120 M434). As this crosslink does not support helix formation, the stapling procedure should stabilize the critical 425-430 CD4 binding residues in its natural conformation (potentially, the β-sheet conformation seen by crystallography). FIG. 28C shows the α-alkenyl residue structure and the resulting cross-link. Folding of the E-416-433 analogs into the desired secondary structure is determined by circular dichroism (CD) followed by spectral deconvolution to yield the α-helix and β-sheet content.⁹⁷

An additional example of an improved immunogen likely due to a rigidification effect is evident from comparison of the E-416-433 peptide with its non-electrophilic peptide 416-433 counterpart devoid of the electrophilic phosphonate groups. FIG. 29 shows the superior recognition of KLH-E-416-433 compared to KLH-NE-416-433 by soluble CD4 (sCD4) and neutralizing Abs directed to the 421-433 CD4BS sequence (IgA from patients with HIV infection for 19-21 years described in ref 2 and MAb YZ23 described in ref 12). The superior recognition of the electrophilic peptide compared to the nonelectrophilic peptide explains induction of broadly neutralizing Abs by the former immunogen described in Example 1. The phosphonate-linker group incorporated into E-416-433 is bulky and likely to help constrain the peptide sequence into a restricted and favorable conformational state.

C. CD4BS expansion. Lengthening the 416-433 epitope further can provide additional stabilization interactions in the folding of the synthetic peptide, resulting in improved mimicry of the native CD4BS. For example, we observed improved CD4 binding by E-414-439, a CD4BS mimetic containing the additional 414-415 and 434-439 residues drawn from the Group M consensus gp120 sequence (FIG. 30). As for E-416-433, the electrophilic phosphonate was located on the Lys side chains of E-414-439. E-414-439 also displayed improved binding to established neutralizing Abs directed to the CD4BS, for example scFv JL427 (FIG. 30D). Consequently, E-peptides containing additional peptide sequences on the N terminal and C terminal sides of the 416-433 region can induce Abs that recognize the native CD4BS potently. Initial evidence for the functional consequences of the improved conformational state of the 421-433 CD4BS sequence is available. FIG. 31A shows HIV neutralizing activity of polyclonal serum Abs from mice immunized with KLH-E-414-439 or KLH-NE-414-439. The neutralization data are normalized for the binding titers of the sera to the appropriate immunogen determined by ELISA (BSA-E-414-439 or BSA-NE-414-439). Improved neutralizing potency per unit immunogen binding activity is evident. This suggests that an improved neutralization due to a difference in specificity of the Abs induced by the KLH-E-414-439 and KLH-E-416-433 immunogens. A difference in the specificity of the two types of Abs was also evident from superior E-gp120 binding by the Abs from KLH-E-414-439 immunized mice, evident from the differing ratios of E-gp120/E-immunogen binding in FIG. 31B.

Further lengthening of the peptide flanks of the 421-433 CD4BS sequence can improve its conformation. However, the sequence of gp120 in group M HIV strains is increasingly variable as the flanks are lengthened. To avoid excessive structural diversity, it is advisable to limit the immunogen length to about 49 amino acids corresponding to the consensus sequence of gp120 surrounding the 421-433 CD4BS sequence (numbered residues 406-459 according to strain HXB2 numbering system employed in the present invention; note that the HXB2 strain contains a 5 residues insert not present in the consensus sequence). The consensus sequence corresponding to group M HIV-1 gp120 amino acid position 406-459 is:

### NNTITLPCRIKQIINMWQGVGQAMYAPPIEGKIRCTSNITGLLLTRDGG.

The means for expanding the epitope usefully are not limited to the contiguous gp120 regions flanking the 416-433 region. Spatially remote amino acids or peptide regions can be included on the two flanks to improve the vaccine quality. For example, the outer domain residues 368-370 are thought to be components of the CD4BS along with the 421-433 sequence. A peptide sequence that spans residues 368-370, for example synthetic peptide 365-371, may be included on the N or C terminal flank of E-416-433. A linker can be placed between residues 365-371 and residues 416-433 to approximate the distance between these regions on the surface of gp120. Such expanded CD4BS immunogens may be expected to improve the induction of neutralizing Abs to HIV.

D. E-mimotopes. In the course of studies of synthetic peptide 416-433 in which individual amino acids were replaced by an irrelevant residues (Alanine), we noticed that certain replacements result in improved peptide binding to known neutralizing Abs directed to the 421-433 CD4BS sequence. Concordant but modest improvements were evident for binding of such mutant peptides by MAb IgM 2G9 raised by immunization with KLH-E-416-433 and IgA from long-term survivors of HIV infection. As examples, replacement of Pro417, G1n422 or Glu429 resulted in improved binding to these Abs, evident from ELISA competition studies (Table 6).

From the fortuitous improvements in binding displayed by the mutant 416-433 peptides, it can be predicted that a mimotope with conformation similar to the native 421-433 CD4BS sequence can be isolated from a random peptide library provided the appropriate selection and screening technologies are applied. It is well-known that screening of large libraries composed of peptides with random sequence can yield rare mimotopes that do not have the same linear sequence of the native epitope but mimic the conformation of the native epitope. Indeed, even small molecule analogs of the native epitope can be identified by screening of small molecule libraries. Molecular modeling of the Ab-ligand interaction can be done to guide the refinement of antigen structures that mimic the native epitope optimally, for example by introducing non-polar or polar substituents with varying bulk into the antigen structure. Libraries of peptides and small molecules are available commercially, and synthetic procedures for semi-rational improvement of the mimotope are also well-established.

Methods for displaying the random peptide library on a suitable surface followed by selection and screening of the desired mimotopes are well established. For example, the library can be displayed on the surface of M13 phage or ribosomes.^{98,99} In the example of a phage displayed library, selection is done using immobilized sCD4 or an immobilized neutralizing Ab to the 421-433 CD4BS sequence (for example scFv JL427). Thereafter, individual phage peptide clones with the desired binding activity are identified by screening for CD4 or Ab binding using ELISA methods.

Implementation of these procedures to the 421-433 epitope can be predicted to yield a mimotope with a conformation mimicking the native 421-433 CD4BS sequence. One or more electrophilic group can then be incorporated into the mimotope at the side chain of an appropriate amino acid, and the resultant E-mimotope can be tested as immunogen for induction of neutralizing anti-CD4BS Abs in experimental animals.

E. Binary epitope E-immunogens. An effective HIV vaccine must induce immunological memory stimulated upon contact with the virus. If deficient class-switch is the only impediment in the natural immune response, once a class-switched memory response has been induced by covalent immunization, contact with HIV should stimulate differentiation of the memory B cells into plasma cells. However, if unforeseen post-class switch immune impediments exist, then contact with the virus may not stimulate the covalent immunogen induced memory cells sufficiently. A binary E-vaccine is predicted to induce memory B cells with specificity for a second epitope in addition to the 421-433 CD4BS epitope. As the binary E-vaccine and HIV share the second epitope, the CDRs expressed by the memory cells will bind HIV, generating a stimulatory signal that overcomes down-regulatory FR binding to the viral 421-433 CD4BS sequence (FIG. 32). As examples, a second epitope can be linked to the single epitope immunogen with the best conformation of the 421-433 CD4BS sequence (designated E-CD4BS-Li-Epitope2 immunogens). The second epitope can be composed of the consensus residues 301-311 or 322-334, corresponding to the ascending or descending V3 domain limbs. These epitopes are mostly conserved (>82% identity in subtype B). Epitope2 is attached to a Cys residue at the N terminus of E-CD4BS using a Gly/Ser linker (Li). Linker length approximates the distance between the 2 epitopes measured on the surface of the gp120 crystal structure.

Immunogen Testing.

Immunogens are tested as in Examples 1 using groups of 4 rabbits each. As examples, the following immunogens are tested: the binary E-CD4BS-Li-Epitope2 immunogens, degenerate degE-416-433, the stapled E-immunogens, the lengthened E-immunogens and the E-mimotope immunogens. The immunogens are coupled to KLH as before and rabbits receive alternate intranasal and intramuscular immunogen administrations in LTm and Ribi, respectively. Blood, CVLF, feces and lymphoid tissues are collected for Ab studies.

The following tests are done to determine the quantity and quality of the Abs induced by the immunogens: (A) Potency of neutralization of genetically diverse HIV strains: (B) Binding to the BSA-conjugated immunogens; and (C) Catalytic hydrolysis of gp120.

Emergence of viral escape mutants is tested by coculturing infected cells with an Ab preparation. The degE-421-433 immunogen more comprehensively represents the diversity of Group M HIV epitope sequences. Abs to this immunogen may display improved breadth of neutralization across diverse HIV strains, determined by testing the panel of genetically diverse strains from various HIV subtypes.

Memory tests are conducted to compare HIV stimulation of B cell memory induced by the single epitope E-CD4BS immunogen and the binary E-CD4BS-Li-Epitope2 immunogens administered to groups of 6 rabbits each. The E-CD4BS binding titers are measured every 4 weeks after the final immunogen administration until the titer decreases to low levels. Then, a subgroup of 3 rabbits from each group receive a single booster of the appropriate E-CD4BS immunogen (positive control). The second subgroup of 3 rabbits from each group receive a booster of photochemically-inactivated intact HIV (strain ZA009). Another control group of rabbits without prior E-immunogen exposure receives only the HIV administration. One week later, the HIV neutralizing activity of Abs in the blood, CVLF and fecal extracts is measured. Rabbits that receive HIV alone are anticipated to display only low-level Abs directed to the 421-433 CD4BS epitope. Productive HIV stimulation of the E-immunogen induced memory is suggested by increased neutralizing Ab synthesis following the HIV booster.

Specific MAbs are generated to evaluate the functional consistency of individual Abs constituting polyclonal Ab preparations. Splenocytes are from a rabbit immunized with the immunogen affording the greatest neutralizing polyclonal Ab response. The rabbit myeloma 240-W derived cell line is the fusion partner.^{100,101} Hybridomas are screened for HIV neutralizing activity and cloned by limiting dilution. MAbs are purified by affinity chromatography (Protein G or anti-Ig columns).

The rigidified E-immunogens can induce Abs with superior neutralizing potency due to stable mimicry of the native epitope. The degenerate E-immunogen can induce Abs with more consistent neutralizing activity across diverse HIV strains found worldwide. Combined immunizations using the single epitope E-immunogen and the binary E-immunogens may help induce B cell memory that is more readily stimulated upon contact with HIV itself. The catalytic activity of the Abs is anticipated to improve neutralizing potency.

Our singular focus on the 421-433 epitope is open to the criticism that alternate epitopes may be more suitable vaccine targets. This is not the case. HIV offers very few conserved epitopes suitable for vaccine targeting. Indeed, no immunogen other than the E-immunogens are known to induce a neutralizing Ab response to the CD4BS. The superantigenic character of the CD4BS is a newly recognized challenge in HIV vaccine research. Our covalent immunization strategy is a viable solution to this challenge.

### Example 10

### Prophylactic and Therapeutic Utility

Prophylaxis

Controlling the HIV pandemic globally requires a prophylactic HIV vaccine. The E-immunogens disclosed in the present invention may be developed to prepare an effective prophylactic vaccine that is globally effective by virtue of inducing synthesis of Abs to the CD4BS that neutralize diverse Group M HIV strains found worldwide. Vaccination with an E-immunogen early in childhood can be foreseen as a way to prevent HIV infection. Adult vaccinations are also feasible. To maintain immune memory, periodic booster administrations of the E-immunogen will likely be necessary. Inducing mucosal immunity, for example by intranasal or oral immunization, is important to generate secretory IgA responses that reduce the probability of mucosal transmission of HIV. Inducing systemic immunity is important to minimize spread of infection by HIV that may breach the mucosal barrier.

Gene immunoprophylaxis using an Ab to the CD4BS that is produced systemically and locally in the vagina or rectum by means of a suitable vector, for example an adeno-associated viral vector (AAV), may also be feasible. AAV vectors providing Ab fragments over long durations of months to years have been developed. The procedure entails, for example, expression of neutralizing Ab variable domain genes cloned in a non-toxic, minimally immunogenic AAV vector in epithelial cells or muscle cells. This permits secretion of the Ab variable domains into mucosal fluid and/or blood. A suitable molecular form of the variable domains is the single chain Fv containing the VL and VH domains linked by a flexible peptide.

The foregoing immunoprophylactic strategies are anticipated to be effective at low cost and with minimal side effects. A variety of small molecule drugs that target the HIV reverse transcriptase, protease and integrase have been developed. Use of such drugs alone or as combined regimens by the sexual partners of infected humans has been proposed as a way to prevent HIV infection. However, these drugs are comparatively expensive and their routine use can induce serious side effects.

The side effects of the available small molecule drugs include diarrhea, nausea, vomiting, lipodystrophy, hyperglycemia, liver toxicity, pancreatitis, neuropathy, adverse nervous system effects (depression, suicidal ideation and paranoia) and increased rate of certain infections. Patient compliance is a problem due to the side effects.

Therapy

Combinations of the small molecules drugs are the mainstay of HIV therapy (highly effective anti-retroviral therapy, HAART). HAART reduces viral load by several orders of magnitude, sometime to undetectable levels. However, in addition to the problems of side effects noted above, about 10-20% of patients develop drug-resistant HIV strains.

Therapeutic vaccination with E-immunogens is a viable strategy to control infection if the vaccine induces sufficient production of broadly neutralizing Abs that do not permit development of viral escape mutants (Ab-resistant strains). Therapeutic vaccination can be initiated early after diagnosis of infection when the immune system is fully competent in mounting the vaccine-induced Ab response. Overall immune exhaustion in infected patients occurs only at the very advanced stage of infection, and the immune system generally remains competent over several years prior to progress of the infection to AIDS. Thus, finding a window of time sufficient to initiate therapeutic vaccination is not a problem.

Immunotherapy of HIV infection using intravenously infused catalytic Abs disclosed in the present invention is feasible. Previous therapy trials of anti-HIV Abs have not been successful because of insufficient neutralizing potency, insufficient ability to neutralize genetically diverse HIV strains and emergence of Ab-resistant strains. Targeting of the 421-433 CD4BS sequence by catalytic Abs is anticipated to minimize the problems encountered with other types of Abs.

In addition to intravenous infusion, the catalytic Abs disclosed in the present invention could be delivered by means of the AAV expression vector described above. The gene immunotherapy approach will reduce the costs of treatment.

The foregoing therapeutic vaccination and catalytic Ab approaches could be combined with HAART to maximize efficacy and reduce the requirement for toxic HAART regimens.

The HIV genome integrates into host chromosomes, giving rise to the problem of viral latency. Drugs that may address this problem are under study, for example drugs that induce virus packaging from the latent viral genomes by activating certain cell surface receptors. The therapeutic vaccination and catalytic Ab approaches could be combined with such drugs to address the problem of HIV latency.

### Examples of Methods

Immunogens/Adjuvants.

Synthesis of peptides, E-peptides and E-gp120 has been described.^{2,3,6,57} E-414-439 and 416-433 peptides contain the consensus subtype B epitope sequence. They are constructed by solid phase synthesis with site-directed acylation at Lys side chains using the N-hydroxysuccinimide ester of the diphenylphosphonate substituent. For binary epitope E-peptide synthesis, epitope 2 is attached to a Cys residue at the N terminus of the single epitope E-vaccine using a Gly/Ser linker with an N-terminal γ-maleimidobutyryl group. Linker length approximates the distance between the two epitopes measured on the surface of the gp120 crystal structure (PDB 2B4C) using Accelrys DS vizualizer 2.0. Epitope 2 is composed of the indicated gp120 residues. For synthesis of rigidified E-416-433 analogs, the following protected linear peptides containing two α-alkenyl alanine residues are prepared by the solid-phase method: αE-416-433 precursor containing R-2-(4′-pentenyl)alanine residues at positions 418/422, and βE-416-433 precursor containing R-2-(2′-ppropenyl)alanine and R-2-(4′-pentenyl)alanine at positions 423/C-terminus. The standard 9-fluorenylmethoxy protection scheme is used except that Lys41 1 and Lys432 are protected with the 4-methyltrityl group. The protected peptide resin is treated with bis(tricyclohexylphosphine)benzylidine ruthenium (IV) dichloride under anaerobic conditions. Introduction of phosphonate groups to metathesized peptide precursors and removal of protecting groups is done in the same manner as E-416-433. Degenerate degE-416-433 peptides are synthesized by the “portioning-mixing” method. The peptide-resin is divided into the number of residues to be coupled, and coupling conducted individually. After complete acylation is confirmed (ninhydrin test), the peptide resin is pooled for additional synthesis cycles. All compounds are HPLC purified and their structures verified by mass spectroscopy (MS). The presence of all 24 expected components in the degenerate E-416-433 is verified by MS. Peptides are conjugated to Lys residues of KLH, BSA CD40L or tetanus toxoid by means of a Cys residue using a heterobifunctional reagent.⁴⁴ The conjugation reaction is measured from consumption of—SH groups. Recombinant full length gp120 is from Immunodiagnostics, Inc or Protein Scineces, Inc. Proteins are biotinylated at Lys residues.⁶W₈₀5EC is prepared as described.⁸⁹ LTm is provided by Dr Clements.¹⁴ CpG ODN2007 (TCGTCGTTGTCGTTTTGTCGTT) is prepared by routine synthesis.

Monkey Procedures.

Cycling female Indian rhesus macaques (Macaca mulatta; 4.8-6.1 kg; negative for type D retrovirus, SIV and simian T-lymphotropic virus) are studied. Intranasal immunizations is done by immunogen instillation into each nare (0.05 ml) with LTm (0.25 mg) as adjuvant. Intramuscular immunizations is done in the upper thigh using RIBI as adjuvant. Thirty days before SHIV challenge, 30 mg Depo-Provera is administered to allow more efficient infection.¹⁰² To eliminate vaginosis as a factor in the infection process, culture swabs are obtained from all animals at least 3 weeks prior to virus challenge for bacterial identification and antibiotic sensitivity testing. Animals are treated with an oral antibiotic (Enrofloxacin, 5 mg/kg daily) for 7 days. Newly expanded SHIV_{162P3} stock prepared by in vivo passage is used as the challenge virus. Typically, the stock has 30 ng/ml of SIV p2′7, TCID50 value of 5200/ml in rhesus PBMC and in vivo MID50 of 1:69.6 for vaginal transmission in Depo Provera-treated rhesus macaques. Virus challenge is done by instilling SHIV_{SF162P3} into the vagina. Blood is collected periodically from the femoral artery. CVLF is collected by recovery of 3 ml PBS instilled into the vagina. The animal is anesthetized using ketamine-HCl (10 mg/kg)/Domitor (0.03 mg/kg). Plasma viral RNA load is quantified by a real-time nucleic-acid-sequence-based amplification assay (NASBA). Viral RNA/proviral DNA load in PBMC, spleen, inguinal and axillary lymph nodes, mesenteric lymph node and intestinal lamina propria after euthanasia is quantified by NASBA and real-time PCR.^{103,104} CD4+ T-cell counts in blood is assayed by flow cytometry.^{104,105} Immunogen administration sites are evaluated pre-inoculation and daily for 3 days post-inoculation for erythema, ulceration and edema. Behavioral examinations consist of monitoring posture, mobility and food/water consumption. Physical examinations are performed weekly. Blood samples are subjected to routine CBC (includes WBC differential), hematocrit; serum chemistry with SMAC (complete chemistry/electrolytes), creatinine, alkaline phosphatase and aspartic serum transferase. Complete urine analyses are performed. Autopsy includes gross observation, organ weights and histopathological examination of heart, lung, kidney, liver, stomach and intestine.

Rabbit Immunization/Sample Collection.

New Zealand White female rabbits (n=4 per group) are immunized 4 times every 2-weeks. Intranasal immunizations consists of immunogen instillation into each nostril (0.05 ml) using LTm (0.25 mg), W₈₀5EC (final 20% (v/v)),⁸⁹ CTA1-DD (0.25 mg)¹⁰⁶ or CpG (20 μg)¹⁰⁷ as adjuvant. Intramuscular immunizations are done using RIBI (1:1; v/v), aluminum hydroxide (2 mg) ¹⁰⁸ or W₈₀5EC (final 20% (v/v)) ⁸⁹ as adjuvant. Blood is collected from the ear. CVLF is collected following instillation of PBS (1 ml) into the vagina. Fecal pellets collected over one day are homogenized in PBS (0.1 g/ml) and the supernatant containing Abs are recovered by centrifugation.

Immunochemical Ab Assays.

Secreted IgM, IgA and IgG from serum, CVLF and fecal extracts are purified to electrophoretic homogeneity using columns of immobilized anti-rabbit IgM/IgA, anti-monkey IgM/IgA or Protein G. Rabbit MAbs are prepared using the myeloma 240-W derived cell line as fusion partner. Endotoxin levels in Abs are estimated by the Limulus amebocyte lysate test using the Endosafe-PTS instrument and FDA-approved cartridges. Trace amounts of endotoxin were removed in our previous studies using anion exchange cartridges to ensure that endotoxin does not interfere in the neutralization assays.^{2,12} Ab binding activity is tested by ELISA using immobilized antigens.^{6,44} Soluble competitor peptides are used to show specificity. Photochemical psoralen inactivation of HIV has been described.¹⁰⁹ To measure binding to HIV, immune complexes of the virus are trapped on anti-Ig affinity chromatography columns and the bound fraction eluted with pH 2.7 buffer is treated with Triton X-100 to lyse HIV particles, followed by p24 ELISA.¹³ Catalytic activity is tested using purified Abs and biotinylated gp120 or irrelevant proteins by an electrophoresis assay.⁶ Appearance of small mass products and depletion of the intact gp120 band indicate cleavage. We also use a recently-standardized assay to measure cleavage of viral gp120 using as substrate ³⁵S-Met labeled HIV (˜10⁵ cpm; obtained at the void volume of a Sephacryl-100 gel filtration column). After incubation with Abs, HIV is lysed and the intact gp120/immunoreactive fragments immunoprecipated with pooled anti-HIV Abs from HIV-infected humans are analyzed by SDS-gel electrophoresis and densitometry. Kinetic parameters (Kₘ, Vₘₐₓ) are computed from rate data at increasing substrate concentration by fitting to the Michaelis-Menten-Henri equation V=Vmax*[S]/(Km+[S]).⁶ Scissile bonds are identified by N-terminal sequencing of gp120 fragments separated by electrophoresis and blotted on PVDF membranes.⁹ Viral gp120 scissile bonds are determined by a sensitive radiosequencing method using HIV labeled in tissue culture with a mixture of radioactive amino acids. As the gp120 sequence is known, the cleavage site can be deduced from the cycle in which the radiolabeled PTH-derivitized amino acids elute.¹¹° A human tissue bank is available for Ab cross-reaction studies. Tissue cryostat sections treated with the test Ab are stained with peroxidase-conjugated Ab to macaque IgG. Controls include the second Ab alone. Fc-receptor block reagent (Pharmingen) is used to eliminate binding to Fc receptors. Ab binding is quantified by computer-assisted microscopy expressed as pixels/unit area of the section.

Neutralization Assays.

Neutralization of clinical HIV isolates obtained from the NIH AIDS Reagent Repository is tested using phytohemagglutinin-activated human PBMCs pooled from 4-12 donors.⁴⁴ The virus is incubated with the test Ab sample in quadruplicate to ensure reliability. The reaction mixtures are added to PBMCs in 96-well plates. Infection is monitored using p24 enzyme-immunoassay kits. Additional confirmatory assays are done using monocyte-derived macrophages as hosts cells.¹¹¹ Neutralization of pseudovirions expressing various env genes is also measured by a luciferase assay using the TZM-b1 host cell line.⁶¹ Emergence of escape mutant in vitro is tested as follows. The stock HIV strain ZA009 is used to infect PBMC in the presence of the Ab. Infection proceeds overnight and cells are washed and resuspended in RPMI media containing the Ab. After 7 days the cell-free supernatant is harvested and used to infect a new aliquot of stimulated PBMCs (second passage). The virus is passaged 10 times with a gradual (2-fold) increase in Ab concentration with each passage. A virus aliquot is saved at each passage for sequencing.¹¹² Viral supernatants are titered at each passage using a fresh batch of PBMCs. The gp120 gene will sequenced following RT-PCR as described.¹¹³

### BIBLIOGRAPHY

1. Paul, S., Tramontano, A., Gololobov, G., Zhou, Y. X., Taguchi, H., Karle, S., Nishiyama, Y., Planque, S., and George, S. (2001) Phosphonate ester probes for proteolytic antibodies. J Biol Chem 276, 28314-28320
2. Planque, S., Salas, M., Mitsuda, Y., Sienczyk, M., Escobar, M. A., Mooney, J. P., Morris, M. K., Nishiyama, Y., Ghosh, D., Kumar, A., Gao, F., Hanson, C. V., and Paul, S. (2010) Neutralization of genetically diverse HIV-1 strains by IgA antibodies to the 120-CD4-binding site from long-term survivors of HIV infection. Aids 24, 875-884
3. Nishiyama, Y., Bhatia, G., Bangale, Y., Planque, S., Mitsuda, Y., Taguchi, H., Karle, S., and Paul, S. (2004) Toward selective covalent inactivation of pathogenic antibodies: a phosphate diester analog of vasoactive intestinal peptide that inactivates catalytic autoantibodies. J Biol Chem 279, 7877-7883
4. Taguchi, H., Burr, G., Karle, S., Planque, S., Zhou, Y. X., Paul, S., and Nishiyama, Y. (2002) A mechanism-based probe for gp120-Hydrolyzing antibodies. Bioorg Med Chem Lett 12, 3167-3170
5. Planque, S., Taguchi, H., Burr, G., Bhatia, G., Karle, S., Zhou, Y. X., Nishiyama, Y., and Paul, S. (2003) Broadly distributed chemical reactivity of natural antibodies expressed in coordination with specific antigen binding activity. J Biol Chem 278, 20436-20443
6. Paul, S., Planque, S., Zhou, Y. X., Taguchi, H., Bhatia, G., Karle, S., Hanson, C., and Nishiyama, Y. (2003) Specific HIV gp120-cleaving antibodies induced by covalently reactive analog of gp120. J Biol Chem 278, 20429-20435
7. Sapparapu, G., Planque, S. A., Nishiyama, Y., Foung, S. K., and Paul, S. (2009) Antigen-specific proteolysis by hybrid antibodies containing promiscuous proteolytic light chains paired with an antigen-binding heavy chain. J Biol Chem 284, 24622-24633
8. Paul, S., Nishiyama, Y., Planque, S., Karle, S., Taguchi, H., Hanson, C., and Weksler, M. E. (2005) Antibodies as defensive enzymes. Springer Semin Immunopathol 26, 485-503
9. Paul, S., Karle, S., Planque, S., Taguchi, H., Salas, M., Nishiyama, Y., Handy, B., Hunter, R., Edmundson, A., and Hanson, C. (2004) Naturally occurring proteolytic antibodies: selective immunoglobulin M-catalyzed hydrolysis of HIV gp120. J Biol Chem 279, 39611-39619
10. Olshevsky, U., Helseth, E., Furman, C., Li, J., Haseltine, W., and Sodroski, J. (1990)
Identification of individual human immunodeficiency virus type 1 gp120 amino acids important for CD4 receptor binding. J Virol 64, 5701-5707
11. Pantophlet, R., Ollmann Saphire, E., Poignard, P., Parren, P. W., Wilson, I. A., and Burton, D. R. (2003) Fine mapping of the interaction of neutralizing and normeutralizing monoclonal antibodies with the CD4 binding site of human immunodeficiency virus type 1 gp120. J Virol 77, 642-658
12. Nishiyama, Y., Planque, S., Mitsuda, Y., Nitti, G., Taguchi, H., Jin, L., Symersky, J., Boivin, S., Sienczyk, M., Salas, M., Hanson, C. V., and Paul, S. (2009) Toward effective HIV vaccination: induction of binary epitope reactive antibodies with broad HIV neutralizing activity. J Biol Chem 284, 30627-30642
13. Nishiyama, Y., Karle, S., Mitsuda, Y., Taguchi, H., Planque, S., Salas, M., Hanson, C., and Paul, S. (2006) Towards irreversible HIV inactivation: stable gp120 binding by nucleophilic antibodies. J Mol Recognit 19, 423-431
14. Dickinson, B. L., and Clements, J. D. (1995) Dissociation of Escherichia coli heat-labile enterotoxin adjuvanticity from ADP-ribosyltransferase activity. Infect Immun 63, 1617-1623
15. Paul, S., Planque, S., Nishiyama, Y., Escobar, M., and Hanson, C. (2010) Back to the future: covalent epitope-based HIV vaccine development. Expert Rev Vaccines 9, 1027-1043
16. Kwong, P. D., Wyatt, R., Robinson, J., Sweet, R. W., Sodroski, J., and Hendrickson, W. A. (1998) Structure of an HIV gp120 envelope glycoprotein in complex with the CD4 receptor and a neutralizing human antibody. Nature 393, 648-659
17. Mascola, J. R., and Montefiori, D. C. (2010) The role of antibodies in HIV vaccines. Annu Rev Immunol 28, 413-444
18. Xiao, X., Chen, W., Feng, Y., Zhu, Z., Prabakaran, P., Wang, Y., Zhang, M. Y., Longo, N. S., and Dimitrov, D. S. (2009) Germline-like predecessors of broadly neutralizing antibodies lack measurable binding to HIV-1 envelope glycoproteins: implications for evasion of immune responses and design of vaccine immunogens. Biochem Biophys Res Commun 390, 404-409
19. International patent application PCT/US2009/060303.,
20. Kwong, P. D., Doyle, M. L., Casper, D. J., Cicala, C., Leavitt, S. A., Majeed, S., Steenbeke, T. D., Venturi, M., Chaiken, I., Fung, M., Katinger, H., Parren, P. W., Robinson, J., Van Ryk, D., Wang, L., Burton, D. R., Freire, E., Wyatt, R., Sodroski, J., Hendrickson, W. A., and Arthos, J. (2002) HIV-1 evades antibody-mediated neutralization through conformational masking of receptor-binding sites. Nature 420, 678-682
21. Herrera, C., Spenlehauer, C., Fung, M. S., Burton, D. R., Beddows, S., and Moore, J. P. (2003) Normeutralizing antibodies to the CD4-binding site on the gp120 subunit of human immunodeficiency virus type 1 do not interfere with the activity of a neutralizing antibody against the same site. J Virol 77, 1084-1091
22. Binley, J. M., Wrin, T., Korber, B., Zwick, M. B., Wang, M., Chappey, C., Stiegler, G., Kunert, R., Zolla-Pazner, S., Katinger, H., Petropoulos, C. J., and Burton, D. R. (2004) Comprehensive cross-clade neutralization analysis of a panel of anti-human immunodeficiency virus type 1 monoclonal antibodies. J Virol 78, 13232-13252
23. Zwick, M. B., Bonnycastle, L. L., Menendez, A., Irving, M. B., Barbas, C. F., 3rd, Parren, P. W., Burton, D. R., and Scott, J. K. (2001) Identification and characterization of a peptide that specifically binds the human, broadly neutralizing anti-human immunodeficiency virus type 1 antibody b12. J Virol 75, 6692-6699
24. Beddows, S., Franti, M., Dey, A. K., Kirschner, M., Iyer, S. P., Fisch, D. C., Ketas, T., Yuste, E., Desrosiers, R. C., Klasse, P. J., Maddon, P. J., Olson, W. C., and Moore, J. P. (2007) A comparative immunogenicity study in rabbits of disulfide-stabilized, proteolytically cleaved, soluble trimeric human immunodeficiency virus type 1 gp140, trimeric cleavage-defective gp140 and monomeric gp120. Virology 360, 329-340
25. Chen, B., Vogan, E. M., Gong, H., Skehel, J. J., Wiley, D. C., and Harrison, S. C. (2005) Structure of an unliganded simian immunodeficiency virus gp120 core. Nature 433, 834-841
26. Kwong, P. D., Wyatt, R., Majeed, S., Robinson, J., Sweet, R. W., Sodroski, J., and Hendrickson, W. A. (2000) Structures of HIV-1 gp120 envelope glycoproteins from laboratory-adapted and primary isolates. Structure 8, 1329-1339
27. Huang, C. C., Tang, M., Zhang, M. Y., Majeed, S., Montabana, E., Stanfield, R. L., Dimitrov, D. S., Korber, B., Sodroski, J., Wilson, I. A., Wyatt, R., and Kwong, P. D. (2005) Structure of a V3-containing HIV-1 gp120 core. Science 310, 1025-1028
28. Mariuzza, R. A., Phillips, S. E., and Poljak, R. J. (1987) The structural basis of antigen-antibody recognition. Annu Rev Biophys Biophys Chem 16, 139-159
29. Davies, D. R., Padlan, E. A., and Sheriff, S. (1990) Antibody-antigen complexes. Annu Rev Biochem 59, 439-473
30. Wilson, I. A., Stanfield, R. L., Rini, J. M., Arevalo, J. H., Schulze-Gahmen, U., Fremont, D. H., and Stura, E. A. (1991) Structural aspects of antibodies and antibody-antigen complexes. Ciba Found Symp 159, 13-28; discussion 28-39
31. Goodglick, L., Zevit, N., Neshat, M. S., and Braun, J. (1995) Mapping the Ig superantigen-binding site of HIV-1 gp120. J Immunol 155, 5151-5159
32. Karray, S., and Zouali, M. (1997) Identification of the B cell superantigen-binding site of HIV-1 gp120. Proc Natl Acad Sci USA 94, 1356-1360
33. Graille, M., Stura, E. A., Corper, A. L., Sutton, B. J., Taussig, M. J., Charbonnier, J. B., and Silverman, G. J. (2000) Crystal structure of a Staphylococcus aureus protein A domain complexed with the Fab fragment of a human IgM antibody: structural basis for recognition of B-cell receptors and superantigen activity. Proc Natl Acad Sci USA 97, 5399-5404
34. Berberian, L., Goodglick, L., Kipps, T. J., and Braun, J. (1993) Immunoglobulin VH3 gene products: natural ligands for HIV gp120. Science 261, 1588-1591
35. Karray, S., Juompan, L., Maroun, R. C., Isenberg, D., Silverman, G. J., and Zouali, M. (1998) Structural basis of the gp120 superantigen-binding site on human immunoglobulins. J Immunol 161, 6681-6688
36. Neshat, M. N., Goodglick, L., Lim, K., and Braun, J. (2000) Mapping the B cell superantigen binding site for HIV-1 gp120 on a V(H)3 Ig. Int Immunol 12, 305-312
37. Nishiyama, Y., Karle, S., Planque, S., Taguchi, H., and Paul, S. (2007) Antibodies to the superantigenic site of HIV-1 gp120: hydrolytic and binding activities of the light chain subunit. Mol Immunol 44, 2707-2718
38. Townsley-Fuchs, J., Kam, L., Fairhurst, R., Gange, S. J., Goodglick, L., Giorgi, J. V., Sidell, N., Detels, R., and Braun, J. (1996) Human immunodeficiency virus-1 (HIV-1) gp120 superantigen-binding serum antibodies. A host factor in homosexual HIV-1 transmission. J Clin Invest 98, 1794-1801
39. Planque, S., Mitsuda, Y., Taguchi, H., Salas, M., Morris, M. K., Nishiyama, Y., Kyle, R., Okhuysen, P., Escobar, M., Hunter, R., Sheppard, H. W., Hanson, C., and Paul, S. (2007) Characterization of gp120 Hydrolysis by IgA Antibodies from Humans without HIV Infection. AIDS Res Hum Retroviruses 23, 1541-1554
40. Goodyear, C. S., and Silverman, G. J. (2003) Death by a B cell superantigen: In vivo VH-targeted apoptotic supraclonal B cell deletion by a Staphylococcal Toxin. J Exp Med 197, 1125-1139
41. Sun, N.C., Ho, D. D., Sun, C. R., Liou, R. S., Gordon, W., Fung, M. S., Li, X. L., Ting, R. C., Lee, T. H., Chang, N. T., and Chang, T. W. (1989) Generation and characterization of monoclonal antibodies to the putative CD4-binding domain of human immunodeficiency virus type 1 gp120. J Virol 63, 3579-3585
42. Kelker, H. C., Itri, V. R., and Valentine, F. T. (2010) A strategy for eliciting antibodies against cryptic, conserved, conformationally dependent epitopes of HIV envelope glycoprotein. PLoS One 5, e8555
43. Bermas, B. L., Petri, M., Berzofsky, J. A., Waisman, A., Shearer, G. M., and Mozes, E. (1994) Binding of glycoprotein 120 and peptides from the HIV-1 envelope by autoantibodies in mice with experimentally induced systemic lupus erythematosus and in patients with the disease. AIDS Res Hum Retroviruses 10, 1071-1077
44. Karle, S., Planque, S., Nishiyama, Y., Taguchi, H., Zhou, Y. X., Salas, M., Lake, D., Thiagarajan, P., Arnett, F., Hanson, C. V., and Paul, S. (2004) Cross-clade HIV-1 neutralization by an antibody fragment from a lupus phage display library. Aids 18, 329-331
45. Planque, S., Sienczyk, M., Nishiyama, Y., Salas, M., Sapparapu, G., Mitsuda, Y., Taguchi, H., Boivin, S., Nitti, G., Montefiori, D.C., Hanson, C. V., and Paul, S. Prospective clinical utility and evolutionary implication of broadly neutralizing antibody fragments to HIV gp120 superantigenic epitope. AIDS 2008-XVII International AIDS Conference, México City, México; 3-8 Aug. 2008. Abstract Number: LBPE1134
46. Planque, S., Bangale, Y., Song, X. T., Karle, S., Taguchi, H., Poindexter, B., Bick, R., Edmundson, A., Nishiyama, Y., and Paul, S. (2004) Ontogeny of proteolytic immunity: IgM serine proteases. J Biol Chem 279, 14024-14032
47. Gao, Q. S., Sun, M., Rees, A. R., and Paul, S. (1995) Site-directed mutagenesis of proteolytic antibody light chain. J Mol Biol 253, 658-664
48. Ramsland, P. A., Terzyan, S. S., Cloud, G., Bourne, C. R., Farrugia, W., Tribbick, G., Geysen, H. M., Moomaw, C. R., Slaughter, C. A., and Edmundson, A. B. (2006) Crystal structure of a glycosylated Fab from an IgM cryoglobulin with properties of a natural proteolytic antibody. Biochem J395, 473-481
49. Lacroix-Desmazes, S., Wootla, B., Dasgupta, S., Delignat, S., Bayry, J., Reinbolt, J., Hoebeke, J., Saenko, E., Kazatchkine, M. D., Friboulet, A., Christophe, O., Nagaraja, V., and Kaveri, S. V. (2006) Catalytic IgG from patients with hemophilia A inactivate therapeutic factor VIII. J Immunol 177, 1355-1363
50. Goodyear, C. S., Sugiyama, F., and Silverman, G. J. (2006) Temporal and dose-dependent relationships between in vivo B cell receptor-targeted proliferation and deletion-induced by a microbial B cell toxin. J Immunol 176, 2262-2271
51. Karle, S., Nishiyama, Y., Taguchi, H., Zhou, Y. X., Luo, J., Planque, S., Hanson, C., and Paul, S. (2003) Carrier-dependent specificity of antibodies to a conserved peptide determinant of gp120. Vaccine 21, 1213-1218
52. Reed, J., and Kinzel, V. (1991) A conformational switch is associated with receptor affinity in peptides derived from the CD4-binding domain of gp120 from HIV I. Biochemistry 30, 4521-4528
53. Gehenn, K., Pipkorn, R., and Reed, J. (2004) Successful design and synthesis of a polarity-triggered beta->alpha conformational switch using the side chain interaction index (SCII) as a measure of local structural stability. Biochemistry 43, 607-612
54. Palker, T. J., Clark, M. E., Langlois, A. J., Matthews, T. J., Weinhold, K. J., Randall, R. R., Bolognesi, D. P., and Haynes, B. F. (1988) Type-specific neutralization of the human immunodeficiency virus with antibodies to env-encoded synthetic peptides. Proc Natl Acad Sci USA 85, 1932-1936
55. Neurath, A. R., Strick, N., and Lee, E. S. (1990) B cell epitope mapping of human immunodeficiency virus envelope glycoproteins with long (19- to 36-residue) synthetic peptides. J Gen Virol 71 (Pt 1), 85-95
56. Morrow, W. J., Williams, W. M., Whalley, A. S., Ryskamp, T., Newman, R., Kang, C. Y., Chamat, S., Kohler, H., and Kieber-Emmons, T. (1992) Synthetic peptides from a conserved region of gp120 induce broadly reactive anti-HIV responses. Immunology 75, 557-564
57. Nishiyama, Y., Mitsuda, Y., Taguchi, H., Planque, S., Salas, M., Hanson, C. V., and Paul, S. (2007) Towards covalent vaccination: improved polyclonal HIV neutralizing antibody response induced by an electrophilic gp120 V3 peptide analog. J Biol Chem 282, 31250-31256
58. Berisha, H. I., Bratut, M., Bangale, Y., Colasurdo, G., Paul, S., and Said, S. I. (2002)
New evidence for transmitter role of VIP in the airways: impaired relaxation by a catalytic antibody. Pulm Pharmacol Ther 15, 121-127
59. Polonis, V. R., Brown, B. K., Rosa Borges, A., Zolla-Pazner, S., Dimitrov, D. S., Zhang, M. Y., Barnett, S. W., Ruprecht, R. M., Scarlatti, G., Fenyo, E. M., Montefiori, D.C., McCutchan, F. E., and Michael, N. L. (2008) Recent advances in the characterization of HIV-1 neutralization assays for standardized evaluation of the antibody response to infection and vaccination. Virology 375, 315-320
60. Moore, J. P., Cao, Y., Qing, L., Sattentau, Q. J., Pyati, J., Koduri, R., Robinson, J., Barbas, C. F., 3rd, Burton, D. R., and Ho, D. D. (1995) Primary isolates of human immunodeficiency virus type 1 are relatively resistant to neutralization by monoclonal antibodies to gp120, and their neutralization is not predicted by studies with monomeric gp120. J Virol 69, 101-109
61. Bures, R., Gaitan, A., Zhu, T., Graziosi, C., McGrath, K. M., Tartaglia, J., Caudrelier, P., El Habib, R., Klein, M., Lazzarin, A., Stablein, D. M., Deers, M., Corey, L., Greenberg, M. L., Schwartz, D. H., and Montefiori, D.C. (2000) Immunization with recombinant canarypox vectors expressing membrane-anchored glycoprotein 120 followed by glycoprotein 160 boosting fails to generate antibodies that neutralize R5 primary isolates of human immunodeficiency virus type 1. AIDS Res Hum Retroviruses 16, 2019-2035
62. Rusert, P., Mann, A., Huber, M., von Wyl, V., Gunthard, H. F., and Trkola, A. (2009) Divergent effects of cell environment on HIV entry inhibitor activity. Aids 23, 1319-1327
63. Brown, B. K., Wieczorek, L., Sanders-Buell, E., Rosa Borges, A., Robb, M. L., Birx, D. L., Michael, N. L., McCutchan, F. E., and Polonis, V. R. (2008) Cross-clade neutralization patterns among HIV-1 strains from the six major clades of the pandemic evaluated and compared in two different models. Virology 375, 529-538
64. Mann, A. M., Rusert, P., Berlinger, L., Kuster, H., Gunthard, H. F., and Trkola, A. (2009) HIV sensitivity to neutralization is determined by target and virus producer cell properties. Aids 23, 1659-1667
65. Yuan, W., Bazick, J., and Sodroski, J. (2006) Characterization of the multiple conformational States of free monomeric and trimeric human immunodeficiency virus envelope glycoproteins after fixation by cross-linker. J Virol 80, 6725-6737
66. el-Amad, Z., Murthy, K. K., Higgins, K., Cobb, E. K., Haigwood, N. L., Levy, J. A., and Steimer, K. S. (1995) Resistance of chimpanzees immunized with recombinant gp120SF2 to challenge by HIV-1 SF2. Aids 9, 1313-1322
67. Berman, P. W., Murthy, K. K., Wrin, T., Vennari, J. C., Cobb, E. K., Eastman, D. J., Champe, M., Nakamura, G. R., Davison, D., Powell, M. F., Bussiere, J., Francis, D. P., Matthews, T., Gregory, T. J., and Obijeski, J. F. (1996) Protection of MN-rgp120-immunized chimpanzees from heterologous infection with a primary isolate of human immunodeficiency virus type 1. J Infect Dis 173, 52-59
68. Pitisuttithum, P., Gilbert, P., Gurwith, M., Heyward, W., Martin, M., van Griensven, F., Hu, D., Tappero, J. W., and Choopanya, K. (2006) Randomized, double-blind, placebo-controlled efficacy trial of a bivalent recombinant glycoprotein 120 HIV-1 vaccine among injection drug users in Bangkok, Thailand. J Infect Dis 194, 1661-1671
69. Sekaly, R. P. (2008) The failed HIV Merck vaccine study: a step back or a launching point for future vaccine development? J Exp Med 205, 7-12
70. Shedlock, D. J., Silvestri, G., and Weiner, D. B. (2009) Monkeying around with HIV vaccines: using rhesus macaques to define ‘gatekeepers’ for clinical trials. Nat Rev Immunol 9, 717-728
71. Robey, F. A., Harris-Kelson, T., Robert-Guroff, M., Batinic, D., Ivanov, B., Lewis, M. S., and Roller, P. P. (1996) A synthetic conformational epitope from the C4 domain of HIV Gp120 that binds CD4. J Biol Chem 271, 17990-17995
72. Sun, M., Gao, Q. S., Kirnarskiy, L., Rees, A., and Paul, S. (1997) Cleavage specificity of a proteolytic antibody light chain and effects of the heavy chain variable domain. J Mol Biol 271, 374-385
73. Taguchi, H., Planque, S., Sapparapu, G., Boivin, S., Hara, M., Nishiyama, Y., and Paul, S. (2008) Exceptional amyloid beta peptide hydrolyzing activity of nonphysiological immunoglobulin variable domain scaffolds. J Biol Chem 283, 36724-36733
74. Nishiyama, Y., Mitsuda, Y., Taguchi, H., Planque, S., Hara, M., Karle, S., Hanson, C. V., Uda, T., and Paul, S. (2005) Broadly distributed nucleophilic reactivity of proteins coordinated with specific ligand binding activity. J Mol Recognit 18, 295-306
75. Forthal, D. N., and Landucci, G. (1998) In vitro reduction of virus infectivity by antibody-dependent cell-mediated immunity. J Immunol Methods 220, 129-138
76. Chang, B., and Casali, P. (1994) The CDR1 sequences of a major proportion of human germline Ig VH genes are inherently susceptible to amino acid replacement. Immunol Today 15, 367-373
77. Zolla-Pazner, S., Cohen, S., Pinter, A., Krachmarov, C., Wrin, T., Wang, S., and Lu, S. (2009) Cross-clade neutralizing antibodies against HIV-1 induced in rabbits by focusing the immune response on a neutralizing epitope. Virology 392, 82-93
78. Nishimura, Y., Igarashi, T., Haigwood, N., Sadjadpour, R., Plishka, R. J., Buckler-White, A., Shibata, R., and Martin, M. A. (2002) Determination of a statistically valid neutralization titer in plasma that confers protection against simian-human immunodeficiency virus challenge following passive transfer of high-titered neutralizing antibodies. J Virol 76, 2123-2130
79. Harouse, J. M., Gettie, A., Eshetu, T., Tan, R. C., Bohm, R., Blanchard, J., Baskin, G., and Cheng-Mayer, C. (2001) Mucosal transmission and induction of simian AIDS by CCR5-specific simian/human immunodeficiency virus SHIV(SF162P3). J Virol 75, 1990-1995
80. Hsu, M., Harouse, J. M., Gettie, A., Buckner, C., Blanchard, J., and Cheng-Mayer, C. (2003) Increased mucosal transmission but not enhanced pathogenicity of the CCR5-tropic, simian AIDS-inducing simian/human immunodeficiency virus SHIV(SF162P3) maps to envelope gp120. J Virol 77, 989-998
81. Nehete, P. N., Nehete, B. P., Manuri, P., Hill, L., Palmer, J. L., and Sastry, K. J. (2005) Protection by dendritic cells-based HIV synthetic peptide cocktail vaccine: preclinical studies in the SHIV-rhesus model. Vaccine 23, 2154-2159
82. Mitsuda, Y., Planque, S., Hara, M., Kyle, R., Taguchi, H., Nishiyama, Y., and Paul, S. (2007) Naturally occurring catalytic antibodies: evidence for preferred development of the catalytic function in IgA class antibodies. Mol Biotechnol 36, 113-122
83. Vajdy, M., Singh, M., Kazzaz, J., Soenawan, E., Ugozzoli, M., Zhou, F., Srivastava, I., Bin, Q., Barnett, S., Donnelly, J., Luciw, P., Adamson, L., Montefiori, D., and O'Hagan, D. T. (2004) Mucosal and systemic anti-HIV responses in rhesus macaques following combinations of intranasal and parenteral immunizations. AIDS Res Hum Retroviruses 20, 1269-1281
84. Bergquist, C., Johansson, E. L., Lagergard, T., Holmgren, J., and Rudin, A. (1997) Intranasal vaccination of humans with recombinant cholera toxin B subunit induces systemic and local antibody responses in the upper respiratory tract and the vagina. Infect Immun 65, 2676-2684
85. McHeyzer-Williams, L. J., Pelletier, N., Mark, L., Fazilleau, N., and McHeyzer-Williams, M. G. (2009) Follicular helper T cells as cognate regulators of B cell immunity. Curr Opin Immunol 21, 266-273
86. Granoff, D. M., Holmes, S. J., Belshe, R. B., Osterholm, M. T., McHugh, J. E., and Anderson, E. L. (1994) Effect of carrier protein priming on antibody responses to Haemophilus influenzae type b conjugate vaccines in infants. Jama 272, 1116-1121
87. Xiang, R., Primus, F. J., Ruehlmann, J. M., Niethammer, A. G., Silletti, S., Lode, H. N., Dolman, C. S., Gillies, S. D., and Reisfeld, R. A. (2001) A dual-function DNA vaccine encoding carcinoembryonic antigen and CD40 ligand trimer induces T cell-mediated protective immunity against colon cancer in carcinoembryonic antigen-transgenic mice. J Immunol 167, 4560-4565
88. Li, H., Willingham, S. B., Ting, J. P., and Re, F. (2008) Cutting edge: inflammasome activation by alum and alum's adjuvant effect are mediated by NLRP3. J Immunol 181, 17-21
89. Makidon, P. E., Bielinska, A. U., Nigavekar, S. S., Janczak, K. W., Knowlton, J., Scott, A. J., Monk, N., Cao, Z., Rathinavelu, S., Beer, M. R., Wilkinson, J. E., Blanco, L. P., Landers, J. J., and Baker, J. R., Jr. (2008) Pre-clinical evaluation of a novel nanoemulsion-based hepatitis B mucosal vaccine. PLoS One 3, e2954
90. Tengvall, S., Lundqvist, A., Eisenberg, R. J., Cohen, G. H., and Harandi, A. M. (2006) Mucosal administration of CpG oligodeoxynucleotide elicits strong CC and CXC chemokine responses in the vagina and serves as a potent Th1-tilting adjuvant for recombinant gD2 protein vaccination against genital herpes. J Virol 80, 5283-5291
91. Becker, P. D., Corral, R. S., Guzman, C. A., and Grinstein, S. (2001) Adamantylamide dipeptide as effective immunoadjuvant in rabbits and mice. Vaccine 19, 4603-4609
92. Aranha, C. C., Gupta, S. M., and Reddy, K. V. (2008) Assessment of cervicovaginal cytokine levels following exposure to microbicide Nisin gel in rabbits. Cytokine 43, 63-70
93. Inoue, H., Fukuizumi, T., Tsujisawa, T., and Uchiyama, C. (1999) Simultaneous induction of specific immunoglobulin A—producing cells in major and minor salivary glands after tonsillar application of antigen in rabbits. Oral Microbiol Immunol 14, 21-26
94. Kaiser, E. T., and Kezdy, F. J. (1987) Peptides with affinity for membranes. Annu Rev Biophys Biophys Chem 16, 561-581
95. Schafmeister, C. E., Po, J., and Verdine, G. L. (2000) An All-Hydrocarbon Cross-Linking System for Enhancing the Helicity and Metabolic Stability of Peptides. J. Am. Chem. Soc. 122, 5891-5892
96. Siedlecka, M., Goch, G., Ejchart, A., Sticht, H., and Bierzynski, A. (1999) Alphα-helix nucleation by a calcium-binding peptide loop. Proc Natl Acad Sci USA 96, 903-908
97. Gololobov, G., Noda, Y., Sherman, S., Rubinstein, I., Baranowska-Kortylewicz, J., and Paul, S. (1998) Stabilization of vasoactive intestinal peptide by lipids. J Pharmacol Exp Ther 285, 753-758
98. Marks, J. D., Hoogenboom, H. R., Bonnert, T. P., McCafferty, J., Griffiths, A. D., and Winter, G. (1991) By-passing immunization. Human antibodies from V-gene libraries displayed on phage. J Mol Biol 222, 581-597
99. Lipovsek, D., and Pluckthun, A. (2004) In-vitro protein evolution by ribosome display and mRNA display. J Immunol Methods 290, 51-67
100. Li, Q., Anver, M. R., Li, Z., Butcher, D. O., and Gildersleeve, J. C. (2009) GalNAcalpha 1-3Gal, a new prognostic marker for cervical cancer. Int J Cancer 126, 459-468
101. Pope, M. E., Soste, M. V., Eyford, B. A., Anderson, N. L., and Pearson, T. W. (2009) Anti-peptide antibody screening: selection of high affinity monoclonal reagents by a refined surface plasmon resonance technique. J Immunol Methods 341, 86-96
102. Marx, P. A., Spira, A. I., Gettie, A., Dailey, P. J., Veazey, R. S., Lackner, A. A., Mahoney, C. J., Miller, C. J., Claypool, L. E., Ho, D. D., and Alexander, N.J. (1996) Progesterone implants enhance SIV vaginal transmission and early virus load. Nat Med 2, 1084-1089
103. Romano, J. W., Shurtliff, R. N., Sarngadharan, M. G., and Pal, R. (1995) Detection of HIV-1 infection in vitro using NASBA: an isothermal RNA amplification technique. J Virol Methods 54, 109-119
104. Lee, E. M., Chung, H. K., Livesay, J., Suschak, J., Finke, L., Hudacik, L., Galmin, L., Bowen, B., Markham, P., Cristillo, A., and Pal, R. (2009) Molecular methods for evaluation of virological status of nonhuman primates challenged with simian immunodeficiency or simian-human immunodeficiency viruses. J Virol Methods
105. Cristillo, A. D., Lisziewicz, J., He, L., Lori, F., Galmin, L., Trocio, J. N., Unangst, T., Whitman, L., Hudacik, L., Bakare, N., Whitney, S., Restrepo, S., Suschak, J., Ferrari, M. G., Chung, H. K., Kalyanaraman, V. S., Markham, P., and Pal, R. (2007) HIV-1 prophylactic vaccine comprised of topical DermaVir prime and protein boost elicits cellular immune responses and controls pathogenic R5SHIV162P3. Virology 366, 197-211
106. Sundling, C., Schon, K., Morner, A., Forsell, M. N., Wyatt, R. T., Thorstensson, R., Karlsson Hedestam, G. B., and Lycke, N.Y. (2008) CTA™-DD adjuvant promotes strong immunity against human immunodeficiency virus type 1 envelope glycoproteins following mucosal immunization. J Gen Virol 89, 2954-2964
107. Ioannou, X. P., Gomis, S. M., Hecker, R., Babiuk, L. A., and van Drunen Littel-van den Hurk, S. (2003) Safety and efficacy of CpG-containing oligodeoxynucleotides as immunological adjuvants in rabbits. Vaccine 21, 4368-4372
108. Lowell, G. H., Kaminski, R. W., Grate, S., Hunt, R. E., Charney, C., Zimmer, S., and Colleton, C. (1996) Intranasal and intramuscular proteosome-staphylococcal enterotoxin B (SEB) toxoid vaccines: immunogenicity and efficacy against lethal SEB intoxication in mice. Infect Immun 64, 1706-1713
109. Lin, L., Hanson, C. V., Alter, H. J., Jauvin, V., Bernard, K. A., Murthy, K. K., Metzel, P., and Corash, L. (2005) Inactivation of viruses in platelet concentrates by photochemical treatment with amotosalen and long-wavelength ultraviolet light. Transfusion 45, 580-590
110. Mody, R., Tramontano, A., and Paul, S. (1994) Spontaneous hydrolysis of vasoactive intestinal peptide in neutral aqueous solution. Int J Pept Protein Res 44, 441-447
111. Ruppach, H., Nara, P., Raudonat, I., Elanjikal, Z., Rubsamen-Waigmann, H., and Dietrich, U. (2000) Human immunodeficiency virus (HIV)-positive sera obtained shortly after seroconversion neutralize autologous HIV type 1 isolates on primary macrophages but not on lymphocytes. J Virol 74, 5403-5411
112. Mo, H., Stamatatos, L., Ip, J. E., Barbas, C. F., Parren, P. W., Burton, D. R., Moore, J. P., and Ho, D. D. (1997) Human immunodeficiency virus type 1 mutants that escape neutralization by human monoclonal antibody IgG1b12. off. J Virol 71, 6869-6874
113. Kirchherr, J. L., Lu, X., Kasongo, W., Chalwe, V., Mwananyanda, L., Musonda, R. M., Xia, S. M., Scearce, R. M., Liao, H. X., Montefiori, D.C., Haynes, B. F., and Gao, F. (2007) High throughput functional analysis of HIV-1 env genes without cloning. J Virol Methods 143, 104-111

Each of the patent applications, patents and other publications cited herein is incorporated by reference in its entirety.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

1. A synthetic immunogen with a conformation similar to the conformation of the CD4 binding site of gp120 on the surface of HIV that induces neutralizing antibodies to genetically diverse Group M HIV-1 strains, having the formula
L-E
wherein L is a peptide sequence that comprises fourteen or more amino acids selected from the region of gp120 containing the consensus amino acids 406-459 numbered according to the HXB2 numbering system, or a mimotope thereof, and E is an electrophilic group covalently linked to an amino acid side chain of L, having the formula
Y-Y′-Y″
wherein
Y is an electrophilic group,
Y′ is a charged or neutral group, or is absent, and
Y″ is a linker, covalent bond or atom.

2. The synthetic immunogen of claim 1, where the immunogen is effective to induce the synthesis of HIV neutralizing antibodies to the 421-433 CD4 binding site sequence by recognition of the framework regions of B cell receptors.

3. The synthetic immunogen of claim 1 wherein L comprises peptide sequence 416-433.

4. The synthetic immunogen of claim 1 wherein L comprises peptide sequence 414-439.

5. The synthetic immunogen of claim 1, having the formula:
Cys-Leu-Pro-Ser-Arg-Ile-Lys(X)-Gln-Ile-Ile-Asn-Met-Trp-Gln-Glu-Val-Gly-Lys(X)-Ala (SEQ ID NO:1), or
Ile-Thr-Cys-Leu-Pro-Ser-Arg-Ile-Lys(X)-Gln-Ile-Ile-Asn-Met-Trp-Gln-Glu-Val-Gly-Lys(X)-Ala-Met-Tyr-Ala-Pro-Pro-Ile (SEQ ID NO:2),
wherein X is an electrophilic group of formula: 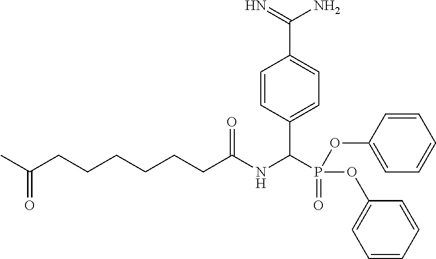
and Lys(X) indicates X is covalently linked to a side chain of the lysine residue.

6. An immunogenic composition, comprising:
a synthetic immunogen according to claim 1; and
an adjuvant.

7. An immunogenic compound comprising the synthetic immunogen of claim 1 conjugated to a carrier molecule.

8. The immunogenic compound of claim 7, wherein the carrier molecule promotes folding of the synthetic immunogen into a conformation similar to the conformation of the CD4 binding site of gp120 on the surface of HIV.

9. The immunogenic compound of claim 7, wherein the carrier molecule is selected from the group consisting of keyhole limpet hemocyanin, tetanus toxoid, and CD40 ligand.

10. An immunogenic composition comprising:
the immunogenic compound of claim 7; and
an adjuvant.

11. A method for producing HIV neutralizing antibodies to genetically diverse Group M HIV-1 strains within an organism capable of producing antibodies comprising:
administering a synthetic immunogen according to claim 1 to the organism in an amount effective to cause production of neutralizing antibodies against the CD4-binding site of HIV gp120.

12. The method of claim 11, wherein the administering step further comprises administering an adjuvant to the mammal.

13. A method for producing HIV neutralizing antibodies to genetically diverse Group M HIV-1 strains within an organism capable of producing antibodies comprising:
administering an immunogen according to claim 7 to the organism in an amount effective to cause production of neutralizing antibodies against the CD4-binding site of HIV gp120.

14. The method of claim 13, wherein the administering step further comprises administering an adjuvant to the organism.

15. The synthetic immunogen of claim 1, wherein L is a peptide sequence that comprises fourteen or more amino acids selected from the region of gp120 containing amino acids 406-459 or a mimotope thereof and a second epitope of gp120 or a mimotope thereof.

16. The synthetic immunogen of claim 1, further comprising one or more cross-links between amino acids that rigidify the conformation.

17. A synthetic immunogen with a conformation similar to the conformation of the CD4 binding site of gp120 on the surface of HIV that induces neutralizing antibodies to genetically diverse Group M HIV-1 strains, having the formula
L-E
wherein L is a peptide sequence that comprises fourteen or more amino acids selected from the region of gp120 containing amino acids 406-459 with one or more amino acid sequence differences compared to the consensus sequence of amino acids 406-459 of Group M HIV-1 gp120, and E is an electrophilic group covalently linked to an amino acid side chain of L, having the formula
Y-Y′-Y″
wherein
Y is an electrophilic group,
Y′ is a charged or neutral group, or is absent, and
Y″ is a linker, covalent bond or atom.

18. A method for the preparation of an immunogen with a conformation similar to the CD4 binding site of gp120 on the surface of HIV that induces neutralizing antibodies to genetically diverse Group M HIV-1 strains, said immunogen having the formula
L-E
wherein L is gp120 and E is an electrophilic group conjugated to a side chain functional group of L having the formula
Y-Y′-Y″
wherein
Y is an electrophilic group,
Y′ is a charged or neutral group, or is absent, and
Y″ is a linker, covalent bond or atom,
comprising the steps of:
(a) conjugating varying numbers of Y-Y′Y″ groups per molecule of gp120;
(b) incubating the resultant L-E preparation for a length of time sufficient to enable intermolecular covalent bonding between the L-E molecules
(c) fractionating the L-E preparation into multiple fractions containing individual subpopulations of L-E molecules characterized by their size, charge, hydrophobicity or conformation;
(d) assaying the several variant L-E fractions from step (c) to determine their CD4 binding activity or their antibody binding activity; and
(e) identifying those variant L-E fractions from step (c) that induce the greatest synthesis of HIV neutralizing antibodies in an organism.

19. A method for the preparation of an immunogen that induces neutralizing antibodies to genetically diverse Group M HIV-1 strains, said synthetic immunogen composed of intact HIV-1 particles having on their surface molecules with the formula
L-E
wherein L is gp120 and E is an electrophilic group conjugated to a side chain functional group of L having the formula
Y-Y′-Y″
wherein
Y is an electrophilic group,
Y′ is a charged or neutral group, or is absent, and
Y″ is a linker, covalent bond or atom,
comprising the steps of:
(a) conjugating varying numbers of Y-Y′Y″ groups per gp120 molecule expressed on the surface of intact HIV-1;
(b) incubating the resultant HIV-1 particles for a length of time sufficient to enable intermolecular covalent bonding between the surface L-E molecules
(c) fractionating the HIV-1 particles with surface L-E molecules into multiple fractions containing individual subpopulations of HIV-1 particles characterized by their size, charge, hydrophobicity or conformation;
(d) assaying the several variant HIV-1 fractions from step (c) to determine their CD4 binding activity or their antibody binding activity; and
(e) identifying those variant HIV-1 fractions from step (c) to induce the greatest synthesis of HIV neutralizing antibodies in an organism.

20. An isolated polypeptide, comprising the framework regions (non-underlined) of at least one of the following antibody V_{L} and V_{H} amino acid sequences:
IgM clone G12
(a) VL chain:
(SEQ ID NO: 3)
ENVLTQSPAIMSASPGEKVTMTCSASSSVSYMHWYQQKSSTSPKLWIYDT
SKLASGVPGRFSGSGSGNSYSLTISSMEAEDVATYYCFQGSGYPYTFGGG
TKLEIK;
(b) VH chain:
(SEQ ID NO: 4)
QVQLQQPGSVLVRPGASVKLSCKASGYTFTSSWMHWAKQRPGQGLEWIGE
IHPNSGNTNYNEKFKGKATLTVGTSSSTAYVDLSSLTSEDSAVYYCARPG
IGESQSFPNVFPAAAEXLKGEFCRYPSHWRPLEHAS;
IgM clone C11:
(c) VL chain:
(SEQ ID NO: 5)
DIQMTQSPATLSVTPGDSVSLSCRASQSISNNLHWYQQKSHESPRLLIKY
ASQSISGIPSRFSGSGSGTDFTLSINSVETEDFGMYFCQQSNSWPLTFGA
GTKLELK;
(d) VH chain:
(SEQ ID NO: 6)
VQVQLKQSGPGLVQPSQSISITCTVSGFSLTSYGVHWVRQSPGKGLEWLG
VIWSGGSTDYNAAFISRLSISKDNSKSQVFFKMNSLQANDTAIYYCARTG
FAYWGRGTLVTVS;
IgM clone H10:
(e) VL chain:
(SEQ ID NO: 7)
QIVLTQSPAIMSASLGERVTMTCTASSSVSSSYLHWYQQKPGSSPKLWIY
STSNLASGVPARFSGSGSGTSYSLTISSMEAEDAATYYCHQYHRSPRTFG
GGTKLEIK;
(f) VH chain:
(SEQ ID NO: 8)
EVKLVESGGGLVQPGGSLRLSCATSGFTFTDYYMSWVRQPPGKALERLGF
IRNKANGYTTEYSASVKGRFTISRDNSQSILYLQMNTLRAEDSATYYCAR
DNQSFYYAMDYWGQGTSVTVSS;
IgM clone 1F4:
(g) VL chain:
(SEQ ID NO: 9)
VLMTQTPLSLPVSLGDQASISCRSSQSLVHSNGNTYLHWYLQKPGQSPKL
LIYKVSNRFSGVPDRFSGSGSGTDFTLKISRVEAEDLGVYFCSQSTHVPY
TFGGGTKLEIK;
(h) VH chain:
(SEQ ID NO: 10)
EVKLQESGPSLVQPSQSLSITCTVSGFSLTSYGVHWVRQSPGKGLEWLGV
IWRGGSTDYNAAFMSRLSITKDNSKSQVFFKMNSLQADDTAIYYCAKRYG
NYGGGAMDYWGQGTSVTVSS;
IgM clone 2C11:
(i) VL chain:
(SEQ ID NO: 11)
QIVLTQSPAIMSASPGEKVTITCSASSSVSYMHWFQQKPGTSPKLWIYST
SNLASGVPARFSGSGSGTSYSLTISRMEAEDAATYYCQQRSSYPYTFGGG
TKLEIK;
(j) VH chain:
(SEQ ID NO: 12)
EVQLVESGGGLVQPKGSLKLSCAASGFTFNTYAMHWVCQAPGKGLECVAR
IRSKSNNYATYYADSVKDRFTISRDDSQSMLYLQMNNLKTEDTAMYYCVR
ERAGYFDVWGAGTTVTVSS;
IgM Clone 2G2:
(k) VL chain:
(SEQ ID NO: 13)
DIVITQSPSYLAASPGETITINCRASKSISKYLAWYQEKPGKTNKLLIYS
GSTLQSGIPSRFSGSGSGTDFTLTISSLEPEDFAMYYCQQHNEYPYTFGG
GTKLEIK;
(l) VH chain:
(SEQ ID NO: 14)
EVQLQQSGPELVKTGASVKISCKASGYSFTGYYMHWVKQSHGKSLEWIGY
ISCYNGATSYNQKFKGKATFTVDTSSSTAYMQFNSLTSEDSAVYYCARGG
TTVVATGKYAMDYWGQGTSVTVSS;
IgM clone 2G9:
(m) VL chain:
(SEQ ID NO: 15)
DIQMTQTTSSLSASLGDRVTISCRASQDISNYLNWYQQKPDGTVKLLIYY
TSRLHSGVPSRFSGSGSGTDYSLTISNLEQEDIATYFCQQGNTLPTFGGG
TKLEIKRA;
(n) VH chain:
(SEQ ID NO: 16)
QVQLKQSGPGLVQPSQSLSITCTVSGFSLTSYGVHWVRQSPGKGLEWLGV
IWSGGSTDYNAAFISRLSISKDNSKSQVFFKMNSLQANDTAIYYCARNKD
YGSSYDYYAMDYWGQGTSVTVSS;
IgG clone 9F3:
(o) VL chain:
(SEQ ID NO: 17)
DIVMSQSPSSLAVSAGEKVTMRCKSSQSLLNSRTRKNYLAWYQQKPGQSP
KLLIYWASTRESGVPDRFTGSGSGTDFTLTISSVQAEDLAVYYCKQFYNL
WTFGGGTKLEIK;
(p) VH chain:
(SEQ ID NO: 18)
QVQLQQSGAELVRPGASVKLSCKALGYTFTDYEMHWVKQTPVHGLEWIGG
IYPGSGGTAYNQKFKGKATLTADKSSSTAYMELSSLTSEDSAVYYCTKFR
FSSFAMDYWGQGTSVTVSS;
IgG clone 4B2:
(q) VL chain
(SEQ ID NO: 19)
DIVMSQSPSSLAVSAGEKVTMSCKSSQSLLNSRTRKNYLAWYQQKPGQSP
KLLIYWASTRESGVPDRFTGSGSGTDFTLTINSVQAEDLAVYYCKQSYNL
WTFGGGTKLEIK;
and
(r) VH chain:
(SEQ ID NO: 20)
QVQLQQSGAELVRPGASVKLSCMALGYTFTDYEIHWVKQTPVHGLEWIGG
FHPGSGGGAYSQKFKGKATLIADKSSSIAYMEVISLTSEDSAVYYCTRFR
YSSFAMVYWGQGTSVTVSS,
wherein underlined sequences are CDRs and non-underlined sequences are antibody framework regions.

21. The isolated polypeptide of claim 20, comprising at least one of the antibody V_{L} and V_{H} amino acid sequences.

22. The isolated polypeptide of claim 20, wherein the polypeptide is an antibody.


SEQUENCE LISTING (45 sequences)

SEQ ID NO: 1 - protein, 19 aa, Artificial Sequence
  Description of Artificial Sequence Synthetic peptide
CLPSRIKQIINMWQEVGKA

SEQ ID NO: 2 - protein, 27 aa, Artificial Sequence
  Description of Artificial Sequence Synthetic peptide
ITCLPSRIKQIINMWQEVGKAMYAPPI

SEQ ID NO: 3 - protein, 106 aa, Artificial Sequence
  Description of Artificial Sequence Synthetic polypeptide
ENVLTQSPAIMSASPGEKVTMTCSASSSVSYMHWYQQKSSTSPKLWIYDTSKLASGVPGRFSGSGSGNSYSLTISSMEAEDVATYYCFQGSGYPYTFGGGTKLEIK

SEQ ID NO: 4 - protein, 136 aa, Artificial Sequence
  Description of Artificial Sequence Synthetic polypeptide
QVQLQQPGSVLVRPGASVKLSCKASGYTFTSSWMHWAKQRPGQGLEWIGEIHPNSGNTNYNEKFKGKATLTVGTSSSTAYVDLSSLTSEDSAVYYCARPGIGESQSFPNVFPAAAEXLKGEFCRYPSHWRPLEHAS

SEQ ID NO: 5 - protein, 107 aa, Artificial Sequence
  Description of Artificial Sequence Synthetic polypeptide
DIQMTQSPATLSVTPGDSVSLSCRASQSISNNLHWYQQKSHESPRLLIKYASQSISGIPSRFSGSGSGTDFTLSINSVETEDFGMYFCQQSNSWPLTFGAGTKLELK

SEQ ID NO: 6 - protein, 113 aa, Artificial Sequence
  Description of Artificial Sequence Synthetic polypeptide
VQVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCARTGFAYWGRGTLVTVS

SEQ ID NO: 7 - protein, 108 aa, Artificial Sequence
  Description of Artificial Sequence Synthetic polypeptide
QIVLTQSPAIMSASLGERVTMTCTASSSVSSSYLHWYQQKPGSSPKLWIYSTSNLASGVPARFSGSGSGTSYSLTISSMEAEDAATYYCHQYHRSPRTFGGGTKLEIK

SEQ ID NO: 8 - protein, 122 aa, Artificial Sequence
  Description of Artificial Sequence Synthetic polypeptide
EVKLVESGGGLVQPGGSLRLSCATSGFTFTDYYMSWVRQPPGKALERLGFIRNKANGYTTEYSASVKGRFTISRDNSQSILYLQMNTLRAEDSATYYCARDNQSFYYAMDYWGQGTSVTVSS

SEQ ID NO: 9 - protein, 111 aa, Artificial Sequence
  Description of Artificial Sequence Synthetic polypeptide
VLMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIK

SEQ ID NO: 10 - protein, 120 aa, Artificial Sequence
  Description of Artificial Sequence Synthetic polypeptide
EVKLQESGPSLVQPSQSLSITCTVSGFSLTSYGVHWVRQSPGKGLEWLGVIWRGGSTDYNAAFMSRLSITKDNSKSQVFFKMNSLQADDTAIYYCAKRYGNYGGGAMDYWGQGTSVTVSS

SEQ ID NO: 11 - protein, 106 aa, Artificial Sequence
  Description of Artificial Sequence Synthetic polypeptide
QIVLTQSPAIMSASPGEKVTITCSASSSVSYMHWFQQKPGTSPKLWIYSTSNLASGVPARFSGSGSGTSYSLTISRMEAEDAATYYCQQRSSYPYTFGGGTKLEIK

SEQ ID NO: 12 - protein, 119 aa, Artificial Sequence
  Description of Artificial Sequence Synthetic polypeptide
EVQLVESGGGLVQPKGSLKLSCAASGFTFNTYAMHWVCQAPGKGLECVARIRSKSNNYATYYADSVKDRFTISRDDSQSMLYLQMNNLKTEDTAMYYCVRERAGYFDVWGAGTTVTVSS

SEQ ID NO: 13 - protein, 107 aa, Artificial Sequence
  Description of Artificial Sequence Synthetic polypeptide
DIVITQSPSYLAASPGETITINCRASKSISKYLAWYQEKPGKTNKLLIYSGSTLQSGIPSRFSGSGSGTDFTLTISSLEPEDFAMYYCQQHNEYPYTFGGGTKLEIK

SEQ ID NO: 14 - protein, 124 aa, Artificial Sequence
  Description of Artificial Sequence Synthetic polypeptide
EVQLQQSGPELVKTGASVKISCKASGYSFTGYYMHWVKQSHGKSLEWIGYISCYNGATSYNQKFKGKATFTVDTSSSTAYMQFNSLTSEDSAVYYCARGGTTVVATGKYAMDYWGQGTSVTVSS

SEQ ID NO: 15 - protein, 108 aa, Artificial Sequence
  Description of Artificial Sequence Synthetic polypeptide
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPTFGGGTKLEIKRA

SEQ ID NO: 16 - protein, 123 aa, Artificial Sequence
  Description of Artificial Sequence Synthetic polypeptide
QVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCARNKDYGSSYDYYAMDYWGQGTSVTVSS

SEQ ID NO: 17 - protein, 112 aa, Artificial Sequence
  Description of Artificial Sequence Synthetic polypeptide
DIVMSQSPSSLAVSAGEKVTMRCKSSQSLLNSRTRKNYLAWYQQKPGQSPKLLIYWASTRESGVPDRFTGSGSGTDFTLTISSVQAEDLAVYYCKQFYNLWTFGGGTKLEIK

SEQ ID NO: 18 - protein, 119 aa, Artificial Sequence
  Description of Artificial Sequence Synthetic polypeptide
QVQLQQSGAELVRPGASVKLSCKALGYTFTDYEMHWVKQTPVHGLEWIGGIYPGSGGTAYNQKFKGKATLTADKSSSTAYMELSSLTSEDSAVYYCTKFRFSSFAMDYWGQGTSVTVSS

SEQ ID NO: 19 - protein, 112 aa, Artificial Sequence
  Description of Artificial Sequence Synthetic polypeptide
DIVMSQSPSSLAVSAGEKVTMSCKSSQSLLNSRTRKNYLAWYQQKPGQSPKLLIYWASTRESGVPDRFTGSGSGTDFTLTINSVQAEDLAVYYCKQSYNLWTFGGGTKLEIK

SEQ ID NO: 20 - protein, 119 aa, Artificial Sequence
  Description of Artificial Sequence Synthetic polypeptide
QVQLQQSGAELVRPGASVKLSCMALGYTFTDYEIHWVKQTPVHGLEWIGGFHPGSGGGAYSQKFKGKATLIADKSSSIAYMEVISLTSEDSAVYYCTRFRYSSFAMVYWGQGTSVTVSS

SEQ ID NO: 21 - protein, 18 aa, Artificial Sequence
  Description of Artificial Sequence Synthetic peptide
GQKSWEIPAKNRLIMVIQ

SEQ ID NO: 22 - protein, 49 aa, Human immunodeficiency virus I
NNTITLPCRIKQIINMWQGVGQAMYAPPIEGKIRCTSNITGLLLTRDGG

SEQ ID NO: 23 - DNA, 22 bp, Artificial Sequence
  Description of Artificial Sequence Synthetic oligonucleotide
tcgtcgttgtcgttttgtcgtt

SEQ ID NO: 24 - protein, 18 aa, Artificial Sequence
  Description of Artificial Sequence Synthetic consensus
XPCXIXQXXXXWQXXGXA

SEQ ID NO: 25 - protein, 18 aa, Artificial Sequence
  Description of Artificial Sequence Synthetic consensus
XPXXIXQXXXXWQXXGXA

SEQ ID NO: 26 - protein, 18 aa, Artificial Sequence
  Description of Artificial Sequence Synthetic consensus
LXCRIKQIIXXWQXVGKA

SEQ ID NO: 27 - protein, 18 aa, Artificial Sequence
  Description of Artificial Sequence Synthetic consensus
LXXRIKQIIXXWQXVGKA

SEQ ID NO: 28 - protein, 11 aa, Artificial Sequence
  Description of Artificial Sequence Synthetic peptide
KQIINMWQEVG

SEQ ID NO: 29 - protein, 12 aa, Artificial Sequence
  Description of Artificial Sequence Synthetic peptide
CKQIINMWQEVG

SEQ ID NO: 30 - protein, 14 aa, Artificial Sequence
  Description of Artificial Sequence Synthetic peptide
CKQIINMWQEVGKA

SEQ ID NO: 31 - protein, 27 aa, Artificial Sequence
  Description of Artificial Sequence Synthetic peptide
CITLPCRIKQIINMWQEVGKAMYAPPI

SEQ ID NO: 32 - protein, 29 aa, Artificial Sequence
  Description of Artificial Sequence Synthetic peptide
CHSDAVFTDNYTRLRKQMAVKKYLNSILN

SEQ ID NO: 33 - protein, 19 aa, Artificial Sequence
  Description of Artificial Sequence Synthetic peptide
CGQKSWEIPAKNRLIMVIQ

SEQ ID NO: 34 - protein, 18 aa, Artificial Sequence
  Description of Artificial Sequence Synthetic peptide
LPSRIKQIINMWQEVGKA

SEQ ID NO: 35 - protein, 18 aa, Artificial Sequence
  Description of Artificial Sequence Synthetic peptide
LPCRIKQIINMWQEVGKA

SEQ ID NO: 36 - protein, 18 aa, Artificial Sequence
  Description of Artificial Sequence Synthetic peptide
LPCRIKQIINMWQEVGRA

SEQ ID NO: 37 - protein, 23 aa, Artificial Sequence
  Description of Artificial Sequence Synthetic peptide
NNTRKSIHIGPGRAFYTTGEIIG

SEQ ID NO: 38 - protein, 23 aa, Artificial Sequence
  Description of Artificial Sequence Synthetic peptide
NNTRRSVHIGPGSAFYTTGEIIG

SEQ ID NO: 39 - protein, 23 aa, Artificial Sequence
  Description of Artificial Sequence Synthetic peptide
NNTRKSMRIGPGQVFYATNGIIG

SEQ ID NO: 40 - protein, 18 aa, Artificial Sequence
  Description of Artificial Sequence Synthetic peptide
LPXRIKXIIXMWQEVGKA

SEQ ID NO: 41 - protein, 18 aa, Artificial Sequence
  Description of Artificial Sequence Synthetic peptide
LPCRIKQIINRWQEVGKA

SEQ ID NO: 42 - protein, 18 aa, Artificial Sequence
  Description of Artificial Sequence Synthetic peptide
LPCRIKQIVNMWQRVGKA

SEQ ID NO: 43 - protein, 18 aa, Artificial Sequence
  Description of Artificial Sequence Synthetic peptide
LQCRIKQIINMWQEVGRA

SEQ ID NO: 44 - protein, 18 aa, Artificial Sequence
  Description of Artificial Sequence Synthetic peptide
LQCRIKQIINMWQEVGKA

SEQ ID NO: 45 - protein, 18 aa, Artificial Sequence
  Description of Artificial Sequence Synthetic peptide
IPCRIKQIINMWQGVGKA